(19) United States
(12) Patent Application Publication
(10) Pub. No.: US 2014/0171434 A1
(43) Pub. Date: Jun. 19, 2014

(54) SUBSTITUTED IMIDAZOPYRIDINES AND IMIDAZOPYRIDAZINES AND THE USE THEREOF

(71) Applicants: Markus Follmann, Koln (DE); Johannes-Peter Stasch, Solingen (DE); Gorden Redlich, Bochum (DE); Nils Griebenow, Dormagen (DE); Dieter Lang, Velbert (DE); Frank Wunder, Wuppertal (DE); Volkhart Min-Jian Li, Velbert (DE)

(72) Inventors: Markus Follmann, Koln (DE); Johannes-Peter Stasch, Solingen (DE); Gorden Redlich, Bochum (DE); Nils Griebenow, Dormagen (DE); Dieter Lang, Velbert (DE); Frank Wunder, Wuppertal (DE); Volkhart Min-Jian Li, Velbert (DE)

(73) Assignees: BAYER PHARMA AKTIENGESSELLSCHAFT, Berlin (DE); BAYER INTELLECTUAL PROPERTY GMBH, Monheim (DE)

(21) Appl. No.: 14/115,870

(22) PCT Filed: May 2, 2012
(86) PCT No.: PCT/EP2012/058046

§ 371 Date: Feb. 5, 2014

(30) Foreign Application Priority Data
Jan. 11, 2011 (DE) 102012200356.8
May 6, 2011 (DE) 102011075399.0

(51) Int. Cl. C07D 471/04; A61K 31/519; A61K 45/06; C07D 487/04

(52) U.S. Cl. CPC C07D 471/04; C07D 487/04; A61K 31/519; A61K 45/06
USPC 514/243; 544/236; 514/248; 544/280; 514/265.1; 544/184

(57) ABSTRACT

The present application relates to novel substituted imidazopyridines and imidazopyridazines, to processes for their preparation, to their use, alone or in combinations, for the treatment and/or prophylaxis of diseases and to their use for the production of medicaments for the treatment and/or prophylaxis of diseases, in particular for the treatment and/or prophylaxis of cardiovascular disorders.

The present application relates to novel substituted imidazopyridines and imidazopyridazines, to processes for their preparation, to their use, alone or in combinations, for the treatment and/or prophylaxis of diseases and to their use for the production of medicaments for the treatment and/or prophylaxis of diseases, in particular for the treatment and/or prophylaxis of cardiovascular disorders.

One of the most important cellular transmission systems in mammalian cells is cyclic guanosine monophosphate (cGMP). Together with nitrogen monoxide (NO), which is released from the endothelium and transmits hormonal and mechanical signals, it forms the NO/cGMP system. Guanylate cyclases catalyse the biosynthesis of cGMP from guanosine triphosphate (GTP). The representatives of this family known to date can be divided into two groups either according to structural features or according to the type of ligands: the particulate guanylate cyclases which can be stimulated by natriuretic peptides, and the soluble guanylate cyclases which can be stimulated by NO. The soluble guanylate cyclases consist of two subunits and very probably contain one heme per heterodimer, which is part of the regulatory site. This is of central importance for the activation mechanism. NO can bind to the iron atom of heme and thus markedly increase the activity of the enzyme. Heme-free preparations cannot, by contrast, be stimulated by NO. Carbon monoxide (CO) is also able to bind to the central iron atom of heme, but the stimulation by CO is much less than that by NO.

By forming cGMP, and owing to the resulting regulation of phosphodiesterases, ion channels and protein kinases, guanylate cyclase plays an important role in various physiological processes, in particular in the relaxation and proliferation of smooth muscle cells, in platelet aggregation and platelet adhesion and in neuronal signal transmission, and also in disorders which are based on a disruption of the abovementioned processes. Under pathophysiological conditions, the NO/cGMP system can be suppressed, which can lead, for example, to hypertension, platelet activation, increased cell proliferation, endothelial dysfunction, atherosclerosis, angina pectoris, heart failure, myocardial infarction, thromboses, stroke and sexual dysfunction.

Owing to the expected high efficiency and low level of side effects, a possible NO-independent treatment for such disorders by targeting the influence of the cGMP signal pathway in organisms is a promising approach.

Therapeutic stimulation of soluble guanylate cyclase has to date been accomplished using exclusively compounds such as organic nitrates, the effect of which is based on NO. The latter is formed by bioconversion and activates soluble guanylate cyclase by attack at the central iron atom of heme. In addition to the side effects, the development of tolerance is one of the crucial disadvantages of this mode of treatment.

In recent years, some substances have been described which stimulate soluble guanylate cyclase directly, i.e. without prior release of NO, such as, for example 3-(5′-hydroxymethyl-2′-furyl)-1-benzylindazole [YC-1; Wu et al., Blood 84 (1994), 4226; Mülsch et al., Brit. J. Pharmacol. 120 (1997), 681], fatty acids [Goldberg et al., J. Biol. Chem. 252 (1977), 1279], diphenyliodonium hexafluorophosphate [Pettibone et al., Eur. J. Pharmacol. 116 (1985), 307], isoliquiritigenin [Yu et al., Brit. J. Pharmacol. 114 (1995), 1587] and various substituted pyrazole derivatives (WO 98/16223).

As stimulators of soluble guanylate cyclase, WO 00/06569 discloses fused pyrazole derivatives, and WO 03/095451 carbamate-substituted 3-pyrimidinylpyrazolopyridines. WO 2008/031513 describes inter alia substituted imidazopyridines and imidazopyrimidines as stimulators of soluble guanylate cyclase. 4-Amino-5,5-dimethyl-5,7,dihydro-6H-pyrrolo[2,3-d]pyrimidones having imidazopyridine and -pyrimidine substituents are disclosed as sGC activators in WO 2010/065275 and WO 2011/149921.

It was an object of the present invention to provide novel substances which act as stimulators of soluble guanylate cyclase and which have an identical or improved therapeutic profile compared to compounds known from the prior art, for example with respect to their in vivo properties such as their pharmacokinetic and pharmacodynamic behavior and/or their metabolic profile and/or their dose-activity relationship.

The present invention provides compounds of the general formula (I)

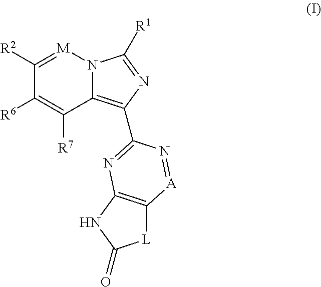

in which
A is nitrogen or CR³,
   where
   R³ is hydrogen, deuterium, halogen, difluoromethyl, trifluoromethyl, (C₁-C₄)-alkyl, (C₂-C₄)-alkenyl, (C₂-C₄)-alkynyl, cyclopropyl, cyclobutyl, hydroxy, phenyl or 5- or 6-membered heteroaryl,
   in which (C₁-C₄)-alkyl, (C₂-C₄)-alkenyl, (C₂-C₄)-alkynyl, phenyl and 5- or 6-membered heteroaryl may be substituted by 1 to 3 substituents independently of one another selected from the group consisting of fluorine, difluoromethyl, trifluoromethyl, (C₁-C₄)-alkyl, difluoromethoxy, trifluoromethoxy, (C₁-C₄)-alkoxy, (C₁-C₄)-alkoxycarbonyl, cyclopropyl and cyclobutyl,
L is a #-CR^{4A}R^{4B}—(CR^{5A}R^{5B})ₚ-## group
   where
   # is the attachment site to the carbonyl group,
   ## is the point of attachment to the pyrimidine or triazine ring,
   p is a number 0, 1 or 2,
   R^{4A} is hydrogen, fluorine, (C₁-C₄)-alkyl, hydroxy or amino,
      in which (C₁-C₄)-alkyl may be substituted by 1 to 3 substituents independently of one another selected from the group consisting of fluorine, trifluoromethyl, hydroxy, hydroxycarbonyl, (C₁-C₄)-alkoxycarbonyl and amino,
   R^{4B} is hydrogen, fluorine, difluoromethyl, trifluoromethyl, (C₁-C₆)-alkyl, (C₁-C₄)-alkoxycarbonylamino, cyano, (C₃-C₇)-cycloalkyl, difluoromethoxy, trifluoromethoxy, phenyl or a group of the formula -Q-R⁸,
      in which (C₁-C₆)-alkyl may be substituted by 1 to 3 substituents independently of one another selected from the group consisting of fluorine, cyano, trifluoromethyl, (C₃-C₇)-cycloalkyl, hydroxy, difluoromethoxy, trifluoromethoxy, (C₁-C₄)-alkoxy, hydroxycarbonyl, (C₁-C₄)-alkoxycarbonyl and amino,
      and in which
      Q is a bond or (C₁-C₄)-alkanediyl,
      R⁸ is —(C═O)ᵣ—OR⁹, —(C═O)ᵣ—NR⁹R¹⁰, —C(═S)—NR⁹R¹⁰, —NR⁹—(C═O)—R¹², —NR⁹—(C═O)—NR¹⁰R¹¹, —NR⁹—SO₂—NR¹⁰R¹¹, —NR⁹—SO₂—R¹², —S(O)ₛ—R¹², —SO₂—NR⁹R¹⁰, 4- to 7-membered heterocyclyl, phenyl or 5- or 6-membered heteroaryl,
         in which
         r is the number 0 or 1,
         s is the number 0, 1 or 2,
         R⁹, R¹⁰ and R¹¹ independently of one another are each hydrogen, (C₁-C₆)-alkyl, (C₃-C₈)-cycloalkyl, 4- to 7-membered heterocyclyl, phenyl or 5- or 6-membered heteroaryl,
         or
         R⁹ and R¹⁰ together with the atom(s) to which they are respectively attached form a 4- to 7-membered heterocycle,
            in which the 4- to 7-membered heterocycle for its part may be substituted by 1 or 2 substituents independently of one another selected from the group consisting of cyano, trifluoromethyl, (C₁-C₆)-alkyl, hydroxy, oxo, (C₁-C₆)-alkoxy, trifluoromethoxy, (C₁-C₆)-alkoxycarbonyl, amino, mono-(C₁-C₆)-alkylamino and di-(C₁-C₆)-alkylamino,
         or
         R¹⁰ and R¹¹ together with the atom(s) to which they are respectively attached form a 4- to 7-membered heterocycle,
            in which the 4- to 7-membered heterocycle for its part may be substituted by 1 or 2 substituents independently of one another selected from the group consisting of cyano, trifluoromethyl, (C₁-C₆)-alkyl, hydroxy, oxo, (C₁-C₆)-alkoxy, trifluoromethoxy, (C₁-C₆)-alkoxycarbonyl, amino, mono-(C₁-C₆)-alkylamino and di-(C₁-C₆)-alkylamino,
         R¹² is (C₁-C₆)-alkyl or (C₃-C₇)-cycloalkyl,
         or
         R⁹ and R¹² together with the atom(s) to which they are respectively attached form a 4- to 7-membered heterocycle,
            in which the 4- to 7-membered heterocycle for its part may be substituted by 1 or 2 substituents independently of one another selected from the group consisting of cyano, trifluoromethyl, (C₁-C₆)-alkyl, hydroxy, oxo, (C₁-C₆)-alkoxy, trifluoromethoxy, (C₁-C₆)-alkoxycarbonyl, amino, mono-(C₁-C₆)-alkylamino and di-(C₁-C₆)-alkylamino,
         and
         in which 4- to 7-membered heterocyclyl, phenyl and 5- or 6-membered heteroaryl for their part may be substituted by 1 to 3 substituents independently of one another selected from the group consisting of halogen, cyano, difluoromethyl, trifluoromethyl, (C₁-C₆)-alkyl, (C₃-C₇)-cycloalkyl, hydroxy, oxo, thioxo and (C₁-C₄)-alkoxy,
            and
            in which the aforementioned (C₁-C₄)-alkyl, (C₁-C₆)-alkyl, (C₃-C₈)-cycloalkyl and 4- to 7-membered heterocyclyl groups, unless stated otherwise, may each independently of one another additionally be substituted by 1 to 3 substituents independently of one another selected from the group consisting of fluorine, difluoromethyl, trifluoromethyl, (C₁-C₆)-alkyl, (C₃-C₇)-cycloalkyl, hydroxy, difluoromethoxy, trifluoromethoxy, (C₁-C₄)-alkoxy, hydroxycarbonyl, (C₁-C₄)-alkoxycarbonyl, amino, phenyl, 4- to 7-membered heterocyclyl and 5- or 6-membered heteroaryl,
   or
   R^{4A} and R^{4B} together with the carbon atom to which they are bonded form a (C₂-C₄)-alkenyl group, an oxo group, a 3- to 6-membered carbocycle or a 4- to 7-membered heterocycle,
      in which the 3- to 6-membered carbocycle and the 4- to 7-membered heterocycle may be substituted by 1 or 2 substituents selected independently from the group of fluorine and (C₁-C₄)-alkyl,
   R^{5A} is hydrogen, fluorine, (C₁-C₄)-alkyl, (C₁-C₄)-alkoxycarbonyl or hydroxy, R^{5B} is hydrogen, fluorine, (C₁-C₄)-alkyl or trifluoromethyl,
M is CH or N,
R¹ is (C₁-C₆)-alkyl, (C₃-C₈)-cycloalkylmethyl, benzyl or 5- or 6-membered heteroarylmethyl,
   where (C₁-C₆)-alkyl is substituted by a substituent selected from the group consisting of difluoromethyl and trifluoromethyl,
   where (C₁-C₆)-alkyl may be substituted by 1 to 3 fluorine substituents,
   where (C₃-C₈)-cycloalkylmethyl may be substituted by 1 or 2 substituents independently of one another selected from the group consisting of fluorine, methyl and methoxy,
   where benzyl is substituted by 1 to 3 fluorine substituents,
   and
   where 5- and 6-membered heteroarylmethyl may be substituted by 1 or 2 fluorine substituents,
R² is hydrogen, cyano, halogen, difluoromethyl, trifluoromethyl, (C₁-C₄)-alkyl or (C₃-C₇)-cycloalkyl,
R⁶ is hydrogen, cyano, halogen, difluoromethyl, trifluoromethyl, (C₁-C₄)-alkyl or (C₃-C₇)-cycloalkyl,
R⁷ is hydrogen, cyano, difluoromethyl, trifluoromethyl, (C₁-C₄)-alkyl or (C₃-C₇)-cycloalkyl,
   and the N-oxides, salts, solvates, salts of the N-oxides and solvates of the N-oxides and salts thereof.

Compounds according to the invention are the compounds of the formula (I) and their salts, solvates and solvates of the salts, the compounds, comprised by formula (I), of the formulae mentioned below and their salts, solvates and solvates of the salts and the compounds comprised by formula (I), mentioned below as embodiments, and their salts, solvates and solvates of the salts, if the compounds, comprised by formula (I), mentioned below are not already salts, solvates and solvates of the salts.

Compounds according to the invention are likewise N-oxides of the compounds of the formula (I) and the salts, solvates and solvates of the salts thereof.

Preferred salts in the context of the present invention are physiologically acceptable salts of the compounds according to the invention. Also encompassed are salts which are not themselves suitable for pharmaceutical applications but can be used, for example, for isolation or purification of the compounds according to the invention.

Physiologically acceptable salts of the compounds according to the invention include acid addition salts of mineral acids, carboxylic acids and sulfonic acids, for example salts of hydrochloric acid, hydrobromic acid, sulfuric acid, phosphoric acid, methanesulfonic acid, ethanesulfonic acid, toluenesulfonic acid, benzenesulfonic acid, naphthalenedisulfonic acid, formic acid, acetic acid, trifluoroacetic acid, propionic acid, lactic acid, tartaric acid, malic acid, citric acid, fumaric acid, maleic acid and benzoic acid.

Physiologically acceptable salts of the compounds according to the invention also include salts of conventional bases, by way of example and with preference alkali metal salts (e.g. sodium and potassium salts), alkaline earth metal salts (e.g. calcium and magnesium salts) and ammonium salts derived from ammonia or organic amines having 1 to 16 carbon atoms, by way of example and with preference ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine and N-methylpiperidine.

In the context of the invention, solvates refer to those forms of the compounds according to the invention which, in the solid or liquid state, form a complex by coordination with solvent molecules. Hydrates are a specific form of solvates in which the coordination is with water. Solvates preferred in the context of the present invention are hydrates.

The compounds according to the invention may, depending on their structure, exist in different stereoisomeric forms, i.e. in the form of configurational isomers or else optionally as conformational isomers (enantiomers and/or diastereomers, including those in the case of atropisomers). The present invention therefore encompasses the enantiomers and diastereomers, and the respective mixtures thereof. The stereoisomerically uniform constituents can be isolated from such mixtures of enantiomers and/or diastereomers in a known manner; chromatography processes are preferably used for this, in particular HPLC chromatography on an achiral or chiral phase.

Where the compounds according to the invention can occur in tautomeric forms, the present invention encompasses all the tautomeric forms.

The present invention also encompasses all suitable isotopic variants of the compounds according to the invention. An isotopic variant of a compound according to the invention is understood here to mean a compound in which at least one atom within the compound according to the invention has been exchanged for another atom of the same atomic number, but with a different atomic mass than that which occurs usually or predominantly in nature. Examples of isotopes which can be incorporated into a compound according to the invention are those of hydrogen, carbon, nitrogen, oxygen, phosphorus, sulfur, fluorine, chlorine, bromine and iodine, such as ²H (deuterium), ³H (tritium), ¹³C, ¹⁴C, ¹⁵N, ¹⁷O, ¹⁸O, ³²F, ³³F, ³³S, ³⁴S, ³⁵S, ³⁶S, ¹⁸F, ³⁶Cl, ⁸²Br, ¹²³I, ¹²⁴I, ¹²⁹I and ¹³¹I . Particular isotopic variants of a compound according to the invention, such as, more particularly, those in which one or more radioactive isotopes have been incorporated, may be of benefit, for example, for the study of the mechanism of action or of the active compound distribution in the body; due to the comparative ease of preparability and detectability, compounds labeled particularly with ³H or ¹⁴C isotopes are suitable for this purpose. Furthermore, the incorporation of isotopes, for example of deuterium, can lead to particular therapeutic advantages as a consequence of greater metabolic stability of the compound, for example an extension of the half-life in the body or a reduction in the active dose required; such modifications of the compounds according to the invention may therefore, in some cases, also constitute a preferred embodiment of the present invention. Isotopic variants of the compounds according to the invention can be prepared by the processes known to those skilled in the art, for example by the methods described below and the procedures described in the working examples, by using corresponding isotopic modifications of the respective reagents and/or starting compounds.

Moreover, the present invention also encompasses prodrugs of the compounds according to the invention. Here, the term “prodrugs” refers to compounds which for their part can be biologically active or inactive, but are converted (for example metabolically or hydrolytically) into compounds according to the invention during their dwell time in the body.

In the formula of the group which may represent L, the end point of the line marked by the symbol # or ## does not represent a carbon atom or a CH₂ group, but is part of the bond to the respective atom to which L is attached.

The compounds of the formula (I-1) form a sub-group of the compounds of the formula (I) in which R⁶ and R⁷ are hydrogen.

In the context of the present invention, the substituents, unless specified otherwise, are each defined as follows:

Alkyl in the context of the invention is a straight-chain or branched alkyl radical having the number of carbon atoms specified in each case. The following may be mentioned by way of example and by way of preference: methyl, ethyl, n-propyl, isopropyl, n-butyl, isobutyl, 1-methylpropyl, tert-butyl, n-pentyl, isopentyl, 1-ethylpropyl, 1-methylbutyl, 2-methylbutyl, 3-methylbutyl, n-hexyl, 1-methylpentyl, 2-methylpentyl, 3-methylpentyl, 4-methylpentyl, 3,3-dimethylbutyl, 1-ethylbutyl and 2-ethylbutyl.

Cycloalkyl or carbocycle in the context of the invention is a monocyclic saturated alkyl radical having the number of carbon atoms specified in each case. The following may be mentioned by way of example and by way of preference: cyclopropyl, cyclobutyl, cyclopentyl, cyclohexyl and cycloheptyl.

Alkanediyl in the context of the invention is a straight-chain or branched divalent alkyl radical having 1 to 4 carbon atoms. The following may be mentioned by way of example and by way of preference: methylene, ethane-1,2-diyl, ethane-1,1-diyl, propane-1,3-diyl, propane-1,1-diyl, propane-1,2-diyl, propane-2,2-diyl, butane-1,4-diyl, butane-1,2-diyl, butane-1,3-diyl and butane-2,3-diyl.

Alkenyl in the context of the invention is a straight-chain or branched alkenyl radical having 2 to 4 carbon atoms and a double bond. The following may be mentioned by way of example and by way of preference: vinyl, allyl, isopropenyl and n-but-2-en-1-yl.

Alkynyl in the context of the invention is a linear or branched alkynyl radical having 2 to 4 carbon atoms and one triple bond. The following may be mentioned by way of example and by way of preference: ethynyl, n-prop-1-yn-1-yl, n-prop-2-yn-1-yl, n-but-2-yn-1-yl and n-but-3-yn-1-yl.

Alkoxy in the context of the invention is a straight-chain or branched alkoxy radical having 1 to 6 or 1 to 4 carbon atoms. The following may be mentioned by way of example: methoxy, ethoxy, n-propoxy, isopropoxy, 1-methylpropoxy, n-butoxy, isobutoxy, tert-butoxy, n-pentoxy, isopentoxy, 1-ethylpropoxy, 1-methylbutoxy, 2-methylbutoxy, 3-methylbutoxy and n-hexoxy. Preference is given to a straight-chain or branched alkoxy radical having 1 to 4 carbon atoms. The following may be mentioned by way of example and by way of preference: methoxy, ethoxy, n-propoxy, isopropoxy, 1-methylpropoxy, n-butoxy, isobutoxy, tert-butoxy.

Alkoxycarbonyl in the context of the invention is a straight-chain or branched alkoxy radical having 1 to 6 or 1 to 4 carbon atoms and a carbonyl group attached to the oxygen. The following may be mentioned by way of example and by way of preference: methoxycarbonyl, ethoxycarbonyl, n-propoxycarbonyl, isopropoxycarbonyl and tert-butoxycarbonyl.

Alkoxycarbonylamino in the context of the invention is an amino group having a straight-chain or branched alkoxycarbonyl substituent which has 1 to 4 carbon atoms in the alkyl chain and is attached via the carbonyl group to the nitrogen atom. The following may be mentioned by way of example and by way of preference: methoxycarbonylamino, ethoxycarbonylamino, prop oxycarbonylamino, n-butoxycarbonylamino, isobutoxycarbonylamino and tert-butoxycarbonylamino.

Monoalkylamino in the context of the invention is an amino group having a linear or branched alkyl substituent having 1 to 6 carbon atoms. The following may be mentioned by way of example and by way of preference: methylamino, ethylamino, n-propylamino, isopropylamino and tert-butylamino.

Dialkylamino in the context of the invention is an amino group having two identical or different, linear or branched alkyl substituents each having 1 to 6 carbon atoms. The following may be mentioned by way of example and by way of preference: N,N-dimethylamino, N,N-diethylamino, N-ethyl-N-methylamino, N-methyl-N-n-propylamino, N-isopropyl-N-n-propylamino, N-tert-butyl-N-methylamino, N-ethyl-N-n-pentylamino and N-n-hexyl-N-methylamino.

Heterocyclyl or heterocycle in the context of the invention is a saturated heterocycle which has a total of 4 to 7 ring atoms and contains one or two ring heteroatom from the group consisting of N, O, S, SO and SO₂. The following may be mentioned by way of example: azetidinyl, oxetanyl, pyrrolidinyl, pyrazolidinyl, imidazolinyl, tetrahydrofuranyl, piperidinyl, piperazinyl, tetrahydropyranyl, morpholinyl, thiomorpholinyl and dioxidothiomorpholinyl. Preference is given to azetidinyl, oxetanyl, pyrrolidinyl, tetrahydrofuranyl, piperidinyl, tetrahydropyranyl and morpholinyl.

5- or 6-membered heteroaryl in the context of the invention is a monocyclic aromatic heterocycle (heteroaromatic) which has a total of 5 or 6 ring atoms, contains up to three identical or different ring heteroatoms from the group consisting of N, O and S and is attached via a ring carbon atom or optionally via a ring nitrogen atom. The following may be mentioned by way of example and by way of preference: furyl, pyrrolyl, thienyl, pyrazolyl, imidazolyl, thiazolyl, oxazolyl, isoxazolyl, isothiazolyl, triazolyl, oxadiazolyl, thiadiazolyl, pyridyl, pyrimidinyl, pyridazinyl, pyrazinyl and triazinyl. Preference is given to: pyrazolyl, oxazolyl, thiazolyl, triazolyl, oxadiazolyl, thiadiazolyl, pyridyl and pyrimidinyl.

Halogen in the context of the invention is fluorine, chlorine, bromine and iodine. Preference is given to bromine and iodine.

An oxo group in the context of the invention is an oxygen atom bonded via a double bond to a carbon atom.

A thiooxo group in the context of the invention is a sulfur atom bonded via a double bond to a carbon atom.

When radicals in the compounds according to the invention are substituted, the radicals, unless specified otherwise, may be mono- or polysubstituted. In the context of the present invention, all radicals which occur more than once are defined independently of one another. Substitution by one, two or three identical or different substituents is preferred.

In the context of the present invention, the term “treatment” or “treating” includes inhibition, retardation, checking, alleviating, attenuating, restricting, reducing, suppressing, repelling or healing of a disease, a condition, a disorder, an injury or a health problem, or the development, the course or the progress of such states and/or the symptoms of such states. The term “therapy” is understood here to be synonymous with the term “treatment”.

The terms “prevention”, “prophylaxis” or “preclusion” are used synonymously in the context of the present invention and refer to the avoidance or reduction of the risk of contracting, experiencing, suffering from or having a disease, a condition, a disorder, an injury or a health problem, or a development or advancement of such states and/or the symptoms of such states.

The treatment or prevention of a disease, a condition, a disorder, an injury or a health problem may be partial or complete.

Preference is given in the context of the present invention to compounds of the formula (I) in which
A is nitrogen or CR³,
   where
   R³ is hydrogen, deuterium, fluorine, iodine, difluoromethyl, trifluoromethyl, (C₁-C₄)-alkyl, vinyl, allyl, ethynyl, cyclopropyl, cyclobutyl, hydroxy, pyrazolyl or pyridyl,
      where (C₁-C₄)-alkyl, vinyl, allyl, ethynyl and pyridyl may be substituted by 1 or 2 substituents independently of one another selected from the group consisting of methyl, cyclopropyl and cyclobutyl,
L is a #-CR^{4A}R^{4B}—(CR^{5A}R^{5B})ₚ-## group
   where
   # is the attachment site to the carbonyl group,
   ## is the point of attachment to the pyrimidine or triazine ring,
   p is a number 0 or 1,
   R^{4A} is hydrogen, fluorine, methyl, ethyl, hydroxy or amino,
   R^{4B} is hydrogen, fluorine, difluoromethyl, trifluoromethyl, (C₁-C₄)-alkyl, methoxycarbonylamino, cyano, cyclopropyl, cyclobutyl, cyclopentyl, phenyl or a group of the formula -Q-R⁸,
      in which (C₁-C₄)-alkyl may be substituted by 1 to 3 substituents independently of one another selected from the group consisting of fluorine, cyano, trifluoromethyl, cyclopropyl, cyclobutyl, cyclopentyl, hydroxy, difluoromethoxy, trifluoromethoxy, methoxy, ethoxy, hydroxycarbonyl, methoxycarbonyl, ethoxycarbonyl and amino,
      and in which
      Q is a bond or methylene,
      R⁸ is —(C═O)ᵣ—NR⁹R¹⁰, —C(═S)—NR⁹R¹⁰, oxadiazolonyl, oxadiazolthionyl, phenyl, oxazolyl, thiazolyl, pyrazolyl, triazolyl, oxadiazolyl, thiadiazolyl, pyridyl, pyrimidinyl or pyrazinyl,
         in which
         r is the number 0 or 1,
         R⁹ and R¹⁰ are each independently of one another hydrogen, methyl, ethyl, isopropyl, cyclopropyl, cyclobutyl, cyclopentyl, oxetanyl, azetidinyl, tetrahydrofuranyl, pyrrolidinyl, tetrahydropyranyl, piperidinyl, piperazinyl, morpholinyl, phenyl, pyrazolyl or pyridyl,
            in which methyl, ethyl and isopropyl may additionally be substituted by 1 or 2 substituents independently of one another selected from the group consisting of fluorine, difluoromethyl, trifluoromethyl, cyclopropyl, cyclobutyl, cyclopentyl, hydroxy, difluoromethoxy, trifluoromethoxy, methoxy, ethoxy, hydroxycarbonyl, methoxycarbonyl, ethoxycarbonyl and amino,
         and
         in which oxadiazolonyl, oxadiazolthionyl, phenyl, oxazolyl, thiazolyl, pyrazolyl, triazolyl, oxadiazolyl, thiadiazolyl, pyridyl, pyrimidinyl and pyrazinyl for their part may be substituted by 1 or 2 substituents independently of one another selected from the group consisting of fluorine, chlorine, cyano, difluoromethyl, trifluoromethyl, methyl, ethyl, isopropyl, 2,2,2-trifluoroethyl, 1,1,2,2,2-pentafluoroethyl, cyclopropyl, cyclobutyl, cyclopropylmethyl, cyclobutylmethyl, hydroxy, methoxy and ethoxy,
   or
   R^{4A} and R^{4B} together with the carbon atom to which they are attached form a cyclopropyl, cyclobutyl, cyclopentyl, azetidinyl, tetrahydrofuranyl, pyrrolidinyl or tetrahydropyranyl ring,
      in which the cyclopropyl, cyclobutyl, cyclopentyl, azetidinyl, tetrahydrofuranyl, pyrrolidinyl and tetrahydropyranyl ring may be substituted by 1 or 2 substituents independently of one another selected from the group consisting of fluorine and methyl,
   R^{5A} is hydrogen, fluorine, methyl, ethyl or hydroxy,
   R^{5B} is hydrogen, fluorine, methyl, ethyl or trifluoromethyl,
M represents CH or N,
R¹ is 3,3,3-trifluoroprop-1-yl, 2,2,3,3,3-pentafluoroprop-1-yl, 4,4,4-trifluorobut-1-yl, 3,3,4,4-tetrafluorobut-1-yl, 3,3,4,4,4-pentafluorobut-1-yl(C₃-C₆)-cycloalkylmethyl, benzyl, thienylmethyl, pyridylmethyl, pyrimidinylmethyl, pyrazinylmethyl or pyridazinylmethyl,
   where benzyl is substituted by 1 to 3 fluorine substituents,
   and
   where (C₃-C₆)-cycloalkylmethyl, thienylmethyl, pyridylmethyl, pyrimidinylmethyl, pyrazinylmethyl and pyridazinylmethyl may be substituted by 1 or 2 fluorine substituents,
R² is hydrogen, fluorine or chlorine,
R⁶ is hydrogen, fluorine, chlorine or methyl,
R⁷ is hydrogen or methyl,
   and the salts, solvates and solvates of the salts thereof.

Preference is also given in the context of the present invention to compounds of the formula (I) in which
A is nitrogen or CR³,
   where
   R³ is hydrogen,
L is a #-CR^{4A}R^{4B}—(CR^{5A}R^{5B})ₚ-## group
   where
   # is the attachment site to the carbonyl group,
   ## is the point of attachment to the pyrimidine or triazine ring,
   p is a number 0,
   R^{4A} is hydrogen, fluorine, methyl, ethyl, hydroxy or amino,
   R^{4B} is hydrogen, fluorine, difluoromethyl, trifluoromethyl, methyl, ethyl, methoxycarbonylamino, cyclopropyl, cyclobutyl, cyclopentyl or a group of the formula -Q-R⁸,
      in which methyl and ethyl may be substituted by 1 to 3 substituents independently of one another selected from the group consisting of fluorine, cyano, trifluoromethyl, cyclopropyl, cyclobutyl, hydroxy, difluoromethoxy, trifluoromethoxy, methoxy, ethoxy, hydroxycarbonyl, methoxycarbonyl, ethoxycarbonyl and amino,
      and in which
      Q is a bond,
      R⁸ is —(C═O)ᵣ—NR⁹R¹⁰, phenyl, thiazolyl, triazolyl, oxadiazolyl, thiadiazolyl or pyrimidinyl,
         in which
         r is the number 1,
         R⁹ and R¹⁰ independently of one another are each hydrogen or cyclopropyl,
         and
         in which phenyl, thiazolyl, triazolyl, oxadiazolyl, thiadiazolyl and pyrimidinyl for their part may be substituted 1 or 2 substituents independently of one another selected from the group consisting of fluorine, difluoromethyl, trifluoromethyl, methyl, ethyl, isopropyl, 2,2,2-trifluoroethyl, 1,1,2,2,2-pentafluoroethyl, cyclopropyl, cyclobutyl, cyclopropylmethyl and cyclobutylmethyl,
   or
   R^{4A} and R^{4B} together with the carbon atom to which they are attached form a cyclopropyl, cyclobutyl, cyclopentyl, azetidinyl, tetrahydrofuranyl, pyrrolidinyl or tetrahydropyranyl ring,
      in which the cyclopropyl, cyclobutyl, cyclopentyl, azetidinyl, tetrahydrofuranyl, pyrrolidinyl and tetrahydropyranyl ring may be substituted by 1 or 2 substituents independently of one another selected from the group consisting of fluorine and methyl,
M is CH or N,
R¹ is 4,4,4-trifluorobut-1-yl, 3,3,4,4-tetrafluorobut-1-yl, 3,3,4,4,4-pentafluorobut-1-yl or benzyl,
   where benzyl is substituted by 1 to 3 fluorine substituents,
R² is fluorine or chlorine if M is CH,
or
R² is hydrogen if M is N,
R⁶ is hydrogen,
R⁷ represents hydrogen,
   and the salts, solvates and solvates of the salts thereof.

Particular preference is given in the context of the present invention to compounds of the formula (I) in which
A is nitrogen or CR³,
   where
   R³ represents hydrogen,
L is a #-CR^{4A}R^{4B}—(CR^{5A}R^{5B})ₚ-## group
   where
   # is the attachment site to the carbonyl group,
   ## is the point of attachment to the pyrimidine or triazine ring,
   p is a number 0,
   R^{4A} is hydrogen, fluorine, methyl or hydroxy,
   R^{4B} is hydrogen, fluorine, trifluoromethyl, 2,2,2-trifluoroethyl or methyl,
M is CH or N,
R¹ is 4,4,4-trifluorobut-1-yl, 3,3,4,4-tetrafluorobut-1-yl, 3,3,4,4,4-pentafluorobut-1-yl or benzyl,
   where benzyl is substituted by 1 to 3 fluorine substituents,
R² is fluorine or chlorine if M is CH,
or
R² is hydrogen if M is N,
R⁶ is hydrogen,
R⁷ represents hydrogen,
   and the salts, solvates and solvates of the salts thereof.

Preference is given in the context of the present invention to compounds of the formula (I-1) in which

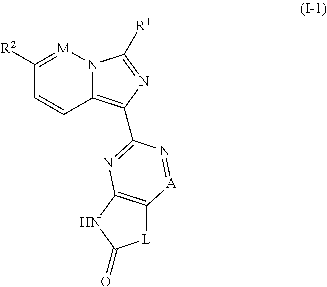

in which
A is nitrogen or CR³,
   where
   R³ is hydrogen, deuterium, fluorine, difluoromethyl, trifluoromethyl, (C₁-C₄)-alkyl, cyclopropyl or cyclobutyl,
L is a #-CR^{4A}R^{4B}—(CR^{5A}R^{5B})ₚ-## group
   where
   # is the attachment site to the carbonyl group,
   ## is the point of attachment to the pyrimidine or triazine ring,
   p is a number 0, 1 or 2,
   R^{4A} is hydrogen, fluorine, (C₁-C₄)-alkyl, hydroxy or amino,
   R^{4B} is hydrogen, fluorine, (C₁-C₄)-alkyl, trifluoromethyl, (C₁-C₄)-alkoxycarbonylamino or phenyl,
      in which (C₁-C₄)-alkyl may be substituted by 1 to 3 substituents independently of one another selected from the group consisting of fluorine, trifluoromethyl, hydroxy, hydroxycarbonyl and (C₁-C₄)-alkoxycarbonyl,
   or
   R^{4A} and R^{4B} together with the carbon atom to which they are attached form an oxo group, a 3- to 6-membered carbocycle or a 4- to 6-membered heterocycle, in which the 3- to 6-membered carbocycle and the 4- to 6-membered heterocycle may be substituted by 1 or 2 substituents independently of one another selected from the group consisting of fluorine and (C₁-C₄)-alkyl,
   or
   R^{4A} and R^{4B} together with the carbon atom to which they are attached form a (C₂-C₄)-alkenyl group,
   R^{5A} is hydrogen, fluorine, (C₁-C₄)-alkyl or hydroxy,
   R^{5B} is hydrogen, fluorine, (C₁-C₄)-alkyl or trifluoromethyl,
M is CH or N,
R¹ represents (C₁-C₆)-alkyl or benzyl,
   where (C₁-C₆)-alkyl is substituted by one trifluoromethyl substituent,
   where (C₁-C₆)-alkyl may be substituted by 1 to 3 fluorine substituents,
   and
   where benzyl is substituted by 1 to 3 fluorine substituents,
R² is hydrogen, fluorine or chlorine,
   and the N-oxides, salts, solvates, salts of the N-oxides and solvates of the N-oxides and salts thereof.

Preference is given in the context of the present invention to compounds of the formula (I-1) in which
A is nitrogen or CR³,
   where
   R³ is hydrogen, fluorine, difluoromethyl, trifluoromethyl, methyl, ethyl or cyclopropyl,
L is a #-CR^{4A}R^{4B}—(CR^{5A}R^{5B})ₚ-## group
   where
   # is the attachment site to the carbonyl group,
   ## is the point of attachment to the pyrimidine or triazine ring,
   p is a number 0 or 1,
   R^{4A} is hydrogen, fluorine, methyl, ethyl or hydroxy,
   R^{4B} is hydrogen, fluorine, methyl, ethyl, trifluoromethyl, methoxycarbonylamino or phenyl,
      in which methyl and ethyl may be substituted by 1 to 3 substituents independently of one another selected from the group consisting of fluorine, trifluoromethyl and hydroxy,
   or
   R^{4A} and R^{4B} together with the carbon atom to which they are attached form a cyclopropyl, cyclobutyl, cyclopentyl, azetidinyl, pyrrolidinyl, tetrahydrofuranyl, piperidinyl or tetrahydropyranyl ring,
      in which the cyclopropyl, cyclobutyl, cyclopentyl, azetidinyl, pyrrolidinyl, tetrahydrofuranyl, piperidinyl and tetrahydropyranyl ring may be substituted by 1 or 2 substituents independently of one another selected from the group consisting of fluorine and methyl,
   R^{5A} is hydrogen, fluorine, methyl, ethyl or hydroxy,
   R^{5B} is hydrogen, fluorine, methyl, ethyl or trifluoromethyl,
M is CH,
R¹ is 2,2,2-trifluoroethyl, 3,3,3-trifluoroprop-1-yl, 4,4,4-trifluorobut-1-yl, 3,3,4,4,4-pentafluorobut-1-yl or benzyl,
   where benzyl is substituted by 1 to 3 fluorine substituents,
R² is fluorine or chlorine,
   and the salts, solvates and solvates of the salts thereof.

Preference is also given in the context of the present invention to compounds of the formula (I-1) in which
A is nitrogen or CR³,
   where
   R³ is hydrogen, fluorine, difluoromethyl, trifluoromethyl, methyl, ethyl or cyclopropyl,
L is a #-CR^{4A}R^{4B}—(CR^{5A}R^{5B})ₚ-## group
   where
   # is the attachment site to the carbonyl group,
   ## is the point of attachment to the pyrimidine or triazine ring,
   p is a number 0 or 1,
   R^{4A} is hydrogen, fluorine, methyl, ethyl or hydroxy,
   R^{4B} is hydrogen, fluorine, methyl, ethyl, trifluoromethyl, methoxycarbonylamino or phenyl,
      in which methyl and ethyl may be substituted by 1 to 3 substituents independently of one another selected from the group consisting of fluorine, trifluoromethyl and hydroxy,
   or
   R^{4A} and R^{4B} together with the carbon atom to which they are attached form a cyclopropyl, cyclobutyl, cyclopentyl, azetidinyl, pyrrolidinyl, tetrahydrofuranyl, piperidinyl or tetrahydropyranyl ring,
      in which the cyclopropyl, cyclobutyl, cyclopentyl, azetidinyl, pyrrolidinyl, tetrahydrofuranyl, piperidinyl and tetrahydropyranyl ring may be substituted by 1 or 2 substituents independently of one another selected from the group consisting of fluorine and methyl,
   R^{5A} is hydrogen, fluorine, methyl, ethyl or hydroxy,
   R^{5B} is hydrogen, fluorine, methyl, ethyl or trifluoromethyl,
M is N,
R¹ is 2,2,2-trifluoroethyl, 3,3,3-trifluoroprop-1-yl, 4,4,4-trifluorobut-1-yl, 3,3,4,4,4-pentafluorobut-1-yl or benzyl,
   where benzyl is substituted by 1 to 3 fluorine substituents,
R² is hydrogen,
   and the salts, solvates and solvates of the salts thereof.

Particular preference is given in the context of the present invention to compounds of the formula (I-1) in which

A is nitrogen or CR³,
where
R³ represents hydrogen,
   L is a #-CR^{4A}R^{4B}—(CR^{5A}R^{5B})ₚ-## group
where
# is the attachment site to the carbonyl group,
## is the point of attachment to the pyrimidine or triazine ring,
p is a number 0,
R^{4A} is hydrogen, fluorine, methyl or hydroxy,
R^{4B} is hydrogen, fluorine, methyl or trifluoromethyl,
or
R^{4A} and R^{4B} together with the carbon atom to which they are attached form a cyclopropyl or cyclobutyl ring,
   in which the cyclopropyl and the cyclobutyl ring may be substituted by 1 or 2 substituents independently of one another selected from the group consisting of fluorine and methyl,

### M is CH,

R¹ is 3,3,4,4,4-pentafluorobut-1yl, 2-fluorobenzyl or 2,3,6-trifluorobenzyl,
R² is fluorine or chlorine,
and the salts, solvates and solvates of the salts thereof.

Particular preference is given in the context of the present invention to compounds of the formula (I-1) in which

A is nitrogen or CR³,
where
R³ represents hydrogen,
   L is a #-CR^{4A}R^{4B}—(CR^{5A}R^{5B})ₚ-## group
where
# is the attachment site to the carbonyl group,
## is the point of attachment to the pyrimidine or triazine ring,
p is a number 0,
R^{4A} is hydrogen, fluorine, methyl or hydroxy,
R^{4B} is hydrogen, fluorine, methyl or trifluoromethyl,
or
R^{4A} and R^{4B} together with the carbon atom to which they are attached form a cyclopropyl or cyclobutyl ring,
   in which the cyclopropyl and the cyclobutyl ring may be substituted by 1 or 2 substituents independently of one another selected from the group consisting of fluorine and methyl,

### M is N,

R¹ is 3,3,4,4,4-pentafluorobut-1yl, 2-fluorobenzyl or 2,3,6-trifluorobenzyl,
R² is hydrogen,
and the salts, solvates and solvates of the salts thereof.

Preference is also given in the context of the present invention to compounds of the formula (I) and (I-1) in which

A is nitrogen,
and the salts, solvates and solvates of the salts thereof.

Preference is also given in the context of the present invention to compounds of the formula (I) and (I-1) in which

### A is CR³,

where
R³ represents hydrogen,
   and the salts, solvates and solvates of the salts thereof.

Preference is also given in the context of the present invention to compounds of the formula (I) and (I-1) in which

### M is N,

R² is hydrogen,
and the salts, solvates and solvates of the salts thereof.

Preference is also given in the context of the present invention to compounds of the formula (I) and (I-1) in which

### M is CH,

R² is fluorine or chlorine,
and the salts, solvates and solvates of the salts thereof.

Preference is also given in the context of the present invention to compounds of the formula (I) and (I-1) in which

L is a #-CR^{4A}R^{4B}—(CR^{5A}R^{5B})ₚ-## group
where
# is the attachment site to the carbonyl group,
## is the point of attachment to the pyrimidine or triazine ring,
p is a number 0,
R^{4A} is hydrogen, fluorine, methyl or hydroxy,
R^{4B} is hydrogen, fluorine, methyl or trifluoromethyl,
   and the salts, solvates and solvates of the salts thereof.

Preference is also given in the context of the present invention to compounds of the formula (I) and (I-1) in which

L is a #-CR^{4A}R^{4B}—(CR^{5A}R^{5B})ₚ-## group
where
# is the attachment site to the carbonyl group,
## is the point of attachment to the pyrimidine or triazine ring,
p is a number 0,
R^{4A} is hydrogen, fluorine, methyl or hydroxy,
R^{4B} is hydrogen, fluorine, methyl, trifluoromethyl, 2,2,2-trifluoroethyl or 1,1,2,2,2-pentafluoroethyl,
   and the salts, solvates and solvates of the salts thereof.

Preference is also given in the context of the present invention to compounds of the formula (I) and (I-1) in which

L is a #-CR^{4A}R^{4B}—(CR^{5A}R^{5B})ₚ-## group
where
# is the attachment site to the carbonyl group,
## is the point of attachment to the pyrimidine or triazine ring,
p is a number 0,
R^{4A} is hydrogen, fluorine, methyl or hydroxy,
R^{4B} is trifluoromethyl, 2,2,2-trifluoroethyl or 1,1,2,2,2-pentafluoroethyl,
   and the salts, solvates and solvates of the salts thereof.

Preference is also given in the context of the present invention to compounds of the formula (I) and (I-1) in which

L is a #-CR^{4A}R^{4B}—(CR^{5A}R^{5B})ₚ-## group
where
# is the attachment site to the carbonyl group,
## is the point of attachment to the pyrimidine or triazine ring,
p is a number 0,
R^{4A} is methyl,
R^{4B} is methyl,
   and the salts, solvates and solvates of the salts thereof.

Preference is also given in the context of the present invention to compounds of the formula (I) and (I-1) in which

L is a #-CR^{4A}R^{4B}—(CR^{5A}R^{5B})ₚ-## group
where
# is the attachment site to the carbonyl group,
## is the point of attachment to the pyrimidine or triazine ring,
p is a number 0,
R^{4A} and R^{4B} together with the carbon atom to which they are attached form a cyclopropyl, cyclobutyl, cyclopentyl, azetidinyl, pyrrolidinyl, tetrahydrofuranyl, piperidinyl or tetrahydropyranyl ring,
   and the salts, solvates and solvates of the salts thereof.

Preference is also given in the context of the present invention to compounds of the formula (I) in which

L is a #-CR^{4A}R^{4B}—(CR^{5A}R^{5B})ₚ-## group
where
# is the attachment site to the carbonyl group,
## is the point of attachment to the pyrimidine or triazine ring,
p is a number 0,
R^{4A} and R^{4B} together with the carbon atom to which they are attached form an azetidinyl, pyrrolidinyl, tetrahydrofuranyl, piperidinyl or tetrahydropyranyl ring,
   and the salts, solvates and solvates of the salts thereof.

Preference is also given in the context of the present invention to compounds of the formula (I) and (I-1) in which

R¹ is benzyl,
where benzyl is substituted by 1 to 3 fluorine substituents,
   and the salts, solvates and solvates of the salts thereof.

Preference is also given in the context of the present invention to compounds of the formula (I) and (I-1) in which

R¹ is 2-fluorobenzyl, 2,3-difluorobenzyl or 2,3,6-trifluorobenzyl,
and the salts, solvates and solvates of the salts thereof.

Preference is also given in the context of the present invention to compounds of the formula (I) and (I-1) in which
R¹ is 2,2,2-trifluoroethyl, 3,3,3-trifluoroprop-1-yl, 4,4,4-trifluorobut-1-yl or 3,3,4,4,4-pentafluorobut-1-yl,
   and the salts, solvates and solvates of the salts thereof.

Preference is also given in the context of the present invention to compounds of the formula (I) in which

### A is CR³,

where
R³ represents hydrogen,
L is a #-CR^{4A}R^{4B}—(CR^{5A}R^{5B})ₚ-## group
where
# is the attachment site to the carbonyl group,
## is the point of attachment to the pyrimidine ring,
p is a number 0,
R^{4A} is hydrogen, fluorine, methyl, ethyl, hydroxy or amino,
R^{4B} is hydrogen, fluorine, difluoromethyl, trifluoromethyl, (C₁-C₄)-alkyl, methoxycarbonylamino, cyano, cyclopropyl, cyclobutyl, cyclopentyl, phenyl or a group of the formula -Q-R⁸,
   in which (C₁-C₄)-alkyl may be substituted by 1 to 3 substituents independently of one another selected from the group consisting of fluorine, cyano, trifluoromethyl, cyclopropyl, cyclobutyl, cyclopentyl, hydroxy, difluoromethoxy, trifluoromethoxy, methoxy, ethoxy, hydroxycarbonyl, methoxycarbonyl, ethoxycarbonyl and amino,
   and in which
   Q is a bond or methylene,
   R⁸ is —(C═O)ᵣ—NR⁹R¹⁰, —C(═S)—NR⁹R¹⁰, oxadiazolonyl, oxadiazolthionyl, phenyl, oxazolyl, thiazolyl, pyrazolyl, triazolyl, oxadiazolyl, thiadiazolyl, pyridyl, pyrimidinyl or pyrazinyl,
      in which
      r is the number 0 or 1,
      R⁹ and R¹⁰ are each independently of one another hydrogen, methyl, ethyl, isopropyl, cyclopropyl, cyclobutyl, cyclopentyl, oxetanyl, azetidinyl, tetrahydropyranyl, pyrrolidinyl, tetrahydropyranyl, piperidinyl, piperazinyl, morpholinyl, phenyl, pyrazolyl or pyridyl,
         in which methyl, ethyl and isopropyl may additionally be substituted by 1 or 2 substituents independently of one another selected from the group consisting of fluorine, difluoromethyl, trifluoromethyl, cyclopropyl, cyclobutyl, cyclopentyl, hydroxy, difluoromethoxy, trifluoromethoxy, methoxy, ethoxy, hydroxycarbonyl,
      and
      in which oxadiazolonyl, oxadiazolthionyl, phenyl, oxazolyl, thiazolyl, pyrazolyl, triazolyl, oxadiazolyl, thiadiazolyl, pyridyl, pyrimidinyl and pyrazinyl for their part may be substituted by 1 or 2 substituents independently of one another selected from the group consisting of fluorine, chlorine, cyano, difluoromethyl, trifluoromethyl, methyl, ethyl, isopropyl, 2,2,2-trifluoroethyl, 1,1,2,2,2-pentafluoroethyl, cyclopropyl, cyclobutyl, cyclopropylmethyl, cyclobutylmethyl, hydroxy, methoxy and ethoxy,
         and the salts, solvates and solvates of the salts thereof.

Preference is also given in the context of the present invention to compounds of the formula (I) in which

### A is N,

L is a #-CR^{4A}R^{4B}—(CR^{5A}R^{5B})ₚ-## group
where
# is the attachment site to the carbonyl group,
## is the point of attachment to the triazine ring,
p is a number 0,
R^{4A} is hydrogen, fluorine, methyl, ethyl, hydroxy or amino,
R^{4B} is hydrogen, fluorine, difluoromethyl, trifluoromethyl, (C₁-C₄)-alkyl, methoxycarbonylamino, cyano, cyclopropyl, cyclobutyl, cyclopentyl, phenyl or a group of the formula -Q-R⁸,
   in which (C₁-C₄)-alkyl may be substituted by 1 to 3 substituents independently of one another selected from the group consisting of fluorine, cyano, trifluoromethyl, cyclopropyl, cyclobutyl, cyclopentyl, hydroxy, difluoromethoxy, trifluoromethoxy, methoxy, ethoxy, hydroxycarbonyl, methoxycarbonyl, ethoxycarbonyl and amino,
   and in which
   Q is a bond or methylene,
   R⁸ is —(C═O)ᵣ—NR⁹R¹⁰, —C(═S)—NR⁹R¹⁰, oxadiazolonyl, oxadiazolthionyl, phenyl, oxazolyl, thiazolyl, pyrazolyl, triazolyl, oxadiazolyl, thiadiazolyl, pyridyl, pyrimidinyl or pyrazinyl,
      in which
      r is the number 0 or 1,
      R⁹ and R¹⁰ are each independently of one another hydrogen, methyl, ethyl, isopropyl, cyclopropyl, cyclobutyl, cyclopentyl, oxetanyl, azetidinyl, tetrahydropyranyl, pyrrolidinyl, tetrahydropyranyl, piperidinyl, piperazinyl, morpholinyl, phenyl, pyrazolyl or pyridyl,
         in which methyl, ethyl and isopropyl may additionally be substituted by 1 or 2 substituents independently of one another selected from the group consisting of fluorine, difluoromethyl, trifluoromethyl, cyclopropyl, cyclobutyl, cyclopentyl, hydroxy, difluoromethoxy, trifluoromethoxy, methoxy, ethoxy, hydroxycarbonyl, methoxycarbonyl, ethoxycarbonyl and amino,
      and
      in which oxadiazolonyl, oxadiazolthionyl, phenyl, oxazolyl, thiazolyl, pyrazolyl, triazolyl, oxadiazolyl, thiadiazolyl, pyridyl, pyrimidinyl and pyrazinyl for their part may be substituted by 1 or 2 substituents independently of one another selected from the group consisting of fluorine, chlorine, cyano, difluoromethyl, trifluoromethyl, methyl, ethyl, isopropyl, 2,2,2-trifluoroethyl, 1,1,2,2,2-pentafluoroethyl, cyclopropyl, cyclobutyl, cyclopropylmethyl, cyclobutylmethyl, hydroxy, methoxy and ethoxy,
         and the salts, solvates and solvates of the salts thereof.

Preference is also given in the context of the present invention to compounds of the formula (I) in which

### A is CR³,

where
R³ represents hydrogen,
   L is a #-CR^{4A}R^{4B}—(CR^{5A}R^{5B})ₚ-## group
where
# is the attachment site to the carbonyl group,
## is the point of attachment to the pyrimidine ring,
p is a number 0,
R^{4A} is hydrogen, fluorine, methyl or hydroxy,
R^{4B} is hydrogen, fluorine, trifluoromethyl, 2,2,2-trifluoroethyl or methyl,
   and the salts, solvates and solvates of the salts thereof.

Preference is also given in the context of the present invention to compounds of the formula (I) in which

A is nitrogen,
L is a #-CR^{4A}R^{4B}—(CR^{5A}R^{5B})ₚ-## group
where
# is the attachment site to the carbonyl group,
## is the point of attachment to the triazine ring,
p is a number 0,
R^{4A} is hydrogen, fluorine, methyl or hydroxy,
R^{4B} is hydrogen, fluorine, trifluoromethyl, 2,2,2-trifluoroethyl or methyl,
   and the salts, solvates and solvates of the salts thereof.

Preference is also given in the context of the present invention to compounds of the formula (I) in which

### A is CR³,

where
R³ represents hydrogen,
   L is a #-CR^{4A}R^{4B}—(CR^{5A}R^{5B})ₚ-## group
where
# is the attachment site to the carbonyl group,
## is the point of attachment to the pyrimidine ring,
p is a number 0,
R^{4A} is hydrogen, fluorine, methyl, ethyl, hydroxy or amino,
R^{4B} is a group of the formula -Q-R⁸,
   in which
   Q is a bond,
   R⁸ is —(C═O)ᵣ—NR⁹R¹⁰, —C(═S)—NR⁹R¹⁰, oxadiazolonyl, oxadiazolthionyl, phenyl, oxazolyl, thiazolyl, pyrazolyl, triazolyl, oxadiazolyl, thiadiazolyl, pyridyl, pyrimidinyl or pyrazinyl,
      in which
      r is the number 0,
      R⁹ and R¹⁰ are each independently of one another hydrogen, methyl, ethyl, isopropyl, cyclopropyl, cyclobutyl, cyclopentyl, oxetanyl, azetidinyl, tetrahydrofuranyl, pyrrolidinyl, tetrahydropyranyl, piperidinyl, piperazinyl, morpholinyl, phenyl, pyrazolyl or pyridyl,
         in which methyl, ethyl and isopropyl for their part may be substituted by 1 or 2 substituents independently of one another selected from the group consisting of fluorine, difluoromethyl, trifluoromethyl, cyclopropyl, cyclobutyl, cyclopentyl, hydroxy, difluoromethoxy, trifluoromethoxy, methoxy, ethoxy, hydroxycarbonyl, methoxycarbonyl, ethoxycarbonyl and amino,
      and
      in which oxadiazolonyl, oxadiazolthionyl, phenyl, oxazolyl, thiazolyl, pyrazolyl, triazolyl, oxadiazolyl, thiadiazolyl, pyridyl, pyrimidinyl and pyrazinyl for their part may be substituted by 1 or 2 substituents independently of one another selected from the group consisting of fluorine, chlorine, cyano, difluoromethyl, trifluoromethyl, methyl, ethyl, isopropyl, 2,2,2-trifluoroethyl, 1,1,2,2,2-pentafluoroethyl, cyclopropyl, cyclobutyl, cyclopropylmethyl, cyclobutylmethyl, hydroxy, methoxy and ethoxy,
         and the salts, solvates and solvates of the salts thereof.

Preference is also given in the context of the present invention to compounds of the formula (I) in which

### A is N,

L is a #-CR^{4A}R^{4B}—(CR^{5A}R^{5B})ₚ-## group
where
# is the attachment site to the carbonyl group,
## is the point of attachment to the triazine ring,
p is a number 0,
R^{4A} is hydrogen, fluorine, methyl, ethyl, hydroxy or amino,
R^{4B} is a group of the formula -Q-R⁸,
   in which
   Q is a bond,
   R⁸ is —(C═O)ᵣ—NR⁹R¹⁰, —C(═S)—NR⁹R¹⁰, oxadiazolonyl, oxadiazolthionyl, phenyl, oxazolyl, thiazolyl, pyrazolyl, triazolyl, oxadiazolyl, thiadiazolyl, pyridyl, pyrimidinyl or pyrazinyl,
      in which
      r is the number 0,
      R⁹ and R¹⁰ are each independently of one another hydrogen, methyl, ethyl, isopropyl, cyclopropyl, cyclobutyl, cyclopentyl, oxetanyl, azetidinyl, tetrahydropyranyl, pyrrolidinyl, tetrahydropyranyl, piperidinyl, piperazinyl, morpholinyl, phenyl, pyrazolyl or pyridyl,
         in which methyl, ethyl and isopropyl for their part may be substituted by 1 or 2 substituents independently of one another selected from the group consisting of fluorine, difluoromethyl, trifluoromethyl, cyclopropyl, cyclobutyl, cyclopentyl, hydroxy, difluoromethoxy, trifluoromethoxy, methoxy, ethoxy, hydroxycarbonyl, methoxycarbonyl, ethoxycarbonyl and amino,
      and
      in which oxadiazolonyl, oxadiazolthionyl, phenyl, oxazolyl, thiazolyl, pyrazolyl, triazolyl, oxadiazolyl, thiadiazolyl, pyridyl, pyrimidinyl and pyrazinyl for their part may be substituted by 1 or 2 substituents independently of one another selected from the group consisting of fluorine, chlorine, cyano, difluoromethyl, trifluoromethyl, methyl, ethyl, isopropyl, 2,2,2-trifluoroethyl, 1,1,2,2,2-pentafluoroethyl, cyclopropyl, cyclobutyl, cyclopropylmethyl, cyclobutylmethyl, hydroxy, methoxy and ethoxy,
         and the salts, solvates and solvates of the salts thereof.

The individual radical definitions specified in the particular combinations or preferred combinations of radicals are, independently of the particular combinations of the radicals specified, also replaced as desired by radical definitions of other combinations.

Particular preference is given to combinations of two or more of the preferred ranges mentioned above.

The invention further provides a process for preparing the compounds of the formula (I) according to the invention, characterized in that
[A] a compound of the formula (II)

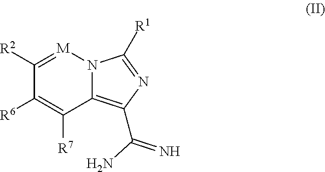

in which M, R¹, R², R⁶ and R⁷ each have the meanings given above,
are cyclized in an inert solvent in the presence of a suitable base with a compound of the formula (III)

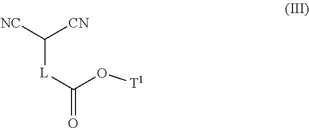

in which L has the meaning given above and
T¹ is (C₁-C₄)-alkyl
to give a compound of the formula (IV)

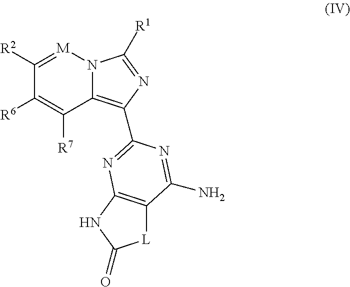

in which M, L, R¹, R², R⁶ and R⁷ each have the meanings given above,
the latter is then converted in an inert solvent using isopentyl nitrite and a halogen equivalent into a compound of the formula (I-A)

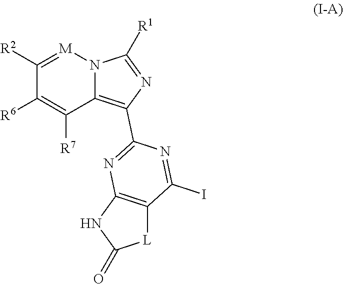

in which M, L, R¹, R², R⁶ and R⁷ each have the meanings given above,
or
[B] a compound of the formula (I-A) is reacted in an inert solvent in the presence of a suitable transition metal catalyst to give a compound of the formula (I-B)

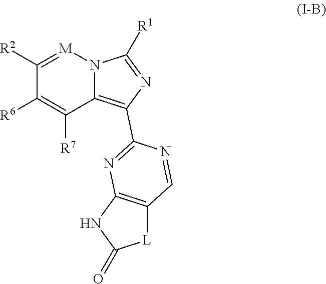

in which M, L, R¹, R², R⁶ and R⁷ each have the meanings given above,
or
[C] a compound of the formula (II) is reacted in an inert solvent in the presence of a suitable base with a compound of the formula (V)

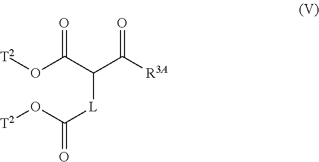

in which L has the meaning given above,
R^{3A} is hydrogen, halogen, (C₁-C₄)-alkyl or hydroxy and
T² is (C₁-C₄)-alkyl,
to give a compound of the formula (VI)

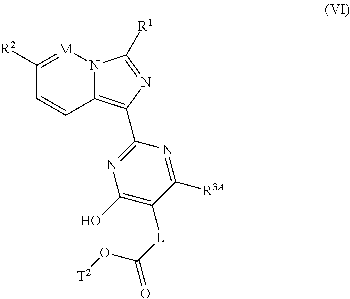

in which M, L, R¹, R², R^{3A}, R⁶, R⁷ and T² each have the meanings given above,
the latter is then converted with phosphoryl chloride into a compound of the formula (VII)

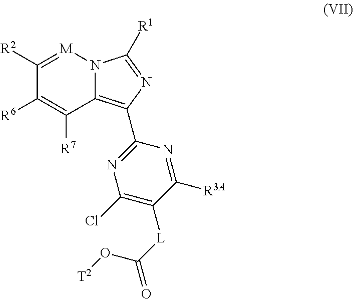

in which M, L, R¹, R², R^{3A}, R⁶, R⁷ and T² each have the meanings given above,
the latter is subsequently converted in an inert solvent into a corresponding azide compound and this is reduced directly to give a compound of the formula (VIII)

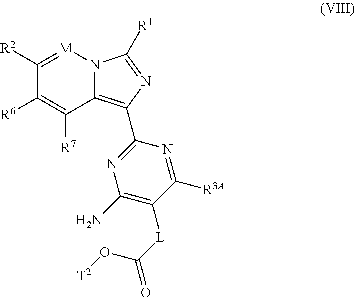

in which M, L, R¹, R², R^{3A}, R⁶, R⁷ and T² each have the meanings given above,
and the latter is then reacted in an inert solvent, in the presence of a suitable base to give a compound of the formula (I-C)

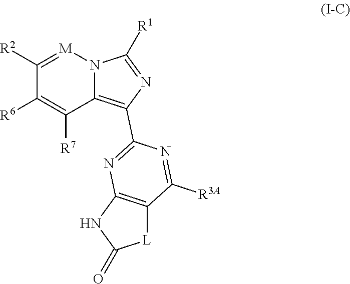

in which M, L, R¹, R² and R^{3A}, R⁶, R⁷ each have the meanings given above,
or
[D] a compound of the formula (II) is reacted in an inert solvent in the presence of a suitable base with hydrazine hydrate to give a compound of the formula (IX)

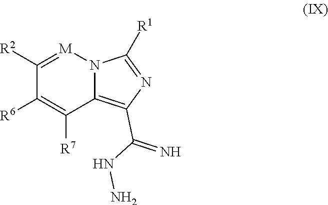

in which M, R¹, R², R⁶ and R⁷ each have the meanings given above,
the latter is then reacted in an inert solvent with a compound of the formula (X)

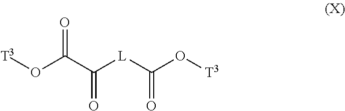

in which L has the meaning given above and
T³ is (C₁-C₄)-alkyl
to give a compound of the formula (XI)

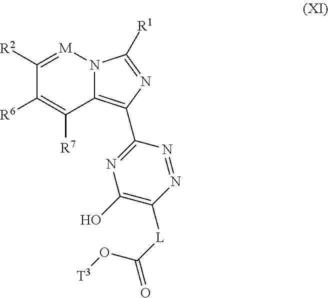

in which M, L, R¹, R², R⁶, R⁷ and T³ each have the meanings given above,
the latter is then converted into a compound of the formula (XII)

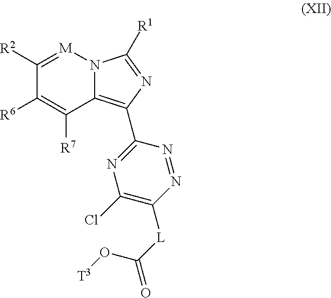

in which M, L, R¹, R², R⁶, R⁷ and T³ each have the meanings given above,
and the latter is reacted directly with ammonia to give a compound of the formula (XIII)

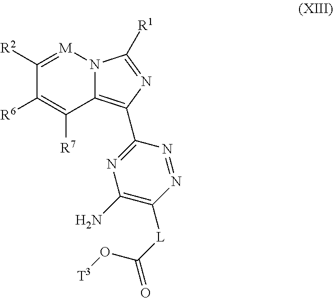

in which M, L, R¹, R², R⁶, R⁷ and T³ each have the meanings given above,
and finally cyclized in an inert solvent, optionally in the presence of a suitable base, to give a compound of the formula (I-D)

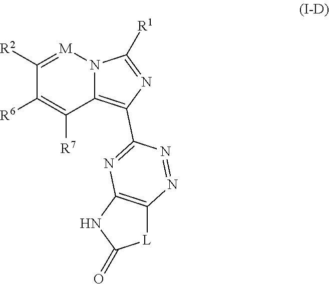

in which M, L, R¹, R², R⁶ and R⁷ each have the meanings given above,
   and the resulting compounds of the formulae (I-A), (I-B), (I-C) and (I-D) are, where appropriate, converted with the appropriate (i) solvents and/or (ii) acids or bases into their solvates, salts and/or solvates of the salts.

The compounds of the formulae (I-A), (I-B), (I-C) and (I-D) together form the group of compounds according to the invention of the formula (I).

Inert solvents for the process step (II)+(III)→(IV) are, for example, alcohols such as methanol, ethanol, n-propanol, isopropanol, n-butanol or tert-butanol, ethers such as diethyl ether, dioxane, dimethoxyethane, tetrahydrofuran, glycol dimethyl ether or diethylene glycol dimethyl ether, hydrocarbons such as benzene, xylene, toluene, hexane, cyclohexane or mineral oil fractions, or other solvents such as dimethylformamide (DMF), dimethyl sulfoxide (DMSO), N,N′-dimethylpropyleneurea (DMPU), N-methylpyrrolidone (NMP), pyridine, acetonitrile, sulfolane or else water. It is also possible to use mixtures of the solvents mentioned. Preference is given to tert-butanol or methanol.

Suitable bases for the process step (II)+(III)→(IV) are alkali metal hydroxides such as, for example, lithium hydroxide, sodium hydroxide or potassium hydroxide, alkali metal carbonates such as lithium carbonate, sodium carbonate, potassium carbonate or cesium carbonate, alkali metal bicarbonates such as sodium bicarbonate or potassium bicarbonate, alkali metal alkoxides such as sodium methoxide or potassium methoxide, sodium ethoxide or potassium ethoxide or potassium tert-butoxide, or organic amines such as triethylamine, diisopropylethylamine, pyridine, 1,8-diazabicyclo[5.4.0]undec-7-ene (DBU) or 1,5-diazabicyclo[4.3.0]non-5-ene (DBN). Preference is given to potassium tert-butoxide or sodium methoxide.

The reaction (II)+(III)→(IV) is generally carried out in a temperature range of from +20° C. to +150° C., preferably at from +75° C. to +100° C., optionally in a microwave. The reaction can be carried out at atmospheric, elevated or reduced pressure (for example from 0.5 to 5 bar). The reaction is generally carried out at atmospheric pressure.

Process step (IV)→(I-A) is carried out with or without solvent. Suitable solvents are all organic solvents which are inert under the reaction conditions. A preferred solvent is dimethoxyethane.

The reaction (IV)→(I-A) is generally carried out in a temperature range from +20° C. to +100° C., preferably in the range from +50° C. to +100° C., optionally in a microwave. The conversion can be performed at atmospheric, elevated or reduced pressure (for example in the range from 0.5 to 5 bar). The reaction is generally carried out at atmospheric pressure.

Suitable halogen sources in the reaction (IV)→(I-A) are, for example, diiodomethane, a mixture of cesium iodide, iodine and copper(I) iodide or copper(II) bromide.

Process step (IV)→(I-A), in the case of diiodomethane as the halogen source, is carried out with a molar ratio of 10 to 30 mol of isopentyl nitrite and 10 to 30 mol of the iodine equivalent based on 1 mol of the compound of the formula (IV).

Inert solvents for the process step (I-A)→(I-B) are alcohols such as methanol, ethanol, n-propanol, isopropanol, n-butanol, tert-butanol or 1,2-ethanediol, ethers such as diethyl ether, dioxane, tetrahydrofuran, glycol dimethyl ether or diethylene glycol dimethyl ether, or other solvents such as dimethylformamide (DMF), N,N′-dimethylpropyleneurea (DMPU), N-methylpyrrolidone (NMP), pyridine or else water. It is also possible to use mixtures of the solvents mentioned. Preference is given to DMF.

The reduction (I-A)→(I-B) is carried out with hydrogen in conjunction with transition metal catalysts, for example palladium (10% on activated carbon), Raney nickel or palladium hydroxide.

The reaction (I-A)→(I-B) is generally carried out in a temperature range from +20° C. to +50° C. The conversion can be carried out at atmospheric or elevated pressure (for example in the range from 0.5 to 5 bar). The reaction is generally carried out at atmospheric pressure.

Inert solvents for the process step (II)+(V)→(VI) are, for example, alcohols such as methanol, ethanol, n-propanol, isopropanol, n-butanol or tert-butanol, ethers such as diethyl ether, dioxane, dimethoxyethane, tetrahydrofuran, glycol dimethyl ether or diethylene glycol dimethyl ether, hydrocarbons such as benzene, xylene, toluene, hexane, cyclohexane or mineral oil fractions, or other solvents such as dimethylformamide (DMF), dimethyl sulfoxide (DMSO), N,N′-dimethylpropyleneurea (DMPU), N-methylpyrrolidone (NMP), pyridine or acetonitrile. It is also possible to use mixtures of the solvents mentioned. Preference is given to methanol or ethanol. Suitable bases for the process step (II)+(V)→(VI) are alkali metal hydroxides such as, for example, lithium hydroxide, sodium hydroxide or potassium hydroxide, alkali metal carbonates such as lithium carbonate, sodium carbonate, potassium carbonate or cesium carbonate, alkali metal bicarbonates such as sodium bicarbonate or potassium bicarbonate, alkali metal alkoxides such as sodium methoxide or potassium methoxide, sodium ethoxide or potassium ethoxide or potassium tert-butoxide, or organic amines such as triethylamine, diisopropylethylamine, pyridine, 1,8-diazabicyclo[5.4.0]undec-7-ene (DBU) or 1,5-diazabicyclo[4.3.0]non-5-ene (DBN). Preference is given to sodium methoxide or sodium ethoxide.

The reaction (II)+(V)→(VI) is effected generally within a temperature range from +50° C. to +120° C., preferably from +50° C. to +100° C., optionally in a microwave. The conversion can be carried out at atmospheric or elevated pressure (for example in the range from 0.5 to 5 bar). The reaction is generally carried out at atmospheric pressure.

The conversions (VI)→(VII) and (XI)→(XII) can be carried out in a solvent which is inert under the reaction conditions or without solvent. A preferred solvent is sulfolane.

The reactions (VI)→(VII) and (XI)→(XII) are generally carried out in a temperature range from +70° C. to +150° C., preferably from +80° C. to +130° C., optionally in a microwave. The conversion can be carried out at atmospheric or elevated pressure (for example in the range from 0.5 to 5 bar). The reaction is generally carried out at atmospheric pressure.

Especially preferably, the conversion (XI)→(XII) is carried out without solvent in a temperature range from 0° C. to +50° C. at atmospheric pressure.

Process step (VII)→(VIII) is carried out by reaction with sodium azide with intermediate formation of the azide derivatives which are directly reduced further to give the corresponding amines. Inert solvents for the azide formation are, for example, ethers such as diethyl ether, dioxane, dimethoxyethane, tetrahydrofuran, glycol dimethyl ether or diethylene glycol dimethyl ether, hydrocarbons such as benzene, xylene, toluene, hexane, cyclohexane or mineral oil fractions, or other solvents such as dimethylformamide (DMF), dimethyl sulfoxide (DMSO), N,N′-dimethylpropyleneurea (DMPU), N-methylpyrrolidone (NMP), pyridine, acetonitrile or sulfolane. It is also possible to use mixtures of the solvents mentioned. Preference is given to DMF.

The azide formation is generally carried out in a temperature range from +50° C. to +100° C., preferably from +60° C. to +80° C., at atmospheric pressure.

The reduction is carried out in an inert solvent such as, for example, alcohols such as methanol, ethanol, n-propanol, isopropanol, n-butanol, tert-butanol or 1,2-ethanediol, ethers such as diethyl ether, dioxane, tetrahydrofuran, glycol dimethyl ether or diethylene glycol dimethyl ether, or other solvents such as dimethylformamide (DMF), dimethyl sulfoxide (DMSO), N,N′-dimethylpropyleneurea (DMPU), N-methylpyrrolidone (NMP), pyridine, acetonitrile or else water. It is also possible to use mixtures of the solvents mentioned. Preference is given to DMF.

The reduction is carried out at from +10° C. to +30° C. using hydrogen in combination with transition metal catalysts such as, for example, palladium (10% on activated carbon), platinum dioxide or palladium hydroxide, or without hydrogen using tin(II) chloride and hydrochloric acid.

Alternatively, the reaction (VII)→(VIII) can also be carried out in one step analogously to process step (XII)→(XIII).

The cyclizations (VIII)→(I-C) and (XIII)→(I-D) are carried out in a solvent which is inert under the reaction conditions, for example alcohols such as methanol, ethanol, n-propanol, isopropanol, n-butanol or tert-butanol, ethers such as diethyl ether, dioxane, dimethoxyethane, tetrahydrofuran (THF), glycol dimethyl ether or diethylene glycol dimethyl ether, hydrocarbons such as benzene, xylene, toluene, hexane, cyclohexane or mineral oil fractions, or other solvents such as dimethylformamide (DMF), dimethyl sulfoxide (DMSO), N,N′-dimethylpropyleneurea (DMPU), N-methylpyrrolidone (NMP), pyridine, acetonitrile or sulfolane. It is also possible to use mixtures of the solvents mentioned. Preference is given to THF.

Suitable bases for the process steps (VIII)→(I-C) and (XIII)→(I-D) are alkali metal hydroxides such as, for example, lithium hydroxide, sodium hydroxide or potassium hydroxide, alkali metal carbonates such as lithium carbonate, sodium carbonate, potassium carbonate or caesium carbonate, alkali metal bicarbonates such as sodium bicarbonate or potassium bicarbonate, alkali metal alkoxides such as sodium methoxide or potassium methoxide, sodium ethoxide or potassium ethoxide or potassium tert-butoxide, or organic amines such as triethylamine, diisopropylethylamine, pyridine, 1,8-diazabicyclo[5.4.0]undec-7-ene (DBU) or 1,5-diazabicyclo[4.3.0]non-5-ene (DBN). Preference is given to potassium tert-butoxide.

The reactions (VIII)→(I-C) and (XIII)→(I-D) are generally carried out in a temperature range from 0° C. to +50° C., preferably from +10° C. to +30° C., optionally in a microwave. The conversion can be carried out at atmospheric or elevated pressure (for example in the range from 0.5 to 5 bar). The reaction is generally carried out at atmospheric pressure.

Preferably, the cyclization to (I-C) or (I-D) occurs directly during the reduction of the azide to the corresponding amine (VIII) or during the reaction (XII)→(XIII) without addition of further reagents.

In an alternative procedure for processes [C] and [D], the conversions (VII)→(VIII)→(I-C) or (IX)+(X)→(XI)→(XII)→(XIII)→(I-D) are carried out without isolation of the intermediates.

Inert solvents for the process step (II)→(IX) are, for example, alcohols such as methanol, ethanol, n-propanol, isopropanol, n-butanol or tert-butanol, ethers such as diethyl ether, dioxane, dimethoxyethane, tetrahydrofuran, glycol dimethyl ether or diethylene glycol dimethyl ether, hydrocarbons such as benzene, xylene, toluene, hexane, cyclohexane or mineral oil fractions, or other solvents such as dimethylformamide (DMF), dimethyl sulfoxide (DMSO), N,N′-dimethylpropyleneurea (DMPU), N-methylpyrrolidone (NMP), pyridine or acetonitrile. It is also possible to use mixtures of the solvents mentioned. Preference is given to ethanol.

Suitable bases for the process step (II)→(IX) are alkali metal hydroxides such as, for example, lithium hydroxide, sodium hydroxide or potassium hydroxide, alkali metal carbonates such as lithium carbonate, sodium carbonate, potassium carbonate or cesium carbonate, alkali metal bicarbonates such as sodium bicarbonate or potassium bicarbonate, alkali metal alkoxides such as sodium methoxide or potassium methoxide, sodium ethoxide or potassium ethoxide or potassium tert-butoxide, or organic amines such as triethylamine, diisopropylethylamine, pyridine, 1,8-diazabicyclo[5.4.0]undec-7-ene (DBU) or 1,5-diazabicyclo[4.3.0]non-5-ene (DBN). Preference is given to triethylamine.

The reaction (II)→(IX) is generally carried out in a temperature range from 0° C. to +60° C., preferably from +10° C. to +30° C. The conversion can be performed at atmospheric or elevated pressure (for example in the range from 0.5 to 5 bar). The reaction is generally carried out at atmospheric pressure.

Inert solvents for the process step (IX)+(X)→(XI) are, for example, alcohols such as methanol, ethanol, n-propanol, isopropanol, n-butanol or tert-butanol, ethers such as diethyl ether, dioxane, dimethoxyethane, tetrahydrofuran, glycol dimethyl ether or diethylene glycol dimethyl ether, hydrocarbons such as benzene, xylene, toluene, hexane, cyclohexane or mineral oil fractions, or other solvents such as dimethylformamide (DMF), dimethyl sulfoxide (DMSO), N,N′-dimethylpropyleneurea (DMPU), N-methylpyrrolidone (NMP), pyridine or acetonitrile. It is also possible to use mixtures of the solvents mentioned. Preference is given to methanol or ethanol.

The reaction (IX)+(X)→(XI) is generally carried out in a temperature range from +50° C. to +120° C., preferably from +50° C. to +100° C., optionally in a microwave. The conversion can be carried out at atmospheric or elevated pressure (for example in the range from 0.5 to 5 bar). The reaction is generally carried out at atmospheric pressure.

The conversion (XI)→(XII) can be carried out in a solvent which is inert under the reaction conditions, or in the absence of a solvent. The preferred solvent is sulfolane.

The reaction (XI)→(XII) is generally carried out in a temperature range from +70° C. to +150° C., preferably from +80° C. to +130° C., optionally in a microwave. The conversion can be carried out at atmospheric or elevated pressure (for example in the range from 0.5 to 5 bar). The reaction is generally carried out at atmospheric pressure.

Especially preferably, the conversion (XI)→(XII) is carried out without solvent in a temperature range from 0° C. to +50° C. at atmospheric pressure.

Process step (XII)→(XIII) is carried out in a solvent which is inert under the reaction conditions. Suitable solvents are, for example, ethers such as diethyl ether, dioxane, tetrahydrofuran, glycol dimethyl ether or diethylene glycol dimethyl ether, or other solvents such as dimethylformamide (DMF), dimethyl sulfoxide (DMSO), N,N′-dimethylpropyleneurea (DMPU), N-methylpyrrolidone (NMP), pyridine, acetonitrile or else water. It is also possible to use mixtures of the solvents mentioned. Preference is given to acetonitrile.

The reaction (XII)→(XIII) is generally carried out in a temperature range from +20° C. to +100° C., preferably from +40° C. to +70° C., optionally in a microwave. The conversion can be carried out at atmospheric or elevated pressure (for example in the range from 0.5 to 5 bar). The reaction is generally carried out at atmospheric pressure.

The cyclization (XIII)→(I-D) is carried out in a solvent which is inert under the reaction conditions, for example alcohols such as methanol, ethanol, n-propanol, isopropanol, n-butanol or tert-butanol, ethers such as diethyl ether, dioxane, dimethoxyethane, tetrahydrofuran (THF), glycol dimethyl ether or diethylene glycol dimethyl ether, hydrocarbons such as benzene, xylene, toluene, hexane, cyclohexane or mineral oil fractions, or other solvents such as dimethylformamide (DMF), dimethyl sulfoxide (DMSO), N,N′-dimethylpropyleneurea (DMPU), N-methylpyrrolidone (NMP), pyridine, acetonitrile or sulfolane. It is also possible to use mixtures of the solvents mentioned. Preference is given to THF.

Suitable bases for the process step (XIII)→(I-D) are alkali metal hydroxides such as, for example, lithium hydroxide, sodium hydroxide or potassium hydroxide, alkali metal carbonates such as lithium carbonate, sodium carbonate, potassium carbonate or cesium carbonate, alkali metal bicarbonates such as sodium bicarbonate or potassium bicarbonate, alkali metal alkoxides such as sodium methoxide or potassium methoxide, sodium ethoxide or potassium ethoxide or potassium tert-butoxide, or organic amines such as triethylamine, diisopropylethylamine, pyridine, 1,8-diazabicyclo[5.4.0]undec-7-ene (DBU) or 1,5-diazabicyclo[4.3.0]non-5-ene (DBN). Preference is given to potassium tert-butoxide.

The reaction (XIII)→(I-D) is generally carried out in a temperature range from 0° C. to +50° C., preferably from +10° C. to +30° C., optionally in a microwave. The conversion can be carried out at atmospheric or elevated pressure (for example in the range from 0.5 to 5 bar). The reaction is generally carried out at atmospheric pressure.

The reactions (XI)→(XII)→(XIII)→(I-D) are preferably carried out simultaneously in a one-pot reaction without isolation of the intermediates.

The preparation processes described can be illustrated by way of example by the following synthesis schemes (Schemes 1 to 3):

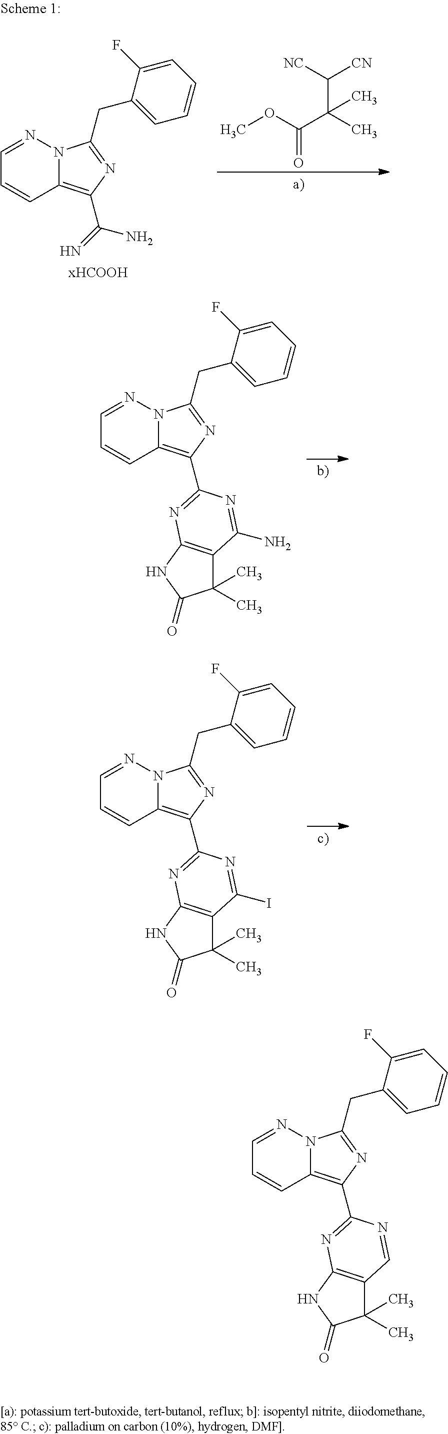 [SMILES: N=C(N)c1nc(Cc2ccccc2F)n2ncccc12 ; COC(=O)C(C)(C)C(C#N)C#N ; CC1(C)C(=O)Nc2nc(-c3nc(Cc4ccccc4F)n4ncccc34)nc(N)c21 ; CC1(C)C(=O)Nc2nc(-c3nc(Cc4ccccc4F)n4ncccc34)nc(I)c21 ; CC1(C)C(=O)Nc2nc(-c3nc(Cc4ccccc4F)n4ncccc34)ncc21]

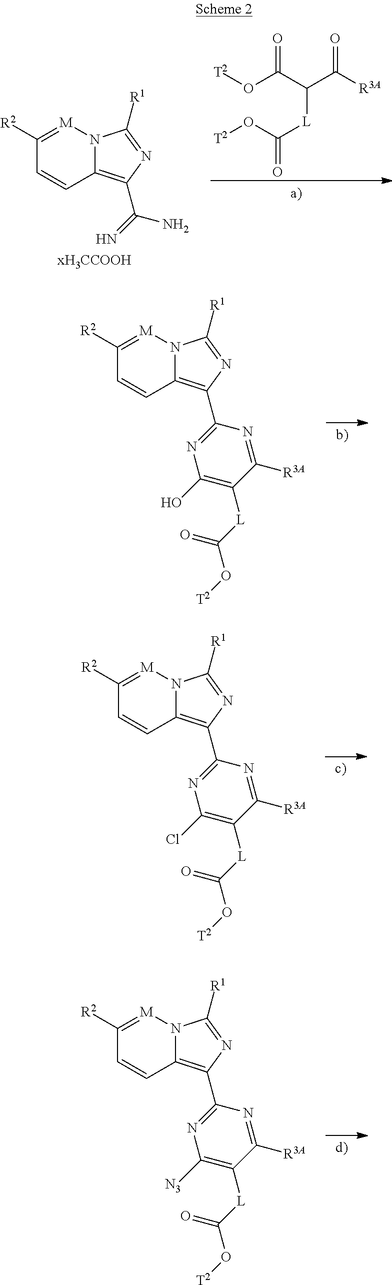

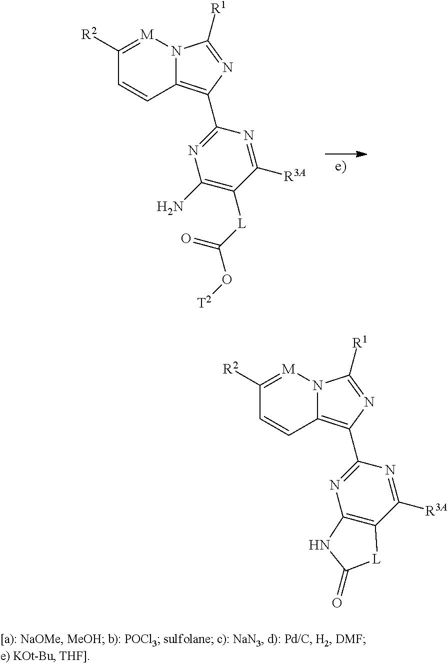

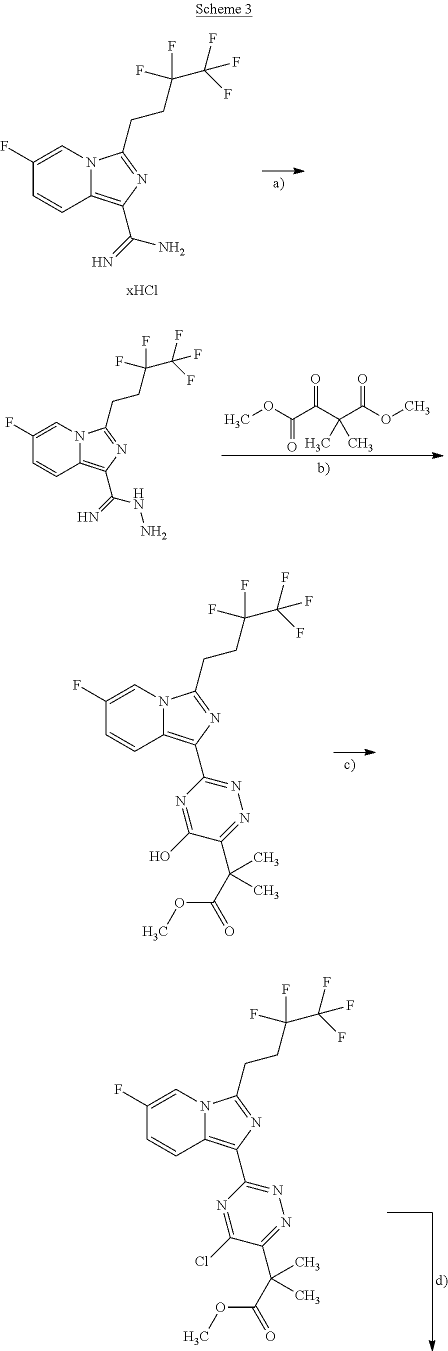

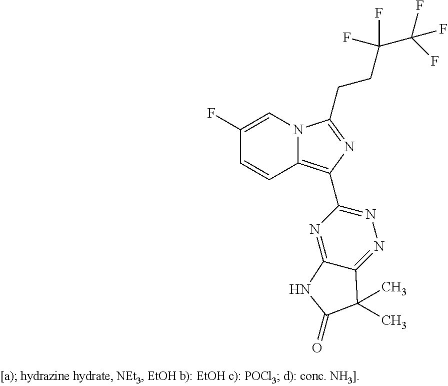 [SMILES: CC1(C)C(=O)Nc2nc(-c3nc(CCC(F)(F)C(F)(F)F)n4cc(F)ccc34)nnc21]

The compounds of the formulae (III), (V) and (X) are commercially available, known from the literature or can be prepared in analogy to literature processes.

The compounds of the formula (II) are known from the literature (see, for example, WO 2010/065275) or can be prepared by converting a compound of the formula (XIV)

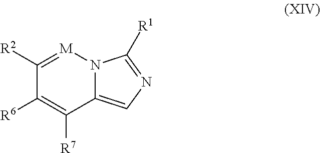

in which M, R¹, R², R⁶ and R⁷ each have the meanings given above,
in an inert solvent in the presence of a suitable halogenating agent into a compound of the formula (XV)

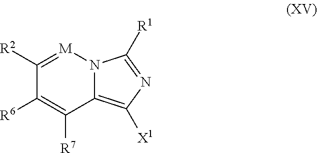

in which M, R¹, R², R⁶ and R⁷ each have the meanings given above and
X¹ is halogen, in particular bromine or iodine,
then reacting the latter in an inert solvent to give a compound of the formula (XVI)

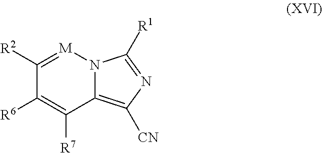

in which M, R¹, R², R⁶ and R⁷ each have the meanings given above,
and subsequently converting the latter in an inert solvent into a compound of the formula (II)

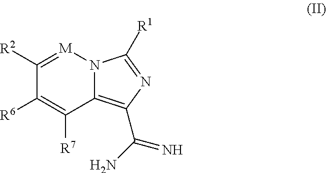

in which M, R¹, R², R⁶ and R⁷ each have the meanings given above.

Suitable inert solvents for the halogenation (XIV)→(XV) are, for example, ethers such as diethyl ether, dioxane, tetrahydrofuran, glycol dimethyl ether or diethylene glycol dimethyl ether, halogenated hydrocarbons such as dichloromethane, trichloromethane, carbon tetrachloride, trichloroethylene or chlorobenzene, or other solvents such as dimethylformamide (DMF), dimethyl sulfoxide (DMSO), N,N′-dimethylpropyleneurea (DMPU), N-methylpyrrolidone (NMP), pyridine or acetonitrile. It is also possible to use mixtures of the solvents mentioned. Preference is given to dichloromethane.

A suitable brominating agent for process step (XIV)→(XV) is elemental bromine with acetic acid, 1,3-dibromo-5,5-dimethylhydantoin and also, in particular, N-bromosuccinimide (NBS).

A particularly suitable iodinating agent for process step (XIV)→(XV) is N-iodosuccinimide (NIS).

The halogenation (XIV)→(XV) is generally carried out in a temperature range from −10° C. to +50° C., preferably from 0° C. to +30° C. The conversion can be carried out at atmospheric, elevated or reduced pressure (for example in the range from 0.5 to 5 bar). The reaction is generally carried out at atmospheric pressure.

Inert solvents for the process step (XV)→(XVI) are, for example, ethers such as diethyl ether, dioxane, tetrahydrofuran, glycol dimethyl ether or diethylene glycol dimethyl ether, hydrocarbons such as benzene, xylene, toluene, hexane, cyclohexane or mineral oil fractions, or other solvents such as dimethylformamide (DMF), dimethyl sulfoxide (DMSO), N,N′-dimethylpropyleneurea (DMPU), N-methylpyrrolidone (NMP), pyridine or acetonitrile. It is also possible to use mixtures of the solvents mentioned. Preference is given to DMSO.

The reaction (XV)→(XVI) is generally carried out in a temperature range of from +20° C. to +180° C., preferably at from +100° C. to +160° C., optionally in a microwave. The reaction can be carried out at atmospheric, elevated or reduced pressure (for example from 0.5 to 5 bar). The reaction is generally carried out at atmospheric pressure.

The reaction (XVI)→(II) is carried out using methods known to the person skilled in the art in a two-step process initially with formation of the imino ester using sodium methoxide in methanol at from 0° C. to +40° C. and subsequent nucleophilic addition of an ammonia equivalent such as, for example, ammonia or ammonium chloride in acetic acid with formation of the amidine (VII) at from +50 to +150° C.

The preparation process described can be illustrated by way of example by the following synthesis scheme (Scheme 4):

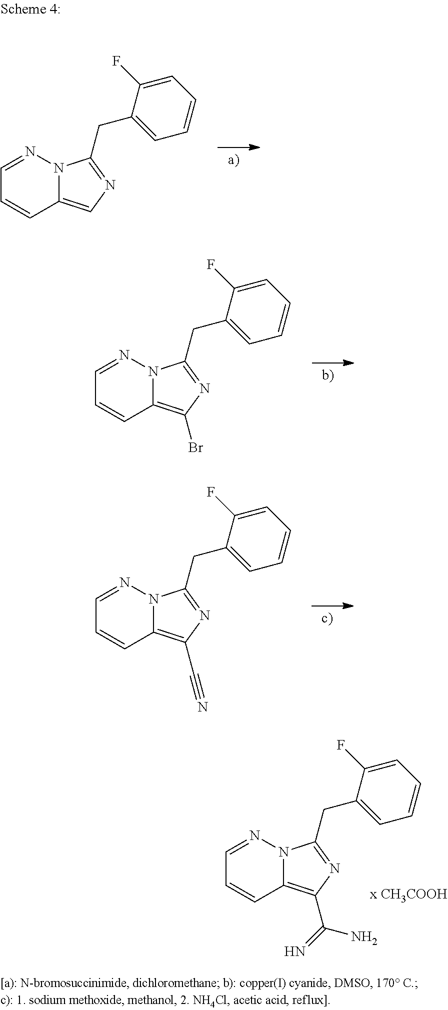 [SMILES: Fc1ccccc1Cc1ncc2cccnn12 ; Fc1ccccc1Cc1nc(Br)c2cccnn12 ; N#Cc1nc(Cc2ccccc2F)n2ncccc12 ; N=C(N)c1nc(Cc2ccccc2F)n2ncccc12]

The compounds of the formula (XIV) can be prepared by cyclizing a compound of the formula (XVII)

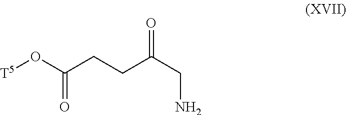

in which
T⁵ is (C₁-C₄)-alkyl
in an inert solvent in the presence of a suitable base with hydrazine hydrate to give the compound of the formula (XVIII)

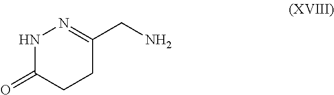

then reacting the latter in an inert solvent, in the presence of a suitable base, with a compound of the formula (XIX)

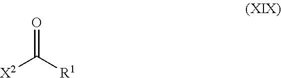

in which R¹ has the meaning given above and
X² is halogen, in particular chlorine or bromine,
to give a compound of the formula (XX)

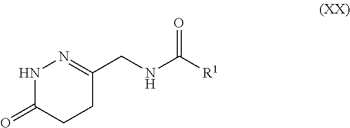

in which R¹ has the meaning given above,
then oxidizing the latter to a compound of the formula (XXI)

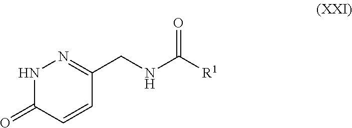

in which R¹ has the meaning given above,
furthermore cyclizing the latter in the absence of a solvent or in an inert solvent with phosphorus oxychloride to give a compound of the formula (XXII)

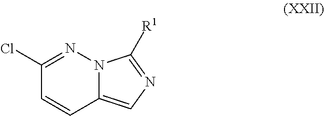

in which R¹ has the meaning given above
and finally hydrogenating in an inert solvent in the presence of a suitable base.

The compounds of the formulae (XVII) and (XVIII) are commercially available, known from the literature or can be prepared in analogy to literature processes.

Inert solvents for the process step (XVII)→(XVIII) are alcohols such as methanol, ethanol, n-propanol, isopropanol, n-butanol or tert-butanol, ethers such as diethyl ether, dioxane, tetrahydrofuran, glycol dimethyl ether or diethylene glycol dimethyl ether, hydrocarbons such as benzene, xylene, toluene, hexane, cyclohexane or mineral oil fractions, or other solvents such as dimethylformamide (DMF), dimethyl sulfoxide (DMSO), N,N′-dimethylpropyleneurea (DMPU), N-methylpyrrolidone (NMP), pyridine, acetonitrile or else water. It is also possible to use mixtures of the solvents mentioned. Preference is given to ethanol.

Suitable bases for the process step (XVII)→(XVIII) are alkali metal hydroxides such as, for example, lithium hydroxide, sodium hydroxide or potassium hydroxide, alkali metal carbonates such as lithium carbonate, sodium carbonate, potassium carbonate or cesium carbonate, alkali metal bicarbonates such as sodium bicarbonate or potassium bicarbonate, alkali metal alkoxides such as sodium methoxide or potassium methoxide, sodium ethoxide or potassium ethoxide or potassium tert-butoxide, or organic amines such as triethylamine, diisopropylethylamine, pyridine, 1,8-diazabicyclo[5.4.0]undec-7-ene (DBU) or 1,5-diazabicyclo[4.3.0]non-5-ene (DBN). Preference is given to triethylamine.

The reaction (XVII)→(XVIII) is generally carried out in a temperature range of from +20° C. to +150° C., preferably at from +80° C. to +120° C., optionally in a microwave. The reaction can be carried out at atmospheric, elevated or reduced pressure (for example from 0.5 to 5 bar). The reaction is generally carried out at atmospheric pressure.

Inert solvents for the process step (XVIII)+(XIX)→(XX) are ethers such as diethyl ether, dioxane, tetrahydrofuran, glycol dimethyl ether or diethylene glycol dimethyl ether, hydrocarbons such as benzene, xylene, toluene, hexane, cyclohexane or mineral oil fractions, halogenated hydrocarbons such as dichloromethane, trichloromethane, carbon tetrachloride, trichloroethylene or chlorobenzene, or other solvents such as dimethylformamide (DMF), dimethyl sulfoxide (DMSO), N,N′-dimethylpropyleneurea (DMPU), N-methylpyrrolidone (NMP), pyridine or acetonitrile. It is also possible to use mixtures of the solvents mentioned. Preference is given to acetonitrile.

Suitable bases for the process step (XVIII)+(XIX)→(XX) are alkali metal carbonates such as lithium carbonate, sodium carbonate, potassium carbonate or cesium carbonate, or organic amines such as triethylamine, diisopropylethylamine, pyridine, 1,8-diazabicyclo[5.4.0]undec-7-ene (DBU) or 1,5-diazabicyclo[4.3.0]non-5-ene (DBN). Preference is given to triethylamine

The reaction (XVIII)+(XIX)→(XX) is generally carried out in a temperature range from −20° C. to +40° C., preferably at from 0° C. to +20° C. The reaction can be carried out at atmospheric, elevated or reduced pressure (for example from 0.5 to 5 bar). The reaction is generally carried out at atmospheric pressure.

The oxidation (XX)→(XXI) is preferably carried out in an organic acid such as, for example, formic acid or acetic acid, in the presence of elemental bromine at a temperature of from +40° C. to +100° C.

The cyclization (XXI)→(XXII) is carried out in the absence of a solvent or in a solvent which is inert under the reaction conditions. Suitable solvents are, for example, hydrocarbons such as benzene, xylene, toluene, hexane, cyclohexane or mineral oil fractions, or other solvents such as dimethylformamide (DMF), dimethyl sulfoxide (DMSO), N,N′-dimethylpropyleneurea (DMPU), N-methylpyrrolidone (NMP), pyridine, sulfolane or acetonitrile. Preference is given to using sulfolane.

The cyclization (XXI)→(XXII) is generally carried out in a temperature range from +50° C. to +140° C., preferably at from +80° C. to +120° C. The reaction can be carried out at atmospheric, elevated or reduced pressure (for example from 0.5 to 5 bar). The reaction is generally carried out at atmospheric pressure.

The process step (XXII)→(XIV) is carried out in a solvent which is inert under the reaction conditions, for example alcohols such as methanol, ethanol, n-propanol, isopropanol, n-butanol or tert-butanol, ethers such as diethyl ether, dioxane, tetrahydrofuran, glycol dimethyl ether or diethylene glycol dimethyl ether, hydrocarbons such as benzene, xylene, toluene, hexane, cyclohexane or mineral oil fractions, or other solvents such as ethyl acetate, dimethylformamide (DMF), dimethyl sulfoxide (DMSO), N,N′-dimethylpropyleneurea (DMPU), N-methylpyrrolidone (NMP), pyridine, sulfolane or acetonitrile. Preference is given to ethyl acetate.

Suitable bases for the process step (XXII)→(XIV) are organic amines such as triethylamine, diisopropylethylamine, pyridine, 1,8-diazabicyclo[5.4.0]undec-7-ene (DBU) or 1,5-diazabicyclo[4.3.0]non-5-ene (DBN). Preference is given to triethylamine.

The cyclization (XXII)→(XIV) is generally carried out in a temperature range from 0° C. to +60° C., preferably at from +10° C. to +30° C.

The scheme below (Scheme 5) shows, in an exemplary manner, the preparation process described above:

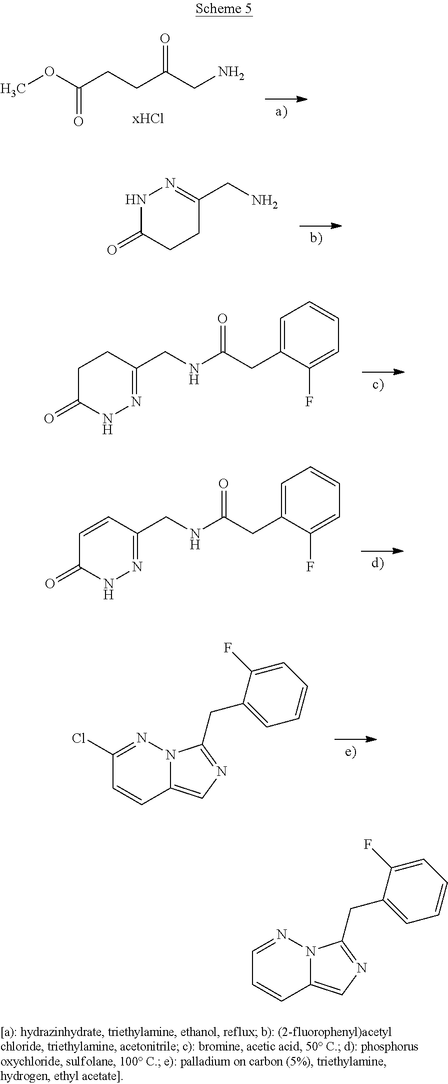

The compounds according to the invention act as potent stimulators of soluble guanylate cyclase and have an identical or improved therapeutic profile compared to compounds known from the prior art, such as, for example, with respect to their in vivo properties such as, for example, their pharmacokinetic and pharmacodynamic behavior and/or their dose-activity relationship and/or their safety profile. They are therefore suitable for the treatment and/or prophylaxis of diseases in man and animals.

The compounds according to the invention cause vasorelaxation and inhibition of platelet aggregation, and lead to a decrease in blood pressure and to a rise in coronary blood flow. These effects are mediated by direct stimulation of soluble guanylate cyclase and an intracellular rise in cGMP. In addition, the compounds according to the invention enhance the action of substances which increase the cGMP level, for example EDRF (endothelium-derived relaxing factor), NO donors, protoporphyrin IX, arachidonic acid or phenylhydrazine derivatives.

The compounds according to the invention are suitable for treatment and/or prophylaxis of cardiovascular, pulmonary, thromboembolic and fibrotic disorders.

The compounds according to the invention can therefore be used in medicaments for treatment and/or prophylaxis of cardiovascular disorders, for example hypertension, acute and chronic heart failure, coronary heart disease, stable and unstable angina pectoris, peripheral and cardiovascular disorders, arrhythmias, atrial and ventricular arrhythmias and impaired conduction, for example atrioventricular grade I-III blocks (AB block I-III), supraventricular tachyarrhythmia, atrial fibrillation, atrial flutter, ventricular fibrillation, ventricular flutter, ventricular tachyarrhythmia, Torsade de pointes tachycardia, atrial and ventricular extrasystoles, AV-junctional extrasystoles, Sick-Sinus syndrome, syncopes, AV-nodal re-entry tachycardia, Wolff-Parkinson-White syndrome, acute coronary syndrome (ACS), autoimmune cardiac disorders (pericarditis, endocarditis, valvolitis, aortitis, cardiomyopathies), shock such as cardiogenic shock, septic shock and anaphylactic shock, aneurysms, boxer cardiomyopathy (premature ventricular contraction (PVC)), for treatment and/or prophylaxis of thromboembolic disorders and ischemias such as myocardial ischemia, myocardial infarction, stroke, cardiac hypertrophy, transient and ischemic attacks, preeclampsia, inflammatory cardiovascular disorders, spasms of the coronary arteries and peripheral arteries, edema formation, for example pulmonary edema, cerebral edema, renal edema or edema caused by heart failure, impaired peripheral perfusion, reperfusion damage, arterial and venous thromboses, microalbuminuria, myocardial insufficiency, endothelial dysfunction, for prevention of restenoses, such as after thrombolysis treatments, percutaneous transluminal angioplasties (PTA), transluminal coronary angioplasties (PTCA), heart transplants and bypass operations, and micro- and macrovascular damage (vasculitis), elevated levels of fibrinogen and of low-density LDL, and elevated concentrations of plasminogen activator inhibitor 1 (PAI-1), and for treatment and/or prophylaxis of erectile dysfunction and female sexual dysfunction.

In the context of the present invention, the term “heart failure” also encompasses both acute and chronic forms of heart failure, and also more specific or related types of disease, such as acute decompensated heart failure, right heart failure, left heart failure, global failure, ischemic cardiomyopathy, dilated cardiomyopathy, hypertrophic cardiomyopathy, idiopathic cardiomyopathy, congenital heart defects, heart failure associated with heart valve defects, mitral valve stenosis, mitral valve insufficiency, aortic valve stenosis, aortic valve insufficiency, tricuspid valve stenosis, tricuspid valve insufficiency, pulmonary valve stenosis, pulmonary valve insufficiency, combined heart valve defects, myocardial inflammation (myocarditis), chronic myocarditis, acute myocarditis, viral myocarditis, diabetic heart failure, alcoholic cardiomyopathy, cardiac storage disorders, diastolic heart failure and systolic heart failure, and acute phases of worsening of existing chronic heart failure (worsening heart failure).

In addition, the compounds according to the invention can also be used for the treatment and/or prophylaxis of arteriosclerosis, impaired lipid metabolism, hypolipoproteinemias, dyslipidemias, hypertriglyceridemias, hyperlipidemias, hypercholesterolemias, abetalipoproteinemias, sitosterolemia, xanthomatosis, Tangier disease, adiposity, obesity and of combined hyperlipidemias and metabolic syndrome.

Moreover, the compounds according to the invention can be used for treatment and/or prophylaxis of primary and secondary Raynaud's phenomenon, of microcirculation disorders, claudication, peripheral and autonomic neuropathies, diabetic microangiopathies, diabetic retinopathy, diabetic ulcers at the extremities, gangrene, CREST syndrome, erythematosis, onychomycosis, rheumatic disorders, and for promotion of wound healing.

The compounds according to the invention are furthermore suitable for treating urological disorders such as, for example, benign prostate syndrome (BPS), benign prostate hyperplasia (BPH), benign prostate enlargement (BPE), bladder outlet obstruction (BOO), lower urinary tract syndromes (LUTS, including Feline Urological Syndrome (FUS)), disorders of the urogenital system including neurogenic over-active bladder (OAB) and (IC), incontinence (UI) such as, for example, mixed urinary incontinence, urge urinary incontinence, stress urinary incontinence or overflow urinary incontinence (MUI, UUI, SUI, OUI), pelvic pain, benign and malignant disorders of the organs of the male and femal urogenital system.

The compounds according to the invention are furthermore suitable for the treatment and/or prophylaxis of kidney disorders, in particular of acute and chronic renal insufficiency and acute and chronic renal failure. In the context of the present invention, the term “renal insufficiency” encompasses both acute and chronic manifestations of renal insufficiency, and also underlying or related renal disorders such as renal hypoperfusion, intradialytic hypotension, obstructive uropathy, glomerulopathies, glomerulonephritis, acute glomerulonephritis, glomerulosclerosis, tubulointerstitial diseases, nephropathic disorders such as primary and congenital kidney disease, nephritis, immunological kidney disorders such as kidney transplant rejection and immunocomplex-induced kidney disorders, nephropathy induced by toxic substances, nephropathy induced by contrast agents, diabetic and non-diabetic nephropathy, pyelonephritis, renal cysts, nephrosclerosis, hypertensive nephrosclerosis and nephrotic syndrome which can be characterized diagnostically, for example, by abnormally reduced creatinine and/or water excretion, abnormally elevated blood concentrations of urea, nitrogen, potassium and/or creatinine, altered activity of renal enzymes, for example glutamyl synthetase, altered urine osmolarity or urine volume, elevated microalbuminuria, macroalbuminuria, lesions on glomerulae and arterioles, tubular dilatation, hyperphosphatemia and/or need for dialysis. The present invention also encompasses the use of the compounds according to the invention for treatment and/or prophylaxis of sequelae of renal insufficiency, for example pulmonary edema, heart failure, uremia, anemia, electrolyte disturbances (for example hypercalemia, hyponatremia) and disturbances in bone and carbohydrate metabolism.

Furthermore, the compounds according to the invention are also suitable for treatment and/or prophylaxis of asthmatic disorders, pulmonary arterial hypertension (PAH) and other forms of pulmonary hypertension (PH) including pulmonary hypertension associated with left heart disease, HIV, sickle cell anemia, thromboembolisms (CTEPH), sarcoidosis, COPD or pulmonary fibrosis, or chronic-obstructive pulmonary disease (COPD), acute respiratory distress syndrome (ARDS), acute lung injury (ALI), alpha-1 antitrypsin deficiency (AATD), pulmonary fibrosis, pulmonary emphysema (for example pulmonary emphysema induced by cigarette smoke) and cystic fibrosis (CF).

The compounds described in the present invention are also active compounds for control of central nervous system disorders characterized by disturbances of the NO/cGMP system. More particularly, they are suitable for improving perception, concentration, learning or memory after cognitive impairments such as those occurring particularly in the event of situations/diseases/syndromes such as mild cognitive impairment, age-associated learning and memory impairments, age-associated memory losses, vascular dementia, craniocerebral trauma, stroke, dementia occurring after strokes (post-stroke dementia), post-traumatic craniocerebral trauma, general concentration impairments, concentration impairments in children having learning and memory problems, Alzheimer's disease, Lewy body dementia, dementia with degeneration of the frontal lobes including Pick's syndrome, Parkinson's disease, progressive nuclear palsy, dementia with corticobasal degeneration, amyolateral sclerosis (ALS), Huntington's disease, demyelination, multiple sclerosis, thalamic degeneration, Creutzfeld-Jacob dementia, HIV dementia, schizophrenia with dementia or Korsakoff s psychosis. They are also suitable for treatment and/or prophylaxis of central nervous system disorders such as states of anxiety, tension and depression, CNS-related sexual dysfunction and disrupted sleep, and for control of pathological disturbances of the intake of food, stimulants and addictive substances.

Furthermore, the compounds according to the invention are also suitable for regulating cerebral blood flow and are thus effective agents for control of migraine. They are also suitable for prophylaxis and control of sequelae of cerebral infarct (cerebral apoplexy) such as stroke, cerebral ischemia and craniocerebral trauma. The compounds according to the invention can likewise be employed for controlling states of pain and tinnitus.

In addition, the compounds according to the invention have antiinflammatory action and can therefore be used as antiinflammatory agents for the treatment and/or prophylaxis of sepsis (SIRS), multiple organ failure (MODS, MOF), inflammatory disorders of the kidney, chronic intestinal inflammations (IBD, Crohn's disease, UC), pancreatitis, peritonitis, rheumatoid disorders, inflammatory skin diseases and inflammatory eye diseases.

Furthermore, the compounds according to the invention can also be used for the treatment and/or prophylaxis of autoimmune diseases.

Furthermore, the compounds according to the invention are suitable for treatment and/or prophylaxis of fibrotic disorders of the internal organs, for example of the lung, of the heart, of the kidneys, of the bone marrow and especially of the liver, and also of dermatological fibroses and fibrotic disorders of the eye. In the context of the present inventions, the term “fibrotic disorders” encompasses especially the following terms: hepatic fibrosis, hepatic cirrhosis, pulmonary fibrosis, endomyocardial fibrosis, nephropathy, glomerulonephritis, interstitial renal fibrosis, fibrotic damage resulting from diabetes, myelofibrosis and similar fibrotic disorders, scleroderma, morphea, keloids, hypertrophic scarring (including after surgical interventions), naevi, diabetic retinopathy, proliferative vitreoretinopathy and disorders of the connective tissue (for example sarcoidosis).

Furthermore, the compounds according to the invention are suitable for control of postoperative scarring, for example resulting from glaucoma operations.

The compounds according to the invention can also be used cosmetically for aging and keratinized skin.

Moreover, the compounds according to the invention are suitable for the treatment and/or prophylaxis of hepatitis, neoplasms, osteoporosis, glaucoma and gastroparesis.

The present invention further provides for the use of the compounds according to the invention for the treatment and/or prophylaxis of disorders, in particular the disorders mentioned above.

The present invention further provides for the use of the compounds according to the invention for treatment and/or prophylaxis of heart failure, angina pectoris, hypertension, pulmonary hypertension, ischemia, vascular disorders, renal insufficiency, thromboembolic disorders, fibrotic disorders and arteriosclerosis.

The present invention further provides the compounds according to the invention for use in a method for treatment and/or prophylaxis of heart failure, angina pectoris, hypertension, pulmonary hypertension, ischemia, vascular disorders, kidney failure, thromboembolic disorders, fibrotic disorders and arteriosclerosis.

The present invention further provides for the use of the compounds according to the invention for production of a medicament for treatment and/or prophylaxis of disorders, especially of the aforementioned disorders.

The present invention further provides for the use of the compounds according to the invention for production of a medicament for treatment and/or prophylaxis of heart failure, angina pectoris, hypertension, pulmonary hypertension, ischemia, vascular disorders, kidney failure, thromboembolic disorders, fibrotic disorders and arteriosclerosis.

The present invention further provides a method for treatment and/or prophylaxis of disorders, in particular the disorders mentioned above, using an effective amount of at least one of the compounds according to the invention.

The present invention further provides a method for treatment and/or prophylaxis of heart failure, angina pectoris, hypertension, pulmonary hypertension, ischemia, vascular disorders, kidney failure, thromboembolic disorders, fibrotic disorders and arteriosclerosis using an effective amount of at least one of the compounds according to the invention.

The compounds according to the invention can be used alone or, if required, in combination with other active compounds. The present invention further provides medicaments comprising at least one of the compounds according to the invention and one or more further active compounds, especially for the treatment and/or prophylaxis of the aforementioned disorders. Preferred examples of suitable active compound combinations include:
organic nitrates and NO donors, for example sodium nitroprusside, nitroglycerine, isosorbide mononitrate, isosorbide dinitrate, molsidomine or SIN1, and inhaled NO;
compounds which inhibit the breakdown of cyclic guanosine monophosphate (cGMP), for example inhibitors of phosphodiesterases (PDE) 1, 2 and/or 5, especially PDE 5 inhibitors such as sildenafil, vardenafil and tadalafil;
antithrombotic agents, by way of example and with preference from the group of the platelet aggregation inhibitors, the anticoagulants or the profibrinolytic substances;
hypotensive active compounds, by way of example and with preference from the group of the calcium antagonists, angiotensin AII antagonists, ACE inhibitors, endothelin antagonists, renin inhibitors, alpha-receptor blockers, beta-receptor blockers, mineralocorticoid receptor antagonists, and the diuretics; and/or
active compounds which modify lipid metabolism, by way of example and with preference from the group of thyroid receptor agonists, cholesterol synthesis inhibitors such as, by way of example and with preference, HMG-CoA reductase inhibitors or squalene synthesis inhibitors, ACAT inhibitors, CETP inhibitors, MTP inhibitors, PPAR-alpha, PPAR-gamma and/or PPAR-delta agonists, cholesterol absorption inhibitors, lipase inhibitors, polymeric bile acid adsorbents, bile acid reabsorption inhibitors and lipoprotein (a) antagonists.

Antithrombotic agents are preferably understood to mean compounds from the group of the platelet aggregation inhibitors, the anticoagulants or the profibrinolytic substances.

In a preferred embodiment of the invention, the compounds according to the invention are administered in combination with a platelet aggregation inhibitor such as, by way of example and with preference aspirin, clopidogrel, ticlopidin or dipyridamol.

In a preferred embodiment of the invention, the compounds according to the invention are administered in combination with a thrombin inhibitor, by way of example and with preference ximelagatran, melagatran, bivalirudin or clexane.

In a preferred embodiment of the invention, the compounds according to the invention are administered in combination with a GPIIb/IIIa antagonist such as, by way of example and with preference tirofiban or abciximab.

In a preferred embodiment of the invention, the compounds according to the invention are administered in combination with a factor Xa inhibitor, by way of example and with preference rivaroxaban (BAY 59-7939), DU176b, apixaban, otamixaban, fidexaban, razaxaban, fondaparinux, idraparinux, PMD-3112, YM-150, KFA-1982, EMD-503982, MCM-17, MLN-1021, DX 9065a, DPC 906, JTV 803, SSR-126512 or SSR-128428.

In a preferred embodiment of the invention, the compounds according to the invention are administered in combination with heparin or with a low molecular weight (LMW) heparin derivative.

In a preferred embodiment of the invention, the compounds according to the invention are administered in combination with a vitamin K antagonist, by way of example and with preference coumarin.

Hypotensive agents are preferably understood to mean compounds from the group of calcium antagonists, angiotensin AII antagonists, ACE inhibitors, endothelin antagonists, renin inhibitors, alpha-receptor blockers, beta-receptor blockers, mineralocorticoid receptor antagonists, and the diuretics.

In a preferred embodiment of the invention, the compounds according to the invention are administered in combination with a calcium antagonist, by way of example and with preference nifedipine, amlodipine, verapamil or diltiazem.

In a preferred embodiment of the invention, the compounds according to the invention are administered in combination with an alpha-1-receptor blocker, by way of example and with preference prazosin.

In a preferred embodiment of the invention, the compounds according to the invention are administered in combination with a beta-receptor blocker, by way of example and with preference propranolol, atenolol, timolol, pindolol, alprenolol, oxprenolol, penbutolol, bupranolol, metipranolol, nadolol, mepindolol, carazalol, sotalol, metoprolol, betaxolol, celiprolol, bisoprolol, carteolol, esmolol, labetalol, carvedilol, adaprolol, landiolol, nebivolol, epanolol or bucindolol.

In a preferred embodiment of the invention, the compounds according to the invention are administered in combination with an angiotensin AII antagonist, by way of example and with preference losartan, candesartan, valsartan, telmisartan or embusartan.

In a preferred embodiment of the invention, the compounds according to the invention are administered in combination with an ACE inhibitor, by way of example and with preference enalapril, captopril, lisinopril, ramipril, delapril, fosinopril, quinopril, perindopril or trandopril.

In a preferred embodiment of the invention, the compounds according to the invention are administered in combination with an endothelin antagonist, by way of example and with preference bosentan, darusentan, ambrisentan or sitaxsentan.

In a preferred embodiment of the invention, the compounds according to the invention are administered in combination with a renin inhibitor, by way of example and with preference aliskiren, SPP600 or SPP800.

In a preferred embodiment of the invention, the compounds according to the invention are administered in combination with a mineralocorticoid receptor antagonist, by way of example and with preference spironolactone or eplerenone.

In a preferred embodiment of the invention, the compounds according to the invention are administered in combination with a loop diuretic, for example furosemide, torasemide, bumetanide and piretanide, with potassium-sparing diuretics, for example amiloride and triamterene, with aldosterone antagonists, for example spironolactone, potassium canrenoate and eplerenone, and also thiazide diuretics, for example hydrochlorothiazide, chlorthalidone, xipamide and indapamide.

Lipid metabolism modifiers are preferably understood to mean compounds from the group of the CETP inhibitors, thyroid receptor agonists, cholesterol synthesis inhibitors such as HMG-CoA reductase inhibitors or squalene synthesis inhibitors, the ACAT inhibitors, MTP inhibitors, PPAR-alpha, PPAR-gamma and/or PPAR-delta agonists, cholesterol absorption inhibitors, polymeric bile acid adsorbents, bile acid reabsorption inhibitors, lipase inhibitors and the lipoprotein (a) antagonists.

In a preferred embodiment of the invention, the compounds according to the invention are administered in combination with a CETP inhibitor, by way of example and with preference dalcetrapib, BAY 60-5521, anacetrapib oder CETP vaccine (CETi-1).

In a preferred embodiment of the invention, the compounds according to the invention are administered in combination with a thyroid receptor agonist, by way of example and with preference D-thyroxine, 3,5,3′-triiodothyronine (T3), CGS 23425 or axitirome (CGS 26214).

In a preferred embodiment of the invention, the compounds according to the invention are administered in combination with an HMG-CoA reductase inhibitor from the class of statins, by way of example and with preference lovastatin, simvastatin, pravastatin, fluvastatin, atorvastatin, rosuvastatin or pitavastatin.

In a preferred embodiment of the invention, the compounds according to the invention are administered in combination with a squalene synthesis inhibitor, by way of example and with preference BMS188494 or TAK475.

In a preferred embodiment of the invention, the compounds according to the invention are administered in combination with an ACAT inhibitor, by way of example and with preference avasimibe, melinamide, pactimibe, eflucimibe or SMP797.

In a preferred embodiment of the invention, the compounds according to the invention are administered in combination with an MTP inhibitor, by way of example and with preference implitapide, BMS201038, R103757 or JTT130.

In a preferred embodiment of the invention, the compounds according to the invention are administered in combination with a PPAR-gamma agonist, by way of example and with preference pioglitazone or rosiglitazone.

In a preferred embodiment of the invention, the compounds according to the invention are administered in combination with a PPAR-delta agonist, by way of example and with preference GW 501516 or BAY 685042.

In a preferred embodiment of the invention, the compounds according to the invention are administered in combination with a cholesterol absorption inhibitor, by way of example and with preference ezetimibe, tiqueside or pamaqueside.

In a preferred embodiment of the invention, the compounds according to the invention are administered in combination with a lipase inhibitor, by way of example and with preference orlistat.

In a preferred embodiment of the invention, the compounds according to the invention are administered in combination with a polymeric bile acid adsorbent, by way of example and with preference cholestyramine, colestipol, colesolvam, CholestaGel or colestimide.

In a preferred embodiment of the invention, the compounds according to the invention are administered in combination with a bile acid reabsorption inhibitor, by way of example and with preference ASBT (=IBAT) inhibitors, for example AZD7806, S8921, AK105, BARI1741, SC435 or SC635.

In a preferred embodiment of the invention, the compounds according to the invention are administered in combination with a lipoprotein (a) antagonist, by way of example and with preference gemcabene calcium (CI1027) or nicotinic acid.

The present invention further provides medicaments which comprise at least one compound according to the invention, typically together with one or more inert, nontoxic, pharmaceutically suitable auxiliaries, and for the use thereof for the aforementioned purposes.

The compounds according to the invention may act systemically and/or locally. For this purpose, they can be administered in a suitable manner, for example by the oral, parenteral, pulmonal, nasal, sublingual, lingual, buccal, rectal, dermal, transdermal, conjunctival, otic route, or as an implant or stent.

The compounds according to the invention can be administered in administration forms suitable for these administration routes.

Suitable administration forms for oral administration are those which work according to the prior art, which release the compounds according to the invention rapidly and/or in a modified manner and which contain the compounds according to the invention in crystalline and/or amorphized and/or dissolved form, for example tablets (uncoated or coated tablets, for example with gastric juice-resistant or retarded-dissolution or insoluble coatings which control the release of the compound according to the invention), tablets or films/oblates which disintegrate rapidly in the oral cavity, films/lyophilizates or capsules (for example hard or soft gelatin capsules), sugar-coated tablets, granules, pellets, powders, emulsions, suspensions, aerosols or solutions.

Parenteral administration can be accomplished with avoidance of an absorption step (for example by an intravenous, intraarterial, intracardiac, intraspinal or intralumbar route) or with inclusion of an absorption (for example by an intramuscular, subcutaneous, intracutaneous, percutaneous or intraperitoneal route). Administration forms suitable for parenteral administration include preparations for injection and infusion in the form of solutions, suspensions, emulsions, lyophilizates or sterile powders.

For the other administration routes, suitable examples are inhalable medicament forms (including powder inhalers, nebulizers), nasal drops, solutions or sprays, tablets, films/oblates or capsules for lingual, sublingual or buccal administration, suppositories, ear or eye preparations, vaginal capsules, aqueous suspensions (lotions, shaking mixtures), lipophilic suspensions, ointments, creams, transdermal therapeutic systems (e.g. patches), milk, pastes, foams, sprinkling powders, implants or stents.

Preference is given to oral or parenteral administration, especially oral administration.

The compounds according to the invention can be converted to the administration forms mentioned. This can be accomplished in a manner known per se by mixing with inert nontoxic pharmaceutically suitable auxiliaries. These auxiliaries include carriers (for example microcrystalline cellulose, lactose, mannitol), solvents (e.g. liquid polyethylene glycols), emulsifiers and dispersing or wetting agents (for example sodium dodecylsulfate, polyoxysorbitan oleate), binders (for example polyvinylpyrrolidone), synthetic and natural polymers (for example albumin), stabilizers (e.g. antioxidants, for example ascorbic acid), dyes (e.g. inorganic pigments, for example iron oxides) and flavor and/or odor correctors.

In general, it has been found to be advantageous in the case of parenteral administration to administer amounts of from about 0.001 to 1 mg/kg, preferably about 0.01 to 0.5 mg/kg, of body weight to achieve effective results. In the case of oral administration, the dose is about 0.001 to 2 mg/kg, preferably about 0.001 to 1 mg/kg, of body weight.

It may nevertheless be necessary where appropriate to deviate from the stated amounts, specifically as a function of the body weight, route of administration, individual response to the active compound, nature of the preparation and time or interval over which administration takes place. For instance, in some cases, less than the aforementioned minimum amount may be sufficient, while in other cases the upper limit mentioned must be exceeded. In the case of administration of relatively large amounts, it may be advisable to divide these into several individual doses over the course of the day.

The working examples which follow illustrate the invention. The invention is not restricted to the examples.

The percentages in the tests and examples which follow are, unless stated otherwise, percentages by weight; parts are parts by weight. Solvent ratios, dilution ratios and concentration figures for liquid/liquid solutions are each based on volume.

### A. EXAMPLES

### Abbreviations and Acronyms

aq. aqueous solution
calc. calculated
DCI direct chemical ionization (in MS)
DMF dimethylformamide
DMSO dimethyl sulfoxide
eq. equivalent(s)
ESI electrospray ionization (in MS)
Et ethyl
h hour(s)
HPLC high-pressure, high-performance liquid chromatography
HRMS high-resolution mass spectrometry
conc. concentrated
LC/MS liquid chromatography-coupled mass spectrometry
LiHMDS lithium hexamethyldisilazide
Me methyl
min minute(s)
MS mass spectrometry
NMR nuclear magnetic resonance spectrometry
Pd₂dba₃ tris(dibenzylideneacetone)dipalladium
Ph phenyl
RT room temperature
Rₜ retention time (in HPLC)
THF tetrahydrofuran
UV ultraviolet spectrometry
v/v ratio by volume (of a solution)
XPHOS dicyclohexyl(2′,4′,6′-triisopropylbiphenyl-2-yl)phosphine

### LC/MS Methods:

### Method 1 (LC-MS):

Instrument: Waters ACQUITY SQD UPLC System; column: Waters Acquity UPLC HSS T3 1.8μ 50×1 mm; mobile phase A: 1 l of water+0.25 ml of 99% strength formic acid, mobile phase B: 1 l of acetonitrile+0.25 ml of 99% strength formic acid; gradient: 0.0 min 90% A→1.2 min 5% A→2.0 min 5% A; oven: 50° C.; flow rate: 0.40 ml/min; UV detection: 210-400 nm.

### Method 2 (LC-MS):

Instrument: Micromass Quattro Premier with Waters UPLC Acquity; column: Thermo Hypersil GOLD 1.9μ 50 mm×1 mm; mobile phase A: 1 l of water+0.5 ml of 50% strength formic acid, mobile phase B: 1 l of acetonitrile+0.5 ml of 50% strength formic acid; gradient 0.0 min 97% A→0.5 min 97% A→3.2 min 5% A→4.0 min 5% A; oven: 50° C.; flow rate: 0.3 ml/min; UV detection: 210 nm.

### Method 3 (LC-MS):

Instrument: Waters ACQUITY SQD UPLC System; column: Waters Acquity UPLC HSS T3 1.8μ 30×2 mm; mobile phase A: 1 l of water+0.25 ml of 99% strength formic acid, mobile phase B: 1 l of acetonitrile+0.25 ml of 99% strength formic acid; gradient: 0.0 min 90% A→1.2 min 5% A→2.0 min 5% A; oven: 50° C.; flow rate: 0.60 ml/min; UV detection: 208-400 nm.

### Starting Materials and Intermediates

### Example 1A

### 2-(2-Fluorophenyl)-N-[(6-oxo-1,4,5,6-tetrahydropyridazin-3-yl)methyl]acetamide

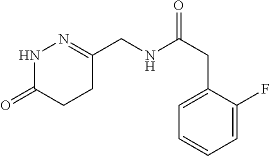 [SMILES: O=C(Cc1ccccc1F)NCC1=NNC(=O)CC1]

200.00 g (1.101 mol) of methyl 5-amino-4-oxopentanoate hydrochloride were initially charged in ethanol (3500 ml), 64.28 ml (1.321 mol) of hydrazine hydrate were added and the mixture was then heated at reflux for 45 min. After cooling, triethylamine (152 ml) was added and the mixture was evaporated to dryness. Water (500 ml) was added to the residue, and the mixture was concentrated. Ethanol (500 ml) was then added, the mixture was concentrated, and then toluene (500 ml) was added twice, followed in each case by evaporation to dryness. The residue (140 g) was dissolved in acetonitrile (500 ml) and, at 0° C., slowly added to a solution of 307.85 g (1.784 mol) of (2-fluorophenyl)acetyl chloride (preparation: Journal of Organic Chemistry; 22; 1957; 879) and 304.86 ml (2.202 mol) of triethylamine in acetonitrile (1500 ml) and molecular sieve. The mixture was stirred at 20° C. for 3 days. The mixture was then filtered and the precipitate was washed with tert-butyl methyl ether and then dried. This gave 458 g of the target compound (90% of theory).

LC-MS (method 1): Rt=0.57 min; MS (EIpos): m/z=264 [M+H]⁺.

### Example 2A

### 2-(2-Fluorophenyl)-N-[(6-oxo-1,6-dihydropyridazin-3-yl)methyl]acetamide

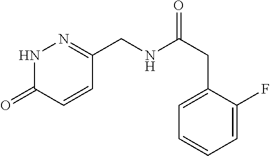 [SMILES: O=C(Cc1ccccc1F)NCc1ccc(=O)[nH]n1]

458 g (1.740 mol) of the compound obtained in Example 1A were initially charged in acetic acid (2250 ml), and the mixture was warmed to 50° C. At this temperature, 98.16 ml (1.914 mol) of bromine were added dropwise with vigorous stirring, and stirring was then continued at 50° C. for 3 h. After cooling, the reaction mixture was concentrated to dryness. The residue was stirred with saturated aqueous sodium bicarbonate solution (4800 ml). The mixture was then filtered and the precipitate was washed with a little water. The filtrate was extracted twice with ethyl acetate. The organic phases were combined, dried and concentrated. This gave 117 g of the target compound (25% of theory).

LC-MS (method 1): Rₜ=0.56 min; MS (EIpos): m/z=262 [M+H]⁺.

¹H NMR (400 MHz, DMSO-d₆): δ [ppm]=3.54 (s, 2H), 4.16 (d, 2H), 6.86 (d, 1H), 7.12-7.16 (m, 2H), 7.27-7.35 (m, 3H), 8.62 (t, 1H), 12.88 (s, 1H).

### Example 3A

### 2-Chloro-7-(2-fluorobenzyl)imidazo[1,5-b]pyridazine

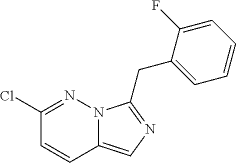 [SMILES: Fc1ccccc1Cc1ncc2ccc(Cl)nn12]

65.00 g (248.79 mmol) of the compound obtained in Example 2A were initially charged in sulfolane (780 ml), 185.52 ml (1.990 mol) of phosphorus oxychloride were added and the mixture was heated to 100° C. for 3 h. Excess phosphorus oxychloride was then distilled off under high vacuum, and the residue was taken up in ethyl acetate and added to a saturated aqueous sodium bicarbonate solution. The mixture was diluted with water and then extracted with ethyl acetate. The organic phases were combined, washed with water, dried over sodium sulfate and concentrated. The residue was purified by chromatography on silica gel (mobile phase: dichloromethane/methanol 20:1→5:1 (v/v)), then washed with water and purified by chromatography on silica gel (mobile phase: dichloromethane/methanol 100:1 v/v). This gave 23.6 g of the target compound (36% of theory).

LC-MS (method 1): Rₜ=1.00 min; MS (EIpos): m/z=262 [M+H]⁺.

¹H NMR (400 MHz, DMSO-d₆): δ [ppm]=4.40 (s, 2H), 6.84 (d, 1H), 7.10-7.33 (m, 4H), 7.55 (s, 1H), 8.19 (d, 1H).

### Example 4A

### 7-(2-Fluorobenzyl)imidazo[1,5-b]pyridazine

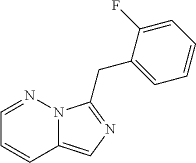 [SMILES: Fc1ccccc1Cc1ncc2cccnn12]

2.004 g of palladium on carbon (5%) were initially charged under argon, and 20.04 g (76.58 mmol) of the compound obtained in Example 3A in ethyl acetate (750 ml) were then added. 21.348 ml (153.159 mmol) of triethylamine were then added, and the reaction mixture was hydrogenated at standard hydrogen pressure and 20° C. for 16 hours. The same amount of catalyst as indicated above was then added, and the reaction mixture was hydrogenated at standard hydrogen pressure and 20° C. for another night. The mixture was then filtered through Celite, the filter cake was washed with ethanol and the filtrate was concentrated and dried under high vacuum. This gave 22.79 g of the target compound (about 100% of theory, contaminated with triethylamine).

LC-MS (method 1): Rt=0.77 min; MS (EIpos): m/z=228 [M+H]⁺.

¹H NMR (400 MHz, DMSO-d₆): δ [ppm]=4.44 (s, 2H), 6.70 (dd, 1H), 7.08-7.31 (m, 4H), 7.45 (s, 1H), 8.09 (dd, 1H), 8.28 (dd, 1H).

### Example 5A

### 5-Bromo-7-(2-fluorobenzyl)imidazo[1,5-b]pyridazine

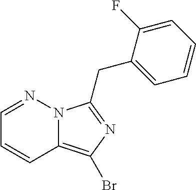 [SMILES: Fc1ccccc1Cc1nc(Br)c2cccnn12]

22.46 g (98.837 mmol) of the compound obtained in Example 4A were initially charged in dichloromethane (400 ml), and 17.591 g (98.837 mmol) of N-bromosuccinimide were added. The mixture was then stirred at 20° C. for 10 min. Water was then added to the reaction mixture, the phases were separated and the organic phase was washed with water. The aqueous phase was extracted twice with dichloromethane and the combined organic phases were washed with saturated aqueous sodium chloride solution, dried over sodium sulfate, filtered and concentrated. This gave 22.78 g of the target compound (75% of theory).

LC-MS (method 1): Rₜ=1.05 min; MS (EIpos): m/z=306, 308 [M+H, bromine pattern]⁺.

¹H NMR (400 MHz, DMSO-d₆): δ [ppm]=4.45 (s, 2H), 6.81 (dd, 1H), 7.12-7.34 (m, 4H), 7.94 (dd, 1H), 8.28 (dd, 1H).

### Example 6A

### 7-(2-Fluorobenzyl)imidazo[1,5-b]pyridazine-5-carbonitrile

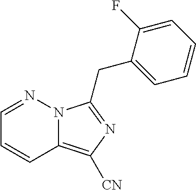 [SMILES: N#Cc1nc(Cc2ccccc2F)n2ncccc12]

1.00 g (3.266 mmol) of the compound obtained in Example 5A were initially charged in dry DMSO (25 ml), 1.170 g (13.066 mmol) of copper(I) cyanide were added and, with stirring, the mixture was heated at 170° C. for 3.5 h. The mixture was filtered through Celite, and the filter cake was washed with ethyl acetate and tetrahydrofuran. The filtrate was then extracted four times with a mixture of saturated aqueous ammonium chloride solution/aqueous ammonia (33%) (3:1, v/v) and washed once with saturated aqueous sodium chloride solution. The phases were separated and the organic phase was dried with sodium sulfate, filtered and concentrated. The residue was treated with ethanol in an ultrasonic bath, and water was then added. The precipiate formed was filtered off, washed with ethanol and then dried under high vacuum. This gave 586 mg of the target compound (71% of theory).

LC-MS (method 1): Rₜ=0.95 min; MS (EIpos): m/z=253 [M+H]⁺.

¹H NMR (400 MHz, DMSO-d₆): δ [ppm]=4.49 (s, 2H), 7.13-7.35 (m, 5H), 8.40 (d, 1H), 8.61 (d, 1H).

### Example 7A

### 7-(2-Fluorobenzyl)imidazo[1,5-b]pyridazine-5-carboximidamide acetate

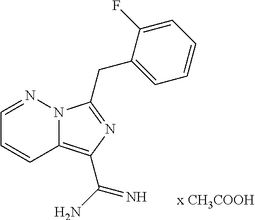 [SMILES: N=C(N)c1nc(Cc2ccccc2F)n2ncccc12]

584 mg (2.315 mmol) of the compound prepared in Example 6A were added to 125 mg (2.315 mmol) of sodium methoxide in methanol (10 ml), and the mixture was stirred at 20° C. for 16 hours. 148 mg (2.778 mmol) of ammonium chloride and acetic acid (0.517 ml) were then added, and the mixture was heated at reflux for 8 h. The reaction mixture was then concentrated to dryness, the residue was taken up in water and ethyl acetate and 1 N aqueous sodium hydroxide solution was added. The phases were separated and the aqueous phase was extracted twice with ethyl acetate. The combined organic phases were dried over sodium sulfate, filtered, concentrated and then dried under high vacuum. This gave 543 mg of the target compound (71% of theory).

LC-MS (method 1): Rt=0.63 min; MS (EIpos): m/z=270 [M+H]⁺.

### Example 8A

### Methyl 3,3-dicyano-2,2-dimethylpropanoate

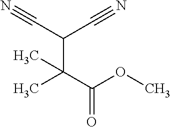

In THF (91 ml), 3 g (45.411 mmol) of malononitrile were added slowly to 1.816 g (45.411 mmol) of sodium hydride (60% in mineral oil). Subsequently, 5.876 ml (45.411 mmol) of methyl 2-bromo-2-methylpropanoate were added and the mixture was stirred at 20° C. for 16 hours. Another 5.876 ml (45.411 mmol) of methyl 2-bromo-2-methylpropanoate were then added, and the mixture was heated at 50° C. for 16 hours. Then yet another 1.762 ml (13.623 mmol) of methyl 2-bromo-2-methylpropanoate were added and the mixture was heated to 50° C. for a further 4 h. Saturated aqueous sodium bicarbonate solution was then added, and the reaction mixture was extracted three times with ethyl acetate. The combined organic phases were washed with saturated aqueous sodium chloride solution, dried over sodium sulfate, filtered and concentrated to dryness. This gave 8.9 g of crude product, which was purified by chromatography on silica gel (cyclohexane-ethyl acetate 4:1).

Yield: 6.47 g (85% of theory)

1H NMR (400 MHz, DMSO-d₆): δ [ppm]=1.40 (s, 6H), 3.74 (s, 3H), 5.27 (s, 1H).

### Example 9A

### 4-Amino-2-[7-(2-fluorobenzyl)imidazo[1,5-b]pyridazin-5-yl]-5,5-dimethyl-5,7-dihydro-6H-pyrrolo[2,3-d]pyrimidin-6-one

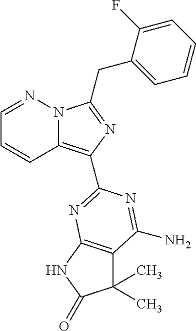 [SMILES: CC1(C)C(=O)Nc2nc(-c3nc(Cc4ccccc4F)n4ncccc34)nc(N)c21]

543 mg (1.649 mmol) of Example 7A were initially charged in tert-butanol (30 ml), and 222 mg (1.979 mmol) of potassium tert-butoxide were added. Subsequently, 301 mg (1.979 mmol) of Example 8A in tert-butanol (20 ml) were added dropwise, and the mixture was heated at reflux for 2 hours. After cooling, water was added and the mixture was extracted three times with ethyl acetate. The combined organic phases were concentrated and a little ethyl acetate and a few drops of ethanol were added to the residue. Diethyl ether was then added. A precipitate was formed. This precipitate was filtered off. Drying under high vacuum gave 290 mg of the target compound (43% of theory).

LC-MS (method 1): Rt=0.85 min; MS (EIpos): m/z=404 [M+H]⁺.

¹H NMR (400 MHz, DMSO-d₆): δ [ppm]=1.32 (s, 6H), 4.49 (s, 2H), 6.66 (s br, 2H), 6.95 (dd, 1H), 7.12-7.22 (m, 2H), 7.24-7.33 (m, 2H), 8.41 (dd, 1H), 8.98 (dd, 1H), 10.86 (s br, 1H).

### Example 10A

### 7-(2-Fluorobenzyl)imidazo[1,5-b]pyridazine-5-carboximidohydrazide

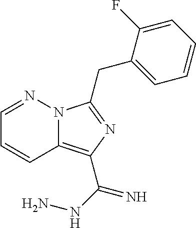 [SMILES: N=C(NN)c1nc(Cc2ccccc2F)n2ncccc12]

1.600 g (4.858 mmol) of Example 7A were dissolved in 40 ml of ethanol, and 1.966 g (19.433 mmol) of triethylamine and 304 mg (4.858 mmol) of hydrazine hydrate (80% strength solution in water) were added at 0° C. The mixture was stirred at RT overnight and then concentrated. The residue was used for the next step without further purification. This gave 1.63 g (73% of theory, purity 62%) of the title compound.

LC-MS (method 2): Rₜ=1.43 min; MS (ESIpos): m/z=285 (M+H)⁺

### Example 11A

### Methyl 2-{3-[7-(2-fluorobenzyl)imidazo[1,5-b]pyridazin-5-yl]-5-hydroxy-1,2,4-triazin-6-yl}-2-methylpropanoate

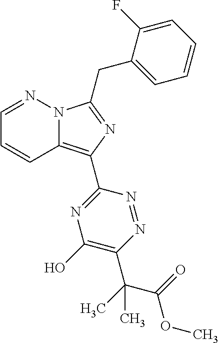 [SMILES: COC(=O)C(C)(C)c1nnc(-c2nc(Cc3ccccc3F)n3ncccc23)nc1O]

1.010 g (5.366 mmol) of dimethyl 2,2-dimethyl-3-oxobutanedioate (described in J. Am. Chem. Soc. 124(14), 3680-3691; 2002) were initially charged in 30 ml of ethanol and heated to reflux. 1.630 g (about 3.577 mmol) of the compound obtained in Example 10A as a suspension in 30 ml of ethanol were slowly added dropwise. The mixture was heated at reflux overnight. After concentration, the residue was used for the next step without further purification. This gave 2.00 g (40% of theory, purity 30%) of the title compound.

LC-MS (method 1): Rₜ=1.01 min; MS (ESIpos): m/z=423 (M+H)⁺

### Example 12A

### 6-Chloro-1-iodo-3-(2,3,6-trifluorobenzyl)imidazo[1,5-a]pyridine

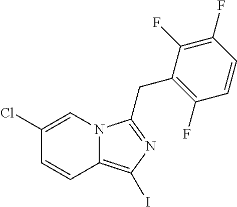 [SMILES: Fc1ccc(F)c(Cc2nc(I)c3ccc(Cl)cn23)c1F]

The title compound was prepared analogously to the procedure in WO 2010/065275, Example 58, pages 46-48.

LC-MS (method 1): Rₜ=1.24 min; MS (ESIpos): m/z=423 (M+H)⁺

### Example 13A

### 6-Chloro-3-(2,3,6-trifluorobenzyl)imidazo[1,5-a]pyridine-1-carbonitrile

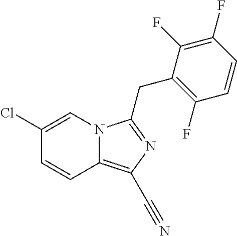 [SMILES: N#Cc1nc(Cc2c(F)ccc(F)c2F)n2cc(Cl)ccc12]

10.00 g (23.664 mmol) of Example 12A and 2.331 g (26.031 mmol) of copper(I) cyanide were stirred in DMSO at 150° C. for 5 h. The mixture was filtered through Celite, the filter cake was washed with ethyl acetate and the filtrate was then extracted four times with a 3:1 mixture of sat. aqueous ammonium chloride solution and conc. aqueous ammonia (33%). The mixture was then washed with saturated aqueous sodium chloride solution, the phases were separated and the organic phase was dried over sodium sulfate, filtered and concentrated. The residue was dried under high vacuum overnight. This gave 7.30 g of the title compound (95% of theory).

LC-MS (method 3): Rt=1.09 min; MS (ESIpos): m/z=322 (M+H)⁺

¹H NMR (400 MHz, DMSO-d₆): δ [ppm]=4.56 (s, 2H), 7.18-7.24 (m, 1H), 7.36 (d, 1H), 7.48-7.57 (m, 1H), 7.87 (d, 1H), 9.00 (s, 1H).

### Example 14A

### 6-Chloro-3-(2,3,6-trifluorobenzyl)imidazo[1,5-a]pyridine-1-carboximidamide

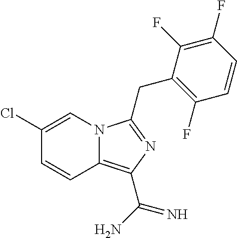 [SMILES: N=C(N)c1nc(Cc2c(F)ccc(F)c2F)n2cc(Cl)ccc12]

3.98 g (12.372 mmol) of Example 13A were reacted analogously to the procedure in WO 2010/065275, Example 58, step H, page 49. This gave 3.54 g of the title compound (84% of theory).

LC-MS (method 1): Rt=0.66 min; MS (ESIpos): m/z=339 (M+H)⁺

¹H NMR (400 MHz, DMSO-d₆): δ [ppm]=4.61 (s, 2H), 7.18-7.24 (m, 1H), 7.47-7.57 (m, 2H), 8.08 (d, 1H), 8.79 (br d, 3H), 9.03 (s, 1H).

### Example 15A

### 4-Amino-2-[6-chloro-3-(2,3,6-trifluorobenzyl)imidazo[1,5-a]pyridin-1-yl]-5,5-dimethyl-5,7-dihydro-6H-pyrrolo[2,3-d]pyrimidin-6-one

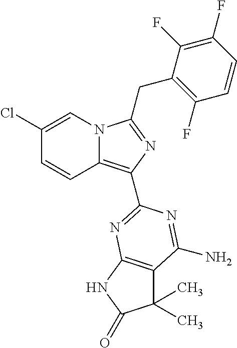 [SMILES: CC1(C)C(=O)Nc2nc(-c3nc(Cc4c(F)ccc(F)c4F)n4cc(Cl)ccc34)nc(N)c21]

The compound is described in WO 2010/065275, Example 59, page 52. This gave 607 mg of the title compound from 500 mg of Example 14A (86% of theory).

LC-MS (method 2): Rₜ=1.95 min; MS (ESIpos): m/z=473 (M+H)⁺

### Example 16A

### 6-Fluoro-1-iodo-3-(3,3,4,4,4-pentafluorobutyl)imidazo[1,5-a]pyridine

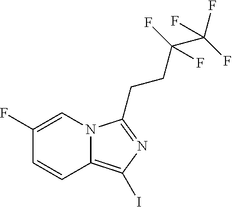 [SMILES: Fc1ccc2c(I)nc(CCC(F)(F)C(F)(F)F)n2c1]

The title compound was prepared in analogy to the procedure in Example 12A.

LC-MS (method 3): Rₜ=1.24 min; MS (ESIpos): m/z=409 (M+H)⁺

### Example 17A

### 6-Fluoro-3-(3,3,4,4,4-pentafluorobutyl)imidazo[1,5-a]pyridine-1-carbonitrile

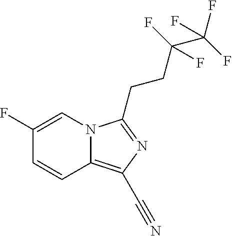 [SMILES: N#Cc1nc(CCC(F)(F)C(F)(F)F)n2cc(F)ccc12]

10.00 g (24.505 mmol) of Example 16A were reacted analogously to the procedure of Example 13A. 6.98 g of the title compound were obtained (92% of theory).

LC-MS (method 3): Rₜ=1.07 min; MS (ESIpos): m/z=308 (M+H)⁺

### Example 18A

### 6-Fluoro-3-(3,3,4,4,4-pentafluorobutyl)imidazo[1,5-a]pyridine-1-carboximidamide

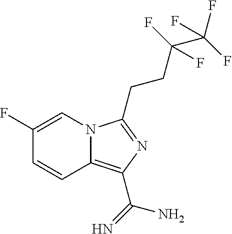 [SMILES: N=C(N)c1nc(CCC(F)(F)C(F)(F)F)n2cc(F)ccc12]

3.810 g (12.402 mmol) of Example 17A were reacted analogously to the procedure of Example 14A. 2.95 g of the title compound were obtained (73% of theory).

LC-MS (method 1): Rt=0.64 min; MS (ESIpos): m/z=325 (M+H)⁺

¹H NMR (400 MHz, DMSO-d₆): δ [ppm]=2.83-2.97 (m, 2H), 2H presumably under the water signal, 7.53-7.58 (m, 1H), 8.16 (dd, 1H), 8.85 (s br, 3H), 8.92 (dd, 1H).

### Example 19A

### 4-Amino-2-[6-fluoro-3-(3,3,4,4,4-pentafluorobutyl)imidazo[1,5-a]pyridin-1-yl]-5,5-dimethyl-5,7-dihydro-6H-pyrrolo[2,3-d]pyrimidin-6-one

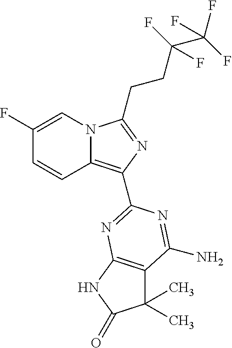 [SMILES: CC1(C)C(=O)Nc2nc(-c3nc(CCC(F)(F)C(F)(F)F)n4cc(F)ccc34)nc(N)c21]

The compound is described in WO 2010/065275, Example 109, page 64. It was prepared analogously to the procedures in Example 15A.

LC-MS (method 2): Rₜ=1.92 min; MS (ESIpos): m/z=459 (M+H)⁺

### Example 20A

### 6-Chloro-3-(2,3,6-trifluorobenzyl)imidazo[1,5-a]pyridine-1-carboximidohydrazide

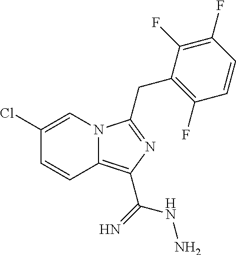 [SMILES: N=C(NN)c1nc(Cc2c(F)ccc(F)c2F)n2cc(Cl)ccc12]

1.700 g (5.019 mmol) of the compound from Example 14A were dissolved in 35 ml of ethanol, and 2.031 g (20.075 mmol) of triethylamine and 0.314 g (5.019 mmol) of hydrazine hydrate (80% strength solution in water) were added at 0° C. The mixture was stirred at RT overnight and then concentrated on a rotary evaporator. This gave a crude compound (2.08 g, 86% of theory, purity 73%) which was directly reacted further.

LC-MS (method 3): Rt=0.65 min; MS (ESIpos): m/z=354 (M+H)±

### Example 21A

### Methyl 2-{3-[6-chloro-3-(2,3,6-trifluorobenzyl)imidazo[1,5-a]pyridin-1-yl]-5-hydroxy-1,2,4-triazin-6-yl}-2-methylpropanoate

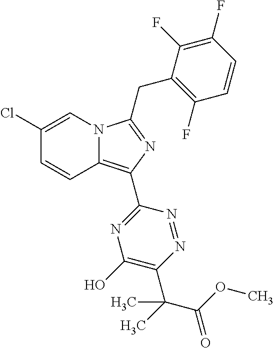 [SMILES: COC(=O)C(C)(C)c1nnc(-c2nc(Cc3c(F)ccc(F)c3F)n3cc(Cl)ccc23)nc1O]

1.222 g (6.492 mmol) of dimethyl 2,2-dimethyl-3-oxobutanedioate (described in J. Am. Chem. Soc. 124(14), 3680-3691; 2002) in 30 ml of ethanol were heated to reflux. The crude substance from Example 20A (about 4.328 mmol) was then dissolved in 20 ml of ethanol and added dropwise. The mixture was heated at reflux overnight. After cooling, a solid was filtered off with suction and washed with ethanol, and the filtrate was concentrated. With stirring, the residue was treated with diethyl ether for 30 min, and a precipiate that formed was filtered off and washed with diethyl ether. This solid was dried under high vacuum overnight. This gave 1.67 g of the title compound (55% of theory, purity 71%).

LC-MS (method 1): Rₜ=1.07 min; MS (ESIpos): m/z=492 (M+H)⁺

### Example 22A

### 6-Fluoro-3-(3,3,4,4,4-pentafluorobutyl)imidazo[1,5-a]pyridine-1-carboximidohydrazide

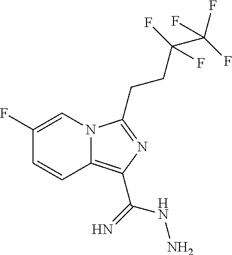 [SMILES: N=C(NN)c1nc(CCC(F)(F)C(F)(F)F)n2cc(F)ccc12]

1.400 g (4.318 mmol) of the compound from Example 18A were dissolved in 30 ml of ethanol, and 1.748 g (17.272 mmol) of triethylamine and 0.270 g (4.318 mmol) of hydrazine hydrate (80% strength solution in water) were added at 0° C. The mixture was stirred at RT overnight and then concentrated on a rotary evaporator. This gave a crude compound (1.70 g, 86% of theory, purity 74%) which was directly reacted further.

LC-MS (method 3): Rₜ=0.65 min; MS (ESIpos): m/z=340 (M+H)⁺

### Example 23A

### Methyl 2-{3-[6-fluoro-3-(3,3,4,4,4-pentafluorobutyl)imidazo[1,5-a]pyridin-1-yl]-5-hydroxy-1,2,4-triazin-6-yl}-2-methylpropanoate

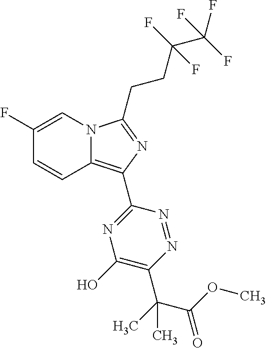 [SMILES: COC(=O)C(C)(C)c1nnc(-c2nc(CCC(F)(F)C(F)(F)F)n3cc(F)ccc23)nc1O]

1.047 g (5.562 mmol) of dimethyl 2,2-dimethyl-3-oxobutanedioate (described in J. Am. Chem. Soc. 124(14), 3680-3691; 2002) in 20 ml of ethanol were heated to reflux. The crude substance from Example 22A (about 3.708 mmol) was then suspended in 20 ml of ethanol and added dropwise. The mixture was heated at reflux overnight and, after cooling, concentrated. With stirring, the residue was treated with diethyl ether for 30 min, and a precipiate that formed was filtered off and washed with diethyl ether. This solid was dried under high vacuum overnight. This gave 1.49 g of the title compound (54% of theory, purity 64%).

LC-MS (method 3): Rt=1.10 min; MS (ESIpos): m/z=478 (M+H)±

### Working Examples

### Example 1

### 2-[7-(2-Fluorobenzyl)imidazo[1,5-b]pyridazin-5-yl]-4-iodo-5,5-dimethyl-5,7-dihydro-6H-pyrrolo[2,3-d]pyrimidin-6-one

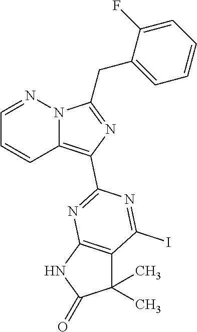 [SMILES: CC1(C)C(=O)Nc2nc(-c3nc(Cc4ccccc4F)n4ncccc34)nc(I)c21]

215 mg (0.535 mmol) of Example 9A were initially charged in isopentyl nitrite (1.548 ml) and diiodomethane (4.045 ml), and the mixture was heated to 85° C. for 3 hours. After cooling, the reaction mixture was concentrated, and the residue was purified by preparative HPLC (acetonitrile:water (+0.05% formic acid) gradient). This gave 79 mg (28% of theory) of the target compound and 20 mg of Example 2.

LC-MS (method 1): Rₜ=1.11 min; MS (EIpos): m/z=515 [M+H]⁺.

¹H NMR (400 MHz, DMSO-d₆): δ [ppm]=1.40 (s, 6H), 4.52 (s, 2H), 6.95 (dd, 1H), 7.12-7.33 (m, 5H), 8.51 (dd, 1H), 8.72 (dd, 1H), 11.64 (s br, 1H).

### Example 2

### 2-[7-(2-Fluorobenzyl)imidazo[1,5-b]pyridazin-5-yl]-4-hydroxy-5,5-dimethyl-5,7-dihydro-6H-pyrrolo[2,3-d]pyrimidin-6-one

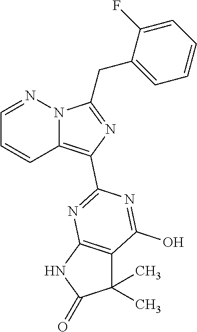 [SMILES: CC1(C)C(=O)Nc2nc(-c3nc(Cc4ccccc4F)n4ncccc34)nc(O)c21]

215 mg (0.535 mmol) of Example 9A were initially charged in isopentyl nitrite (1.548 ml) and diiodomethane (4.045 ml), and the mixture was heated to 85° C. for 3 hours. After cooling, the reaction mixture was concentrated, and the residue was purified by preparative HPLC (acetonitrile:water (+0.05% formic acid) gradient). This gave 20 mg (9% of theory) of the target compound and 79 mg of Example 1.

LC-MS (method 1): Rt=0.90 min; MS (EIpos): m/z=405 [M+H]⁺.

¹H NMR (400 MHz, DMSO-d₆): δ [ppm]=1.31 (s, 6H), 4.55 (s, 2H), 7.12 (t, 1H), 7.18-7.23 (m, 2H), 7.30-7.36 (m, 2H), 8.58 (dd, 1H), 8.63 (d, 1H), 10.95 (s br, 1H), 11.52 (s br, 1H).

### Example 3

### 2-[7-(2-Fluorobenzyl)imidazo[1,5-b]pyridazin-5-yl]-5,5-dimethyl-5,7-dihydro-6H-pyrrolo[2,3-d]pyrimidin-6-one

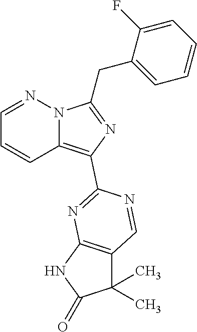 [SMILES: CC1(C)C(=O)Nc2nc(-c3nc(Cc4ccccc4F)n4ncccc34)ncc21]

32 mg of palladium on charcoal (10%) were added to 75 mg (0.146 mmol) of Example 1 in dimethylformamide (10 ml), and the mixture was hydrogenated at standard hydrogen pressure and 20° C. for 4 h. After filtration through Celite, the product was purified by preparative HPLC (acetonitrile:water (+0.05% formic acid) gradient). This gave 30 mg of the target compound (53% of theory).

LC-MS (method 1): Rₜ=0.86 min; MS (EIpos): m/z=389 [M+H]⁺.

¹H NMR (400 MHz, DMSO-d₆): δ [ppm]=1.35 (s, 6H), 4.51 (s, 2H), 7.04 (dd, 1H), 7.12-7.23 (m, 2H), 7.28-7.35 (m, 2H), 8.46 (dd, 1H), 8.51 (s, 1H), 8.88 (dd, 1H), 11.45 (s br, 1H).

### Example 4

### 3-[7-(2-Fluorobenzyl)imidazo[1,5-b]pyridazin-5-yl]-7,7-dimethyl-5,7-dihydro-6H-pyrrolo[2,3-e][1,2,4]triazin-6-one

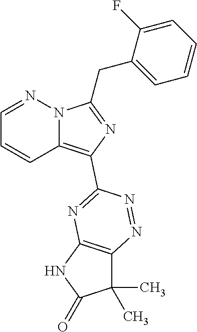 [SMILES: CC1(C)C(=O)Nc2nc(-c3nc(Cc4ccccc4F)n4ncccc34)nnc21]

4.991 ml of phosphoryl chloride were added to 1.00 g (0.733 mmol) of the compound from Example 12A, and the mixture was stirred at RT overnight. The reaction mixture was added to 48 ml of acetonitrile and, with ice cooling, added dropwise to 30.29 ml of concentrated aqueous ammonia solution (33% strength). The mixture was stirred at room temperature for 3 days. The mixture was then concentrated to dryness. The residue was triturated with ethanol, filtered off with suction and washed with ethanol. The filtrate was then concentrated to dryness. The residue was taken up in water and ethyl acetate and the phases were separated. The organic phase was washed four times with water and then once with saturated aqueous sodium chloride solution and subsequently concentrated. The residue was then purified by preparative HPLC (acetonitrile:water (+0.05% formic acid) gradient). This gave 153 mg of the target compound (51% of theory).

LC-MS (method 3): Rₜ=0.90 min; MS (EIpos): m/z=390 [M+H]⁺.

¹H NMR (400 MHz, DMSO-d₆): δ [ppm]=1.43 (s, 6H), 4.54 (s, 2H), 7.09-7.23 (m, 3H), 7.26-7.35 (m, 2H), 8.52 (dd, 1H), 8.78 (dd, 1H), 12.04 (s br, 1H).

### Example 5

### 2-[6-Chloro-3-(2,3,6-trifluorobenzyl)imidazo[1,5-a]pyridin-1-yl]-4-iodo-5,5-dimethyl-5,7-dihydro-6H-pyrrolo[2,3-d]pyrimidin-6-one

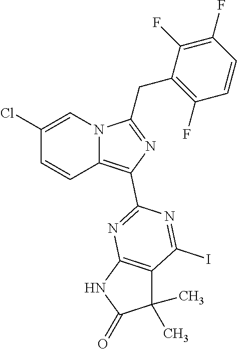 [SMILES: CC1(C)C(=O)Nc2nc(-c3nc(Cc4c(F)ccc(F)c4F)n4cc(Cl)ccc34)nc(I)c21]

556 mg (1.176 mmol) of Example 15A were initially charged in 1,2-dimethoxyethane (14 ml), and 305 mg (1.176 mmol) of cesium iodide, 149 mg (0.588 mmol) of iodine and 67 mg (0.353 mmol) of copper(I) iodide were then added at room temperature. Isopentyl nitrite (0.933 ml) was then added, and the mixture was heated at 60° C. overnight. The next day, another 305 mg (1.176 mmol) of cesium iodide, 149 mg (0.588 mmol) of iodine, 67 mg (0.353 mmol) of copper(I) iodide and isopentyl nitrite (0.933 ml) were added, and the mixture was heated at 60° C. for 3 days. After cooling, the mixture was combined with a smaller batch (starting with 50 mg of Example 15A). The mixture was extracted with ethyl acetate and saturated aqueous sodium thiosulfate, and the phases were separated. The organic phase was extracted two more times with saturated aqueous sodium thiosulfate. The organic phase was then washed with saturated aqueous sodium chloride solution, dried over sodium sulfate, filtered and concentrated, and the residue was purified by preparative HPLC (acetonitrile:water (+0.05% formic acid) gradient). This gave 236 mg of the title compound (31% of theory).

LC-MS (method 1): Rₜ=1.28 min; MS (ESIpos): m/z=584 (M+H)⁺

¹H NMR (400 MHz, DMSO-d₆): δ [ppm]=1.37 (s, 6H), 4.57 (s, 2H), 7.19-7.25 (m, 1H), 7.30 (dd, 1H), 7.48-7.56 (m, 1H), 8.43 (d, 1H), 8.87 (s, 1H), 11.58 (s, 1H).

As well as the title compound, 27 mg (5% of theory, purity 90%) of 2-[6-chloro-3-(2,3,6-trifluorobenzyl)imidazo[1,5-a]pyridin-1-yl]-4-hydroxy-5,5-dimethyl-5,7-dihydro-6H-pyrrolo[2,3-d]pyrimidin-6-one (Example 6) were obtained.

### Example 6

### 2-[6-Chloro-3-(2,3,6-trifluorobenzyl)imidazo[1,5-a]pyridin-1-yl]-4-hydroxy-5,5-dimethyl-5,7-dihydro-6H-pyrrolo[2,3-d]pyrimidin-6-one

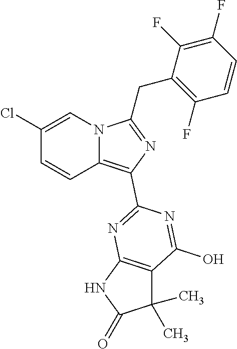 [SMILES: CC1(C)C(=O)Nc2nc(-c3nc(Cc4c(F)ccc(F)c4F)n4cc(Cl)ccc34)nc(O)c21]

Formed as a byproduct in Example 5. Yield: 27 mg (5% of theory, purity 90%).

LC-MS (method 1): Rt=0.97 min; MS (ESIpos): m/z=474 (M+H)⁺

¹H NMR (400 MHz, DMSO-d₆): δ [ppm]=1.28 (s, 6H), 4.59 (s, 2H), 7.18-7.20 (m, 1H), 7.41 (d, 1H), 7.48-7.55 (m, 1H), 8.31 (d, 1H), 8.95 (s, 1H), 10.93 (s, 1H), 11.04 (s, 1H).

### Example 7

### 2-[6-Chloro-3-(2,3,6-trifluorobenzyl)imidazo[1,5-a]pyridin-1-yl]-5,5-dimethyl-5,7-dihydro-6H-pyrrolo[2,3-d]pyrimidin-6-one

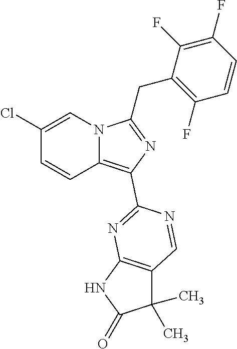 [SMILES: CC1(C)C(=O)Nc2nc(-c3nc(Cc4c(F)ccc(F)c4F)n4cc(Cl)ccc34)ncc21]

100 mg (0.171 mmol) of Example 5 dissolved in DMF (9 ml) were added to 41.1 mg of palladium on carbon (10%) in DMF (1 ml), and the mixture was hydrogenated at standard hydrogen pressure overnight. The mixture was then filtered through Celite, the filter cake was washed with DMF and the filtrate was concentrated to dryness. The residue was purified by preparative HPLC (acetonitrile:water (+0.05% formic acid) gradient). This gave 27 mg of the title compound (34% of theory, purity 96%).

LC-MS (method 2): Rₜ=2.17 min; MS (ESIpos): m/z=458 (M+H)⁺

¹H NMR (400 MHz, DMSO-d₆): δ [ppm]=1.33 (s, 6H), 4.56 (s, 2H), 7.17-7.25 (m, 2H), 7.48-7.56 (m, 1H), 8.46 (s, 1H), 8.51 (dd, 1H), 8.83 (s, 1H), 11.38 (s, 1H).

### Example 8

### 2-[6-Fluoro-3-(3,3,4,4,4-pentafluorobutyl)imidazo[1,5-a]pyridin-1-yl]-4-iodo-5,5-dimethyl-5,7-dihydro-6H-pyrrolo[2,3-d]pyrimidin-6-one

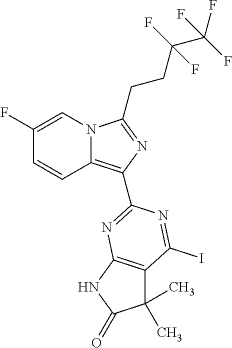 [SMILES: CC1(C)C(=O)Nc2nc(-c3nc(CCC(F)(F)C(F)(F)F)n4cc(F)ccc34)nc(I)c21]

585 mg (1.276 mmol) of Example 19A were reacted analogously to the procedure of Example 5. After purification by preparative HPLC (acetonitrile:water (+0.05% formic acid) gradient), this gave 209 mg of the title compound (28% of theory).

LC-MS (method 1): Rₜ=1.22 min; MS (ESIpos): m/z=570 (M+H)⁺

¹H NMR (400 MHz, DMSO-d₆): δ [ppm]=1.40 (s, 6H), 2.80-2.96 (m, 2H), 2H presumably under the water signal, 7.34 (t, 1H), 8.46 (dd, 1H), 8.73 (d, 1H), 11.62 (s, 1H).

As well as the title compound, 89 mg (15% of theory, purity 87%) of 2-[6-fluoro-3-(3,3,4,4,4-pentafluorobutyl)imidazo[1,5-a]pyridin-1-yl]-4-hydroxy-5,5-dimethyl-5,7-dihydro-6H-pyrrolo[2,3-d]pyrimidin-6-one (Example 9) were obtained.

### Example 9

### 2-[6-Fluoro-3-(3,3,4,4,4-pentafluorobutyl)imidazo[1,5-a]pyridin-1-yl]-4-hydroxy-5,5-dimethyl-5,7-dihydro-6H-pyrrolo[2,3-d]pyrimidin-6-one

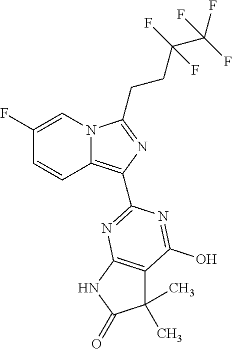 [SMILES: CC1(C)C(=O)Nc2nc(-c3nc(CCC(F)(F)C(F)(F)F)n4cc(F)ccc34)nc(O)c21]

Formed as a byproduct in Example 8. Yield: 89 mg (15% of theory, purity 87%).

LC-MS (method 1): Rt=0.96 min; MS (ESIpos): m/z=460 (M+H)⁺

¹H NMR (400 MHz, DMSO-d₆): δ [ppm]=1.31 (s, 6H), 2.88-3.07 (m, 2H), 2H presumably under the water signal, 7.45 (t, 1H), 8.32 (dd, 1H), 8.81 (d, 1H), 10.91 (s, 1H), 11.56 (s, 1H).

### Example 10

### 2-[6-Fluoro-3-(3,3,4,4,4-pentafluorobutyl)imidazo[1,5-a]pyridin-1-yl]-5,5-dimethyl-5,7-dihydro-6H-pyrrolo[2,3-d]pyrimidin-6-one

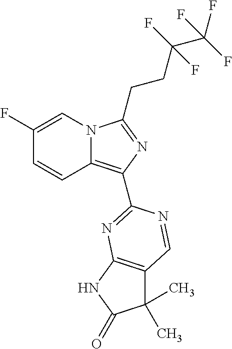 [SMILES: CC1(C)C(=O)Nc2nc(-c3nc(CCC(F)(F)C(F)(F)F)n4cc(F)ccc34)ncc21]

100 mg (0.176 mmol) of Example 8 were hydrogenated analogously to the method in Example 7. This gave 24 mg of the title compound (30% of theory).

LC-MS (method 3): Rt=0.95 min; MS (ESIpos): m/z=444 (M+H)⁺

¹H NMR (400 MHz, DMSO-d₆): δ [ppm]=1.36 (s, 6H), 2.84-2.95 (m, 2H), 2H presumably under the water signal, 7.20-7.25 (m, 1H), 8.50 (s, 1H), 8.55 (dd, 1H), 8.68 (dd, 1H), 11.43 (s, 1H).

### Example 11

### 3-[6-Chloro-3-(2,3,6-trifluorobenzyl)imidazo[1,5-a]pyridin-1-yl]-7,7-dimethyl-5,7-dihydro-6H-pyrrolo[2,3-e][1,2,4]triazin-6-one

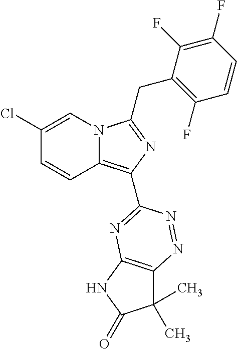 [SMILES: CC1(C)C(=O)Nc2nc(-c3nc(Cc4c(F)ccc(F)c4F)n4cc(Cl)ccc34)nnc21]

16 ml of phosphoryl chloride were added to 1.665 g (about 2.407 mmol, purity 71%) of the compound from Example 21A, and the mixture was stirred at RT overnight. The reaction mixture was dissolved in 160 ml of acetonitrile and, with ice cooling, stirred into 100 ml of concentrated aqueous ammonia solution (33% strength). The mixture was stirred at RT for 3 days. The reaction mixture was then concentrated. The residue was taken up in water and ethanol and stirred at RT for 1 h. The precipitate formed was filtered off with suction and washed with water and a little ethanol. After drying under high vacuum, this gave 899 mg of the title compound (63% of theory, purity 78%). 700 mg of this product were purified by preparative HPLC (acetonitrile:water (+0.05% formic acid) gradient). This gave 212 mg of the title compound (19% of theory).

LC-MS (method 3): Rt=1.03 min; MS (EIpos): m/z=459 [M+H]⁺.

¹H NMR (400 MHz, DMSO-d₆): δ [ppm]=1.40 (s, 6H), 4.58 (s, 2H), 7.20-7.29 (m, 2H), 7.48-7.56 (m, 1H), 8.47 (d, 1H), 8.88 (s, 1H), 11.96 (br s, 1H).

### Example 12

### 3-[6-Fluoro-3-(3,3,4,4,4-pentafluorobutyl)imidazo[1,5-a]pyridin-1-yl]-7,7-dimethyl-5,7-dihydro-6H-pyrrolo[2,3-e][1,2,4]triazin-6-one

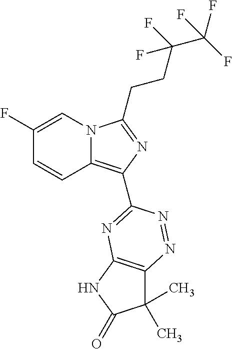 [SMILES: CC1(C)C(=O)Nc2nc(-c3nc(CCC(F)(F)C(F)(F)F)n4cc(F)ccc34)nnc21]

13 ml of phosphoryl chloride were added to 1.490 g (about 2.026 mmol, purity 65%) of the compound from Example 23A, and the mixture was stirred at RT overnight. The reaction mixture was dissolved in 130 ml of acetonitrile and, with ice cooling, stirred into 85 ml of concentrated aqueous ammonia solution (33% strength). The mixture was stirred at RT for 3 days. The reaction mixture was then concentrated. The residue was taken up in water and ethanol and stirred at RT for 1 h. The precipitate formed was filtered off with suction and washed with water and a little ethanol. After drying under high vacuum, this gave 839 mg of the title compound (62% of theory, purity 67%). 200 mg of this product were purified by preparative HPLC (acetonitrile:water (+0.05% formic acid) gradient). This gave 71 mg of the title compound (7% of theory).

LC-MS (method 1): Rt=0.95 min; MS (EIpos): m/z=445 [M+H]⁺.

¹H NMR (400 MHz, DMSO-d₆): δ [ppm]=1.43 (s, 6H), 2.83-2.96 (m, 2H), 2H presumably under the water signal, 7.29-7.34 (m, 1H), 8.51 (dd, 1H), 8.74 (dd, 1H), 12.01 (br s, 1H).

### B. ASSESSMENT OF PHARMACOLOGICAL EFFICACY

The pharmacological effect of the compounds according to the invention can be shown in the following assays:

### B-1. Vasorelaxant Action In Vitro

Rabbits are stunned by a blow to the neck and exsanguinated. The aorta is removed, freed from adhering tissue and divided into rings of a width of 1.5 mm. The rings are placed individually under an initial tension in 5 ml organ baths with Krebs-Henseleit solution which is at 37° C., is gassed with carbogen and has the following composition (in each case mM): sodium chloride 119; potassium chloride: 4.8; calcium chloride dihydrate: 1; magnesium sulfate heptahydrate: 1.4; potassium dihydrogenphosphate: 1.2; sodium bicarbonate: 25; glucose: 10. The contractile force is determined with Statham UC2 cells, amplified and digitalized using A/D transducers (DAS 1802 HC, Keithley Instruments Munich), and recorded in parallel on linear recorders. To obtain a contraction, phenylephrine is added to the bath cumulatively in increasing concentration. After several control cycles, the substance to be investigated is added in each further run in increasing dosage in each case, and the height of the contraction achieved is compared with the height of the contraction reached in the last preceding run. This is used to calculate the concentration needed to reduce the magnitude of the control value by 50% (IC₅₀ value). The standard administration volume is 5 μl; the DMSO content in the bath solution corresponds to 0.1%.

Representative IC₅₀ values for the compounds according to the invention are shown in the table below (Table 1):

TABLE 1

| Example No. | IC₅₀ [nM] |
|---|---|
| 3 | 72 |
| 4 | 42 |
| 11 | 30 |
| 12 | 276 |

### B-2. Effect on a Recombinant Guanylate Cyclase Reporter Cell Line

The cellular activity of the compounds according to the invention is determined using a recombinant guanylate cyclase reporter cell line, as described in F. Wunder et al., Anal. Biochem. 339, 104-112 (2005).

Representative values (MEC=minimum effective concentration) for the compounds according to the invention are shown in the table below (Table 2):

TABLE 2

| Example No. | MEC [μM] |
|---|---|
| 1 | 0.1 |
| 3 | 0.03 |
| 4 | 0.03 |
| 7 | 0.01 |
| 11 | 0.03 |
| 12 | 1.0 |

### B-3. Radiotelemetric Measurement of Blood Pressure on Conscious Spontaneously Hypertensive Rats

A commercially available telemetry system from DATA SCIENCES INTERNATIONAL DSI, USA, is employed for the blood pressure measurements on conscious rats described below.

The system consists of 3 main components:

implantable transmitters (Physiotel® telemetry transmitter)
receivers (Physiotel® receiver) which are linked via a multiplexer (DSI Data Exchange Matrix) to a
data acquisition computer.

The telemetry system makes it possible to continuously record blood pressure, heart rate and body motion of conscious animals in their usual habitat.

### Animal Material

The investigations are carried out on adult female spontaneously hypertensive rats (SHR Okamoto) with a body weight of >200 g. SHR/NCrl from the Okamoto Kyoto School of Medicine, 1963 were a cross of male Wistar Kyoto rats with highly elevated blood pressure and female rats having a slightly elevated blood pressure and at F13 handed over to the U.S. National Institutes of Health.

After transmitter implantation, the experimental animals are housed singly in type 3 Makrolon cages. They have free access to standard feed and water.

The day/night rhythm in the experimental laboratory is changed by the room lighting at 6.00 am and at 7.00 pm.

### Transmitter Implantation

The telemetry transmitters TA11 PA-C40 used are surgically implanted under aseptic conditions in the experimental animals at least 14 days before the first experimental use. The animals instrumented in this way can be employed repeatedly after the wound has healed and the implant has settled.

For the implantation, the fasted animals are anesthetized with pentobarbital (Nembutal, Sanofi: 50 mg/kg i.p.) and shaved and disinfected over a large area of their abdomens. After the abdominal cavity has been opened along the linea alba, the liquid-filled measuring catheter of the system is inserted into the descending aorta in the cranial direction above the bifurcation and fixed with tissue glue (VetBonD™, 3M). The transmitter housing is fixed intraperitoneally to the abdominal wall muscle, and wound is closed layer by layer.

An antibiotic (Tardomyocel COMP, Bayer, 1 ml/kg s.c.) is administered postoperatively for prophylaxis of infection.

### Substances and Solutions

Unless indicated otherwise, the substances to be investigated are administered orally by gavage in each case to a group of animals (n=6). The test substances are dissolved in suitable solvent mixtures, or suspended in 0.5% strength Tylose, appropriate for an administration volume of 5 ml/kg of body weight.

A solvent-treated group of animals is employed as control.

### Test Procedure

The telemetry measuring unit present is configured for 24 animals. Each experiment is recorded under an experiment number (Vyear month day).

Each of the instrumented rats living in the system is assigned a separate receiving antenna (1010 Receiver, DSI).

The implanted transmitters can be activated externally by means of an incorporated magnetic switch and are switched to transmission in the run-up to the experiment. The emitted signals can be detected online by a data acquisition system (Dataquest™ A.R.T. for Windows, DSI) and be appropriately processed. The data are stored in each case in a file created for this purpose and bearing the experiment number.

In the standard procedure, the following are measured for 10-second periods in each case:

systolic blood pressure (SBP)
diastolic blood pressure (DBP)
mean arterial pressure (MAP)
heart rate (HR)
activity (ACT).

The acquisition of measurements is repeated under computer control at 5-minute intervals. The source data obtained as absolute value are corrected in the diagram with the currently measured barometric pressure (Ambient Pressure Reference Monitor; APR-1) and stored as individual data. Further technical details are given in the extensive documentation from the manufacturing company (DSI).

Unless indicated otherwise, the test substances are administered at 9.00 am on the day of the experiment. Following the administration, the parameters described above are measured over 24 hours.

### Evaluation

After the end of the experiment, the acquired individual data are sorted using the analysis software (Dataquest™ A.R.T.™ Analysis). The blank value is assumed to be the time 2 hours before administration, and so the selected data set encompasses the period from 7.00 am on the day of the experiment to 9.00 am the following day.

The data are smoothed over a presettable time by determination of the average (15-minute average) and transferred as a text file to a storage medium. The measured values presorted and compressed in this way are transferred into Excel templates and tabulated. For each day of the experiment, the data obtained are stored in a dedicated file carrying the number of the experiment. Results and test protocols are filed in paper form sorted by numbers.

### Literature

Klaus Witte, Kai Hu, Johanna Swiatek, Claudia Müssig, Georg Ertl and Björn Lemmer: Experimental heart failure in rats: effects on cardiovascular circadian rhythms and on myocardial β-adrenergic signaling. Cardiovasc Res 47 (2): 203-405, 2000; Kozo Okamoto: Spontaneous hypertension in rats. Int Rev Exp Pathol 7: 227-270, 1969; Maarten van den Buuse: Circadian Rhythms of Blood Pressure, Heart Rate, and Locomotor Activity in Spontaneously Hypertensive Rats as Measured With Radio-Telemetry. Physiology & Behavior 55(4): 783-787, 1994

### B-4. Determination of Pharmacokinetic Parameters Following Intravenous and Oral Administration:

The pharmacokinetic parameters of the compounds of the formula (I) according to the invention are determined in male Wistar rats. The administration volume is 5 ml/kg. Intravenous administration is by means of a species-specific plasma/DMSO formulation (99/1). The taking of blood from rats is simplified by inserting a silicone catheter into the right Vena jugularis externa prior to substance administration. The surgical intervention takes place one day prior to the experiment with isofluran anesthesia and administration of an analgetic (atropine/rimadyl (3/1) 0.1 ml s.c.). Substance administration is as an i.v. bolus. Removal of blood from the rats is after 0.033, 0.083, 0.17, 0.5, 1, 2, 3, 4, 6, 8, 24 and 27 hours. The blood is removed into heparinized tubes. The blood plasma is then obtained by centrifugation; if required, it can be stored at −20° C. until further processing.

An internal standard (ZK 228859) is added to the samples of the compounds of the formula (I) according to the invention, calibration samples and QCs, and the protein is precipitated using excess acetonitrile. After addition of an ammonium acetate buffer (0.01 M, pH 6.8) and subsequent vortexing, the mixture is centrifuged at 1000 g and the supernatant is examined by LC-MS/MS (API 4000, AB Sciex). Chromatographic separation is carried out on an Agilent 1100-HPLC. The injection volume is 20 μl. The separation column used is a Phenomenex Gemini 5μ C18 110A 50×3 mm, adjusted to a temperature of 40° C. A binary mobile phase gradient at 500 μl/min is used (A: 0.01M ammonium acetate buffer pH 6.8, B: 0.1% formic acid in acetonitrile): 0 min (90% A), 1 min (90% A), 3 min (10% A), 4 min (10% A), 4.50 min (90% A), 6 min (90% A). The temperature of the Turbo V ion source is 400° C. The following MS instrument parameters are used: curtain gas 15 units, ion spray voltage 4.8 kV, gas 1 50 units, gas 2 40 units, CAD gas 8 units. The substances are quantified by peak heights or areas using extracted ion chromatograms of specific MRM experiments.

The plasma concentration/time plots determined are used to calculate the pharmacokinetic parameters such as AUC, Cₘₐₓ, t_{1/2} (terminal half life), MRT (mean residence time) and CL (clearance), using the validated pharmacokinetic calculation program KinEx (Vers.3).

Since the substance quantification is performed in plasma, it is necessary to determine the blood/plasma distribution of the substance in order to be able to adjust the pharmacokinetic parameters correspondingly. For this purpose, a defined amount of substance is incubated in heparinized whole blood of the species in question in a rocking roller mixer for 20 min. After centrifugation at 1000 g, the plasma concentration is measured (see above) and determined by calculating the quotient of the c_{blood}/cₚₗₐₛₘₐ values.

Following intravenous administration of 0.3 mg/kg of representative compounds according to the invention to rats, the data listed in Table 3 were obtained:

TABLE 3

| Example | 11 | 12 |
|---|---|---|
| CL_{blood} [l/h/kg] | 0.03 | 0.16 |
| Terminal half-life [h] | 7.2 | 5.2 |
| Mean residence time [h] | 9.4 | 4.6 |

### B-5. Metabolic Study

To determine the metabolic profile of the compounds according to the invention, they are incubated with recombinant human cytochrome P450 (CYP) enzymes, liver microsomes or primary fresh hepatocytes from various animal species (e.g. rats, dogs), and also of human origin, in order to obtain and to compare information about substantially the complete hepatic phase I and phase II metabolism, and about the enzymes involved in the metabolism.

The compounds according to the invention were incubated with a concentration of about 0.1-10 μM. To this end, stock solutions of the compounds according to the invention having a concentration of 0.01-1 mM in acetonitrile were prepared, and then pipetted with 1:100 dilution into the incubation mixture. Liver microsomes and recombinant enzymes were incubated at 37° C. in 50 mM potassium phosphate buffer pH 7.4 with and without NADPH-generating system consisting of 1 mM NADP⁺, 10 mM glucose-6-phosphate and 1 unit glucose-6-phosphate dehydrogenase. Primary hepatocytes were incubated in suspension in Williams E medium, likewise at 37° C. After an incubation time of 0-4 h, the incubation reactions were stopped with acetonitrile (final concentration about 30%) and the protein was centrifuged off at about 15 000×g. The samples thus stopped were either analysed directly or stored at −20° C. until analysis.

The analysis is effected by means of high-performance liquid chromatography with ultraviolet and mass spectrometry detection (HPLC-UV-MS/MS). To this end, the supernatants of the incubation samples are chromatographed with suitable C18 reversed-phase columns and variable mobile phase mixtures of acetonitrile and 10 mM aqueous ammonium formate solution or 0.05% formic acid. The UV chromatograms in conjunction with mass spectrometry data serve for identification, structural elucidation and quantitative estimation of the metabolites, and for quantitative metabolic acceptance of the compound according to the invention in the incubation mixtures.

### C. WORKING EXAMPLES OF PHARMACEUTICAL COMPOSITIONS

The compounds according to the invention can be converted to pharmaceutical formulations as follows:

### Tablet:

### Composition:

100 mg of the compound according to the invention, 50 mg of lactose (monohydrate), 50 mg of corn starch (native), 10 mg of polyvinylpyrrolidone (PVP 25) (BASF, Ludwigshafen, Germany) and 2 mg of magnesium stearate.

Tablet weight 212 mg, diameter 8 mm, radius of curvature 12 mm.

### Production:

The mixture of the compound according to the invention, lactose and starch is granulated with a 5% solution (w/w) of the PVP in water. The granules are dried and mixed with the magnesium stearate for 5 minutes. This mixture is pressed with a conventional tableting press (for tablet dimensions see above). The guide value used for the pressing is a pressing force of 15 kN.

Suspension which can be Administered Orally:

### Composition:

1000 mg of the compound according to the invention, 1000 mg of ethanol (96%), 400 mg of Rhodigel® (xanthan gum from FMC, Pennsylvania, USA) and 99 g of water.

A single dose of 100 mg of the compound according to the invention corresponds to 10 ml of oral suspension.

### Production:

The Rhodigel is suspended in ethanol and the compound according to the invention is added to the suspension. The water is added while stirring. The mixture is stirred for about 6 h until swelling of the Rhodigel is complete.

Solution which can be Administered Orally:

### Composition:

500 mg of the compound according to the invention, 2.5 g of polysorbate and 97 g of polyethylene glycol 400. A single dose of 100 mg of the compound according to the invention corresponds to 20 g of oral solution.

### Production:

The compound according to the invention is suspended in the mixture of polyethylene glycol and polysorbate while stirring. The stirring operation is continued until dissolution of the compound according to the invention is complete.

### I.V. Solution:

The compound according to the invention is dissolved in a concentration below the saturation solubility in a physiologically acceptable solvent (e.g. isotonic saline, glucose solution 5% and/or PEG 400 solution 30%). The solution is subjected to sterile filtration and dispensed into sterile and pyrogen-free injection vessels.

1. A compound of the formula (I) 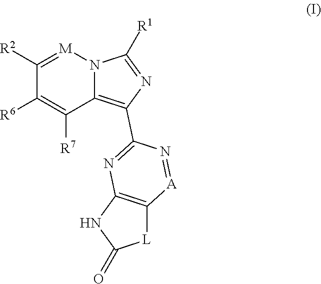
in which
A is nitrogen or CR³,
where
R³ is hydrogen, deuterium, halogen, difluoromethyl, trifluoromethyl, (C₁-C₄)-alkyl, (C₂-C₄)-alkenyl, (C₂-C₄)-alkynyl, cyclopropyl, cyclobutyl, hydroxy, phenyl or 5- or 6-membered heteroaryl,
in which (C₁-C₄)-alkyl, (C₂-C₄)-alkenyl, (C₂-C₄)-alkynyl, phenyl and 5- or 6-membered heteroaryl may be substituted by 1 to 3 substituents independently of one another selected from the group consisting of fluorine, difluoromethyl, trifluoromethyl, (C₁-C₄)-alkyl, difluoromethoxy, trifluoromethoxy, (C₁-C₄)-alkoxy, (C₁-C₄)-alkoxycarbonyl, cyclopropyl and cyclobutyl,
L is a #-CR^{4A}R^{4B}—(CR^{5A}R^{5B})ₚ-## group
where
# is the attachment site to the carbonyl group,
## is the point of attachment to the pyrimidine or triazine ring,
p is a number 0, 1 or 2,
R^{4A} is hydrogen, fluorine, (C₁-C₄)-alkyl, hydroxy or amino,
in which (C₁-C₄)-alkyl may be substituted by 1 to 3 substituents independently of one another selected from the group consisting of fluorine, trifluoromethyl, hydroxy, hydroxycarbonyl, (C₁-C₄)-alkoxycarbonyl and amino,
R^{4B} is hydrogen, fluorine, difluoromethyl, trifluoromethyl, (C₁-C₆)-alkyl, (C₁-C₄)-alkoxycarbonylamino, cyano, (C₃-C₇)-cycloalkyl, difluoromethoxy, trifluoromethoxy, phenyl or a group of the formula -Q-R⁸,
in which (C₁-C₆)-alkyl may be substituted by 1 to 3 substituents independently of one another selected from the group consisting of fluorine, cyano, trifluoromethyl, (C₃-C₇)-cycloalkyl, hydroxy, difluoromethoxy, trifluoromethoxy, (C₁-C₄)-alkoxy, hydroxycarbonyl, (C₁-C₄)-alkoxycarbonyl and amino,
and in which
Q is a bond or (C₁-C₄)-alkanediyl,
R⁸ is —(C═O)ᵣ—OR⁹, —(C═O)ᵣ—NR⁹R¹⁰, —C(═S)—NR⁹R¹⁰, —NR⁹—(C═O)—R¹², —NR⁹—(C═O)—NR¹⁰R¹¹, —NR⁹—SO₂—NR¹⁰R¹¹, —NR⁹—SO₂—R¹², —S(O)ₛ—R¹², —SO₂—NR⁹R¹⁰, 4- to 7-membered heterocyclyl, phenyl or 5- or 6-membered heteroaryl,
in which
r is the number 0 or 1,
s is the number 0, 1 or 2,
R⁹, R¹⁰ and R¹¹ independently of one another are each hydrogen, (C₁-C₆)-alkyl, (C₃-C₈)-cycloalkyl, 4- to 7-membered heterocyclyl, phenyl or 5- or 6-membered heteroaryl,
or
R⁹ and IV° together with the atom(s) to which they are respectively attached form a 4- to 7-membered heterocycle,
in which the 4- to 7-membered heterocycle for its part may be substituted by 1 or 2 substituents independently of one another selected from the group consisting of cyano, trifluoromethyl, (C₁-C₆)-alkyl, hydroxy, oxo, (C₁-C₆)-alkoxy, trifluoromethoxy, (C₁-C₆)-alkoxycarbonyl, amino, mono-(C₁-C₆)-alkyl amino and di-(C₁-C₆)-alkylamino,
or
R¹⁰ and R¹¹ together with the atom(s) to which they are respectively attached form a 4- to 7-membered heterocycle,
in which the 4- to 7-membered heterocycle for its part may be substituted by 1 or 2 substituents independently of one another selected from the group consisting of cyano, trifluoromethyl, (C₁-C₆)-alkyl, hydroxy, oxo, (C₁-C₆)-alkoxy, trifluoromethoxy, (C₁-C₆)-alkoxycarbonyl, amino, mono-(C₁-C₆)-alkyl amino and di-(C₁-C₆)-alkylamino,
R¹² is (C₁-C₆)-alkyl or (C₃-C₇)-cycloalkyl,
or
R⁹ and R¹² together with the atom(s) to which they are respectively attached form a 4- to 7-membered heterocycle,
in which the 4- to 7-membered heterocycle for its part may be substituted by 1 or 2 substituents independently of one another selected from the group consisting of cyano, trifluoromethyl, (C₁-C₆)-alkyl, hydroxy, oxo, (C₁-C₆)-alkoxy, trifluoromethoxy, (C₁-C₆)-alkoxycarbonyl, amino, mono-(C₁-C₆)-alkyl amino and di-(C₁-C₆)-alkylamino,
and
in which 4- to 7-membered heterocyclyl, phenyl and 5- or 6-membered heteroaryl for their part may be substituted by 1 to 3 substituents independently of one another selected from the group consisting of halogen, cyano, difluoromethyl, trifluoromethyl, (C₁-C₆)-alkyl, (C₃-C₇)-cycloalkyl, hydroxy, oxo, thioxo and (C₁-C₄)-alkoxy,
and
in which the aforementioned (C₁-C₄)-alkyl, (C₁-C₆)-alkyl, (C₃-C₈)-cycloalkyl and 4- to 7-membered heterocyclyl groups, unless stated otherwise, may each independently of one another additionally be substituted by 1 to 3 substituents independently of one another selected from the group consisting of fluorine, difluoromethyl, trifluoromethyl, (C₁-C₆)-alkyl, (C₃-C₇)-cycloalkyl, hydroxy, difluoromethoxy, trifluoromethoxy, (C₁-C₄)-alkoxy, hydroxycarbonyl, (C₁-C₄)-alkoxycarbonyl, amino, phenyl, 4- to 7-membered heterocyclyl and 5- or 6-membered heteroaryl,
or
R^{4A} and R^{4B} together with the carbon atom to which they are bonded form a (C₂-C₄)-alkenyl group, an oxo group, a 3- to 6-membered carbocycle or a 4- to 7-membered heterocycle,
in which the 3- to 6-membered carbocycle and the 4- to 7-membered heterocycle may be substituted by 1 or 2 substituents selected independently from the group of fluorine and (C₁-C₄)-alkyl,
R^{5A} is hydrogen, fluorine, (C₁-C₄)-alkyl, (C₁-C₄)-alkoxycarbonyl or hydroxy,
R^{5B} is hydrogen, fluorine, (C₁-C₄)-alkyl or trifluoromethyl,
M is CH or N,
R¹ is (C₁-C₆)-alkyl, (C₃-C₈)-cycloalkylmethyl, benzyl or 5- or 6-membered heteroarylmethyl,
where (C₁-C₆)-alkyl is substituted by a substituent selected from the group consisting of difluoromethyl and trifluoromethyl,
where (C₁-C₆)-alkyl may be substituted by 1 to 3 fluorine substituents,
where (C₃-C₈)-cycloalkylmethyl may be substituted by 1 or 2 substituents independently of one another selected from the group consisting of fluorine, methyl and methoxy,
where benzyl is substituted by 1 to 3 fluorine substituents,
and
where 5- and 6-membered heteroarylmethyl may be substituted by 1 or 2 fluorine substituents,
R² is hydrogen, cyano, halogen, difluoromethyl, trifluoromethyl, (C₁-C₄)-alkyl or (C₃-C₇)-cycloalkyl,
R⁶ is hydrogen, cyano, halogen, difluoromethyl, trifluoromethyl, (C₁-C₄)-alkyl or (C₃-C₇)-cycloalkyl,
R⁷ is hydrogen, cyano, difluoromethyl, trifluoromethyl, (C₁-C₄)-alkyl or (C₃-C₇)-cycloalkyl,
or a salt thereof.

2. The compound of claim 1 wherein
A is nitrogen or CR³,
where
R³ is hydrogen, deuterium, fluorine, iodine, difluoromethyl, trifluoromethyl, (C₁-C₄)-alkyl, vinyl, allyl, ethynyl, cyclopropyl, cyclobutyl, hydroxy, pyrazolyl or pyridyl,
where (C₁-C₄)-alkyl, vinyl, allyl, ethynyl and pyridyl may be substituted by 1 or 2 substituents independently of one another selected from the group consisting of methyl, cyclopropyl and cyclobutyl,
L is a #-CR^{4A}R^{4B}—(CR^{5A}R^{5B})ₚ-## group
where
# is the attachment site to the carbonyl group,
## is the point of attachment to the pyrimidine or triazine ring,
p is a number 0 or 1,
R^{4A} is hydrogen, fluorine, methyl, ethyl, hydroxy or amino,
R^{4B} is hydrogen, fluorine, difluoromethyl, trifluoromethyl, (C₁-C₄)-alkyl, methoxycarbonylamino, cyano, cyclopropyl, cyclobutyl, cyclopentyl, phenyl or a group of the formula -Q-R⁸,
in which (C₁-C₄)-alkyl may be substituted by 1 to 3 substituents independently of one another selected from the group consisting of fluorine, cyano, trifluoromethyl, cyclopropyl, cyclobutyl, cyclopentyl, hydroxy, difluoromethoxy, trifluoromethoxy, methoxy, ethoxy, hydroxycarbonyl, methoxycarbonyl, ethoxycarbonyl and amino,
and in which
Q is a bond or methylene,
R⁸ is —(C═O)ᵣ—NR⁹R¹⁰, —C(═S)—NR⁹R¹⁰, oxadiazolonyl, oxadiazolthionyl, phenyl, oxazolyl, thiazolyl, pyrazolyl, triazolyl, oxadiazolyl, thiadiazolyl, pyridyl, pyrimidinyl or pyrazinyl,
in which
r is the number 0 or 1,
R⁹ and R¹⁰ are each independently of one another hydrogen, methyl, ethyl, isopropyl, cyclopropyl, cyclobutyl, cyclopentyl, oxetanyl, azetidinyl, tetrahydrofuranyl, pyrrolidinyl, tetrahydropyranyl, piperidinyl, piperazinyl, morpholinyl, phenyl, pyrazolyl or pyridyl,
in which methyl, ethyl and isopropyl may additionally be substituted by 1 or 2 substituents independently of one another selected from the group consisting of fluorine, difluoromethyl, trifluoromethyl, cyclopropyl, cyclobutyl, cyclopentyl, hydroxy, difluoromethoxy, trifluoromethoxy, methoxy, ethoxy, hydroxycarbonyl, methoxycarbonyl, ethoxycarbonyl and amino,
and
in which oxadiazolonyl, oxadiazolthionyl, phenyl, oxazolyl, thiazolyl, pyrazolyl, triazolyl, oxadiazolyl, thiadiazolyl, pyridyl, pyrimidinyl and pyrazinyl for their part may be substituted by 1 or 2 substituents independently of one another selected from the group consisting of fluorine, chlorine, cyano, difluoromethyl, trifluoromethyl, methyl, ethyl, isopropyl, 2,2,2-trifluoroethyl, 1,1,2,2,2-pentafluoroethyl, cyclopropyl, cyclobutyl, cyclopropylmethyl, cyclobutylmethyl, hydroxy, methoxy and ethoxy,
or
R^{4A} and R^{4B} together with the carbon atom to which they are attached form a cyclopropyl, cyclobutyl, cyclopentyl, azetidinyl, tetrahydrofuranyl, pyrrolidinyl or tetrahydropyranyl ring,
in which the cyclopropyl, cyclobutyl, cyclopentyl, azetidinyl, tetrahydrofuranyl, pyrrolidinyl and tetrahydropyranyl ring may be substituted by 1 or 2 substituents independently of one another selected from the group consisting of fluorine and methyl,
R^{5A} is hydrogen, fluorine, methyl, ethyl or hydroxy,
R^{5B} is hydrogen, fluorine, methyl, ethyl or trifluoromethyl,
M is CH or N,
R¹ is 3,3,3-trifluoroprop-1-yl, 2,2,3,3,3-pentafluoroprop-1-yl, 4,4,4-trifluorobut-1-yl, 3,3,4,4-tetrafluorobut-1-yl, 3,3,4,4,4-pentafluorobut-1-yl, (C₃-C₆)-cycloalkylmethyl, benzyl, thienylmethyl, pyridylmethyl, pyrimidinylmethyl, pyrazinylmethyl or pyridazinylmethyl,
where benzyl is substituted by 1 to 3 fluorine substituents,
and
where (C₃-C₆)-cycloalkylmethyl, thienylmethyl, pyridylmethyl, pyrimidinylmethyl, pyrazinylmethyl and pyridazinylmethyl may be substituted by 1 or 2 fluorine substituents,
R² is hydrogen, fluorine or chlorine,
R⁶ is hydrogen, fluorine, chlorine or methyl,
R⁷ represents hydrogen or methyl,
or a salt thereof.

3. The compound of claim 1 wherein
A is nitrogen or CR³,
where
R³ represents hydrogen,
L is a #-CR^{4A}R^{4B}—(CR^{5A}R^{5B})ₚ-## group
where
# is the attachment site to the carbonyl group,
## is the point of attachment to the pyrimidine or triazine ring,
p is a number 0,
R^{4A} is hydrogen, fluorine, methyl, ethyl, hydroxy or amino,
R^{4B} is hydrogen, fluorine, difluoromethyl, trifluoromethyl, methyl, ethyl, methoxycarbonylamino, cyclopropyl, cyclobutyl, cyclopentyl or a group of the formula -Q-R⁸,
in which methyl and ethyl may be substituted by 1 to 3 substituents independently of one another selected from the group consisting of fluorine, cyano, trifluoromethyl, cyclopropyl, cyclobutyl, hydroxy, difluoromethoxy, trifluoromethoxy, methoxy, ethoxy, hydroxycarbonyl, methoxycarbonyl, ethoxycarbonyl and amino,
and in which
Q is a bond,
R⁸ is —(C═O)ᵣ—NR⁹R¹⁰, phenyl, thiazolyl, triazolyl, oxadiazolyl, thiadiazolyl or pyrimidinyl,
in which
r is the number 1,
R⁹ and R¹⁰ independently of one another are each hydrogen or cyclopropyl,
and
in which phenyl, thiazolyl, triazolyl, oxadiazolyl, thiadiazolyl and pyrimidinyl for their part may be substituted 1 or 2 substituents independently of one another selected from the group consisting of fluorine, difluoromethyl, trifluoromethyl, methyl, ethyl, isopropyl, 2,2,2-trifluoroethyl, 1,1,2,2,2-pentafluoroethyl, cyclopropyl, cyclobutyl, cyclopropylmethyl and cyclobutylmethyl,
or
R^{4A} and R^{4B} together with the carbon atom to which they are attached form a cyclopropyl, cyclobutyl, cyclopentyl, azetidinyl, tetrahydrofuranyl, pyrrolidinyl or tetrahydropyranyl ring,
in which the cyclopropyl, cyclobutyl, cyclopentyl, azetidinyl, tetrahydrofuranyl, pyrrolidinyl and tetrahydropyranyl ring may be substituted by 1 or 2 substituents independently of one another selected from the group consisting of fluorine and methyl,
M is CH or N,
R¹ is 4,4,4-trifluorobut-1-yl, 3,3,4,4-tetrafluorobut-1-yl, 3,3,4,4,4-pentafluorobut-1-yl or benzyl,
where benzyl is substituted by 1 to 3 fluorine substituents,
R² is fluorine or chlorine if M is CH,
or
R² is hydrogen if M is N,
R⁶ is hydrogen,
R⁷ represents hydrogen,
or a salt thereof.

4. The compound of claim 1, wherein
A is nitrogen or CR³,
where
R³ represents hydrogen,
L is a #-CR^{4A}R^{4B}—(CR^{5A}R^{5B})ₚ-## group
where
# is the attachment site to the carbonyl group,
## is the point of attachment to the pyrimidine or triazine ring,
p is a number 0,
R^{4A} is hydrogen, fluorine, methyl or hydroxy,
R^{4B} is hydrogen, fluorine, trifluoromethyl, 2,2,2-trifluoroethyl or methyl,
M is CH or N,
R¹ is 4,4,4-trifluorobut-1-yl, 3,3,4,4-tetrafluorobut-1-yl, 3,3,4,4,4-pentafluorobut-1-yl or benzyl,
where benzyl is substituted by 1 to 3 fluorine substituents,
R² is fluorine or chlorine if M is CH,
or
R² is hydrogen if M is N,
R⁶ is hydrogen,
R⁷ represents hydrogen,
or a salt thereof.

5. A process for preparing a compound of formula (I) as defined in claim 1 to 4, comprising
[A] cyclizing a compound of formula (II) 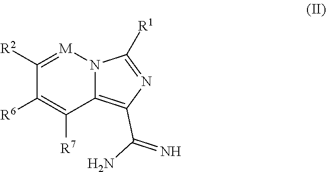
in which M, R¹, R², R⁶ and R⁷ each have the meaning given in claim 1,
in an inert solvent in the presence of a suitable base with a compound of the formula (III) 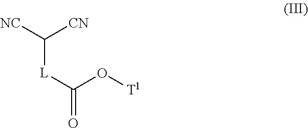
in which L has the meaning given in claim 1 and
T¹ is (C₁-C₄)-alkyl
to give a compound of formula (IV) 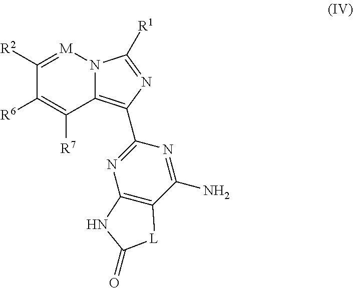
in which M, L, R¹, R², R⁶ and R⁷ each have the meanings given in claim 1,
converting the compound of formula (IV) in an inert solvent using isopentyl nitrite and a halogen equivalent into a compound of formula (I-A) 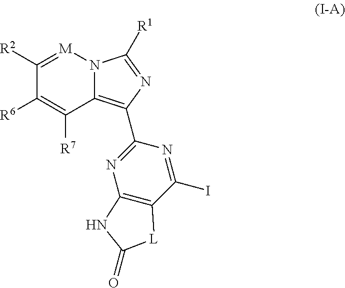
in which M, L, R¹, R², R⁶ and R⁷ each have the meanings given in claim 1,
or
[B] reacting the compound of formula (I-A) is reacted in an inert solvent in the presence of a suitable transition metal catalyst to give a compound of formula (I-B) 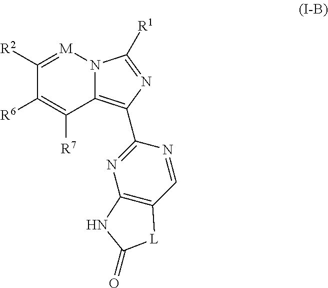
in which M, L, R¹, R², R⁶ and R⁷ each have the meanings given in claim 1,
or
[C] reacting the compound of formula (II) in an inert solvent in the presence of a suitable base with a compound of formula (V) 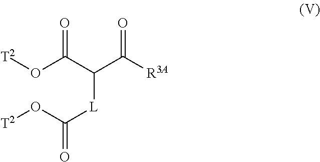
in which L has the meaning given in claim 1,
R^{3A} is hydrogen, halogen, (C₁-C₄)-alkyl or hydroxy and
T² is (C₁-C₄)-alkyl,
to give a compound of formula (VI) 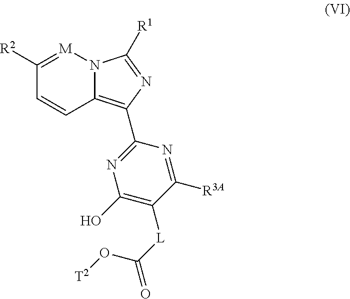
in which M, L, R¹, R², R^{3A}, R⁶, R⁷ and T² each have the meanings given above,
converting the compound of formula (VI) with phosphoryl chloride into a compound of the formula (VII) 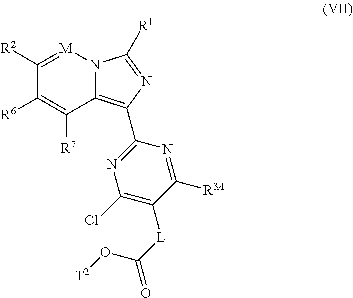
in which M, L, R¹, R², R^{3A}, R⁶, R⁷ and T² each have the meanings given above,
converting the compound of formula (VII) in an inert solvent into a corresponding azide compound, which is reduced directly to give a compound of formula (VIII) 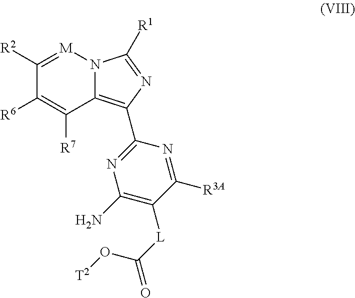
in which M, L, R¹, R², R^{3A}, R⁶, R⁷ and T² each have the meanings given above,
and reacting the compound of formula (VIII) in an inert solvent, in the presence of a suitable base to give a compound of the formula (I-C) 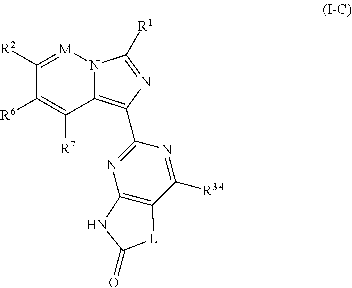
in which M, L, R¹, R² and R^{3A}, R⁶, R⁷ each have the meanings given above,
or
[D] reacting the compound of formula (II) in an inert solvent in the presence of a suitable base with hydrazine hydrate to give a compound of formula (IX) 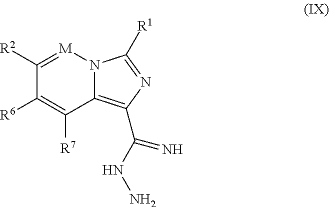
in which M, R¹, R², R⁶ and R⁷ each have the meanings given in claim 1,
reacting the compound of formula (IX) in an inert solvent with a compound of the formula (X) 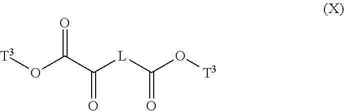
in which L has the meaning given in claim 1 and
T³ is (C₁-C₄)-alkyl
to give a compound of the formula (XI) 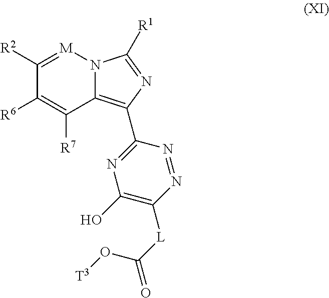
in which M, L, R¹, R², R⁶, R⁷ and T³ each have the meanings given in claim 1,
converting the compound of formula (XI) into a compound of formula (XII) 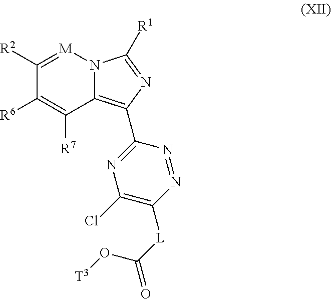
in which M, L, R¹, R², R⁶, R⁷ and T³ each have the meanings given in claim 1,
and reacting the compound of formula (XII) directly with ammonia to give a compound of formula (XIII) 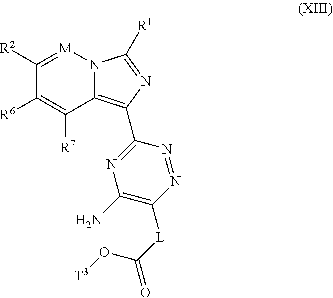
in which M, L, R¹, R², R⁶, R⁷ and T³ each have the meanings given in claim 1,
and cyclizing the compound of formula (XIII) in an inert solvent, optionally in the presence of a suitable base, to give a compound of the formula (I-D) 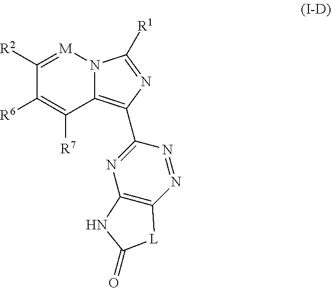
in which M, L, R¹, R², R⁶ and R⁷ each have the meanings given in claim 1,
and the resulting compound of formulae (I-A), (I-B), (I-C) or (I-D) is optionally converted with the appropriate (i) solvent and/or (ii) acid or base into a solvates, salt and/or solvate of a salt thereof.

6. (canceled)

7. (canceled)

8. (canceled)

9. A pharmaceutical composition comprising a compound of claim 1 and an inert, nontoxic, pharmaceutically suitable excipient.

10. The pharmaceutical composition of claim 9, further comprising an active compound selected from the group consisting of an organic nitrate, an NO donor, a cGMP-PDE inhibitor, an antithrombotic agent, a hypotensive agent and a lipid metabolism modifier.

11. (canceled)

12. A method for the treatment and/or prophylaxis of heart failure, angina pectoris, hypertension, pulmonary hypertension, ischemias, vascular disorders, kidney failure, thromboembolic disorders, fibrotic disorders and arteriosclerosis comprising administering to a human or animal in need thereof an effective amount of at least one compound of claim 1.

13. A method for the treatment and/or prophylaxis of heart failure, angina pectoris, hypertension, pulmonary hypertension, ischemias, vascular disorders, kidney failure, thromboembolic disorders, fibrotic disorders and arteriosclerosis, comprising administering to a human or animal in need thereof an effective amount of the pharmaceutical composition of claim 9.
